SEQUENCE LISTING (28 sequences)

SEQ ID NO: 1 - DNA, 1879 bp, Homo sapiens sialophorin
gcctcgggaggtggtggagtgacctggccccagtgctgcgtccttatcagccgagccggtcccagctcttgctcctgcctgtttgcctggaaatggccacgcttctccttctccttggggtgctggtggtaagcccagacgctctggggagcacaacagcagtgcagacacccacctccggagagcctttggtctctactagcgagcccctgagctcaaagatgtacaccacttcaataacaagtgaccctaaggccgacagcactggggaccagacctcagccctacctccctcaacttccatcaatgagggatcccctctttggacttccattggtgccagcactggttcccctttacctgagccaacaacctaccaggaagtttccatcaagatgtcatcagtgccccaggaaacccctcatgcaaccagtcatcctgctgttcccataacagcaaactctctaggatcccacaccgtgacaggtggaaccataacaacgaactctccagaaacctccagtaggaccagtggagcccctgttaccacggcagctagctctctggagacctccagaggcacctctggaccccctcttaccatggcaactgtctctctggagacttccaaaggcacctctggaccccctgttaccatggcaactgactctctggagacctccactgggaccactggaccccctgttaccatgacaactggctctctggagccctccagcggggccagtggaccccaggtctctagcgtaaaactatctacaatgatgtctccaacgacctccaccaacgcaagcactgtgcccttccggaacccagatgagaactcacgaggcatgctgccagtggctgtgcttgtggccctgctggcggtcatagtcctcgtggctctgctcctgctgtggcgccggcggcagaagcggcggactggggccctcgtgctgagcagaggtggcaagcgtaacggggtggtggacgcctgggctgggccagcccaggtccctgaggagggggccgtgacagtgaccgtgggagggtccgggggcgacaagggctctgggttccccgatggggaggggtctagccgtcggcccacgctcaccactttctttggcagacggaagtctcgccagggctccctggcgatggaggagctgaagtctgggtcaggccccagcctcaaaggggaggaggagccactggtggccagtgaggatggggctgtggacgccccagctcctgatgagcccgaagggggagacggggctgccccttaagtgtcggtgaatagtgaggctggaggccggaatctcagccagcctccagcaccttccctctcaccatcccactgccccctcgctcccatgtttccacccggcaccctgatcctcacccgaatctcctttttttttttcttttgagacagagtttcgctttgtcgcccaggctggagtgcaatgcacgatctcagttcactgcaacctctgcctcctaagttcaggcgattctcctgcctcagcttcccgagtaactgagattacaggcacccaccaccatgcccagctgcttttttgtatttttggtagagatggggtttcaccatgttggctaggctggtctcaaactcctgacctcaggtgatctacctgcctcagcctcccaaagtgctgagattacagacatgagcctccgcgccttgcctcctcacccacctcttcactctgaatcctcatgaggcttctcagccctggatttcctgctgccatcctcacccagcacccacaactagcgcctgggcagggcagggctggcacctctcaacgtctgtggactgaatgaataaaccctcctcatccacccc

SEQ ID NO: 2 - protein, 400 aa, Homo sapiens sialophorin
MATLLLLLGVLVVSPDALGSTTAVQTPTSGEPLVSTSEPLSSKMYTTSITSDPKADSTGDQTSALPPSTSINEGSPLWTSIGASTGSPLPEPTTYQEVSIKMSSVPQETPHATSHPAVPITANSLGSHTVTGGTITTNSPETSSRTSGAPVTTAASSLETSRGTSGPPLTMATVSLETSKGTSGPPVTMATDSLETSTGTTGPPVTMTTGSLEPSSGASGPQVSSVKLSTMMSPTTSTNASTVPFRNPDENSRGMLPVAVLVALLAVIVLVALLLLWRRRQKRRTGALVLSRGGKRNGVVDAWAGPAQVPEEGAVTVTVGGSGGDKGSGFPDGEGSSRRPTLTTFFGRRKSRQGSLAMEELKSGSGPSLKGEEEPLVASEDGAVDAPAPDEPEGGDGAAP

SEQ ID NO: 3 - DNA, 1893 bp, Homo sapiens
cttccctgcctccctcaggtcccagctcttgctcctgcctgtttgcctggaaatggccacgcttctccttctccttggggtgctggtggtaagcccagacgctctggggagcacaacagcagtgcagacacccacctccggagagcctttggtctctactagcgagcccctgagctcaaagatgtacaccacttcaataacaagtgaccctaaggccgacagcactggggaccagacctcagccctacctccctcaacttccatcaatgagggatcccctctttggacttccattggtgccagcactggttcccctttacctgagccaacaacctaccaggaagtttccatcaagatgtcatcagtgccccaggaaacccctcatgcaaccagtcatcctgctgttcccataacagcaaactctctaggatcccacaccgtgacaggtggaaccataacaacgaactctccagaaacctccagtaggaccagtggagcccctgttaccacggcagctagctctctggagacctccagaggcacctctggaccccctcttaccatggcaactgtctctctggagacttccaaaggcacctctggaccccctgttaccatggcaactgactctctggagacctccactgggaccactggaccccctgttaccatgacaactggctctctggagccctccagcggggccagtggaccccaggtctctagcgtaaaactatctacaatgatgtctccaacgacctccaccaacgcaagcactgtgcccttccggaacccagatgagaactcacgaggcatgctgccagtggctgtgcttgtggccctgctggcggtcatagtcctcgtggctctgctcctgctgtggcgccggcggcagaagcggcggactggggccctcgtgctgagcagaggcggcaagcgtaacggggtggtggacgcctgggctgggccagcccaggtccctgaggagggggccgtgacagtgaccgtgggagggtccgggggcgacaagggctctgggttccccgatggggaggggtctagccgtcggcccacgctcaccactttctttggcagacggaagtctcgccagggctccctggcgatggaggagctgaagtctgggtcaggccccagcctcaaaggggaggaggagccactggtggccagtgaggatggggctgtggacgccccagctcctgatgagcccgaagggggagacggggctgccccttaagtgtcggtgaatagtgaggctggaggccggaatctcagccagcctccagcaccttccctctcaccatcccactgccccctcgctcccatgtttccacccggcaccctgatcctcacccgaatctcctttttttttttcttttgagacagagtttcgctttgtcgcccaggctggagtgcaatgcacgatctcagttcactgcaacctctgcctcctaagttcaggcgattctcctgcctcagcttcccgagtaactgagattacaggcacccaccaccatgcccagctgcttttttgtatttttggtagagatggggtttcaccatgttggctaggctggtctcaaactcctgacctcaggtgatctacctgcctcagcctcccaaagtgctgagattacagacatgagcctccgcgccttgcctcctcacccacctcttcactctgaatcctcatgaggcttctcagccctggatttcctgctgccatcctcacccagcacccacaactagcgcctgggcagggcagggctggcacctctcaacgtctgtggactgaatgaataaaccctcctcatccacccctaaaaaaaaaaaaaaaaaaaaaaaaaaaaaaaaaaaaaaaaaaaaaaaaaaaaa

SEQ ID NO: 4 - protein, 400 aa, Homo sapiens; identical to SEQ ID NO: 2

SEQ ID NO: 5 - DNA, 1924 bp, Human leukosialin
cctctgagcccagccctccctagcatcaccacttccatcccattcctcagccaagagccaggaatcctgattccagatcccacgcttccctgcctccctcaggtcccagctcttgctcctgcctgtttgcctggaaatggccacgcttctccttctccttggggtgctggtggtaagcccagacgctctggggagcacaacagcagtgcagacacccacctccggagagcctttggtctctactagcgagcccctgagctcaaagatgtacaccacttcaataacaagtgaccctaaggccgacagcactggggaccagacctcagccctacctccctcaacttccatcaatgagggatcccctctttggacttccattggtgccagcactggttcccctttacctgagccaacaacctaccaggaagtttccatcaagatgtcatcagtgccccaggaaacccctcatgcaaccagtcatcctgctgttcccataacagcaaactctctaggatcccacaccgtgacaggtggaaccataacaacgaactctccagaaacctccagtaggaccagtggagcccctgttaccacggcagctagctctctggagacctccagaggcacctctggaccccctcttaccatggcaactgtctctctggagacttccaaaggcacctctggaccccctgttaccatggcaactgactctctggagacctccactgggaccactggaccccctgttaccatgacaactggctctctggagccctccagcggggccagtggaccccaggtctctagcgtaaaactatctacaatgatgtctccaacgacctccaccaacgcaagcactgtgcccttccggaacccagatgagaactcacgaggcatgctgccagtggctgtgcttgtggccctgctggcggtcatagtcctcgtggctctgctcctgctgtggcgccggcggcagaagcggcggactggggccctcgtgctgagcagaggcggcaagcgtaacggggtggtggacgcctgggctgggccagcccaggtccctgaggagggggccgtgacagtgaccgtgggagggtccgggggcgacaagggctctgggttccccgatggggaggggtctagccgtcggcccacgctcaccactttctttggcagacggaagtctcgccagggctccctggcgatggaggagctgaagtctgggtcaggccccagcctcaaaggggaggaggagccactggtggccagtgaggatggggctgtggacgccccagctcctgatgagcccgaagggggagacggggctgccccttaagtgtcggtgaatagtgaggctggaggccgcaatctcagccagcctccagcaccttccctctcaccatcccactgccccctcgctcccatgtttccacccggcaccctgatcctcacccgaatctcctttttttttttcttttgagacagagtttcgctttgtcgcccaggctggagtgcaatgcacgatctcagttcactgcaacctctgcctcctaagttcaggcgattctcctgcctcagcttcccgagtaactgagattacaggcacccaccaccatgcccagctgcttttttgtatttttggtagagatggggtttcaccatgttggctaggctggtctcaaactcctgacctcaggtgatctacctgcctcagcctcccaaagtgctgagattacagacatgagcctccgcgccttgcctcctcacccacctcttcactctgaatcctcatgaggcttctcagccctggatttcctgctgccatcctcacccagcacccacaactagcgcctgggcagggcagggctggcacctctcaacgtctgtggactgaatgaataaaccctcctcatccacccct

SEQ ID NO: 6 - protein, 400 aa, Homo sapiens leukosialin; identical to SEQ ID NO: 2

SEQ ID NO: 7 - DNA, 2288 bp, Homo spaiens leukosialin
ggagcctcgggaggtggtggagtgacctggccccagtgctgcgtccttatcagccgagccggtcccagctcttgctcctgcctgtttgcctggaaatggccacgcttctccttctccttggggtgctggtggtaagcccagacgctctggggagcacaacagcagtgcagacacccacctccggagagcctttggtctctactagcgagcccctgagctcaaagatgtacaccacttcaataacaagtgaccctaaggccgacagcactggggaccagacctcagccctacctccctcaacttccatcaatgagggatcccctctttggacttccattggtgccagcactggttcccctttacctgagccaacaacctaccaggaagtttccatcaagatgtcatcagtgccccaggaaacccctcatgcaaccagtcatcctgctgttcccataacagcaaactctctaggatcccacaccgtgacaggtggaaccataacaacgaactctccagaaacctccagtaggaccagtggagcccctgttaccacggcagctagctctctggagacctccagaggcacctctggaccccctcttaccatggcaactgtctctctggagacttccaaaggcacctctggaccccctgttaccatggcaactgactctctggagacctccactgggaccactggaccccctgttaccatgacaactggctctctggagccctccagcggggccagtggaccccaggtctctagcgtaaaactatctacaatgatgtctccaacgacctccaccaacgcaagcactgtgcccttccggaacccagatgagaactcacgaggcatgctgccagtggctgtgcttgtggccctgctggcggtcatagtcctcgtggctctgctcctgctgtggcgccggcggcagaagcggcggactggggccctcgtgctgagcagaggcggcaagcgtaacggggtggtggacgcctgggctgggccagcccaggtccctgaggagggggccgtgacagtgaccgtgggagggtccgggggcgacaagggctctgggttccccgatggggaggggtctagccgtcggcccacgctcaccactttctttggcagacggaagtctcgccagggctccctggcgatggaggagctgaagtctgggtcaggccccagcctcaaaggggaggaggagccactggtggccagtgaggatggggctgtggacgccccagctcctgatgagcccgaagggggagacggggctgccccttaagtgtcggtgaatagtgaggctggaggccggaatctcagccagcctccagcaccttccctctcaccatcccactgccccctcgctcccatgtttccacccggcaccctgatcctcacccgaatctcctttttttttttcttttgagacagagtttcgctttgtcgcccaggctggagtgcaatgcacgatctcagttcactgcaacctctgcctcctaagttcaggcgattctcctgcctcagcttcccgagtaactgagattacaggcacccaccaccatgcccagctgcttttttgtatttttggtagagatggggtttcaccatgttggctaggctggtctcaaactcctgacctcaggtgatctacctgcctcagcctcccaaagtgctgagattacagacatgagcctccgcgccttgcctcctcacccacctcttcactctgaatcctcatgaggcttctcagccctggatttcctgctgccatcctcacccagcacccacaactagcgcctgggcagggcagggctggcacctctcaacgtctgtggactgaatgaataaaccctcctcttacaaatgccaaaattcattcagctttgatgataaacactgaggcccaatggcctttatcatctagggagtatgaagaatgagcaagaggctaactcagcgtgagttaccctgggaaaggaaagaagaaatggttcatataggacacacatagataccttcaagggtgtttgtagagttgtttcttaagtagttggtttccttcacagaaagttcttaaactcagaatatacccatccatgcaccccaccagcaatacaaagaccccaaacaaaaaattactatattcttaccctacgcatggcttcctcctcttcttgacgcttttcataatgtgcaaagtcatcaaagattgaggtggtatgcttgaaagtagcaattattttaagcacttgcttagctttttcaagggacg

SEQ ID NO: 8 - protein, 400 aa, Homo sapiens leukosialin; identical to SEQ ID NO: 2

SEQ ID NO: 9 - DNA, 5050 bp, Homo sapiens leukosialin (CD43)
cccccctgcagaatgggcaccccgttacctttctgagccactgtgcgcagaaaagagagcatgttggccaggctggtctcgaactcctgacctcaagtgatcagcctgccttacctcccaaagtcctgggattacaggcgtgaaccaccacgctcagcctctgaatactttgtactcaagccatttttcagtgctgtgtttgcagtgagcacacccgagggatgaagacacgtctccctgtgggaacctgggcttaccagggcccctagaggaggggaatctctcaagctcagagctctatggctgcggtgcaggcccactgtgtgcatggtgtcagtctgggcccttccatgttgcccccgtgggacttggggtaaggggaactgatgcaaacatcacgctgctgttgcttggtgtgagcaattaattcctgtggctctcacccaggagtctcatgtctttgggtcagacaaactcatcagcttgtagaaatggcacagtcccacgggcctgttagaatcttctattgtgcacatgttgctcttaaaatatacaaatcagttttgattttaaaaaattatttatttttttagtgataggagttttgctacgttgcccaggctggtttcaaactcttgggctcaggaggtcctcccactttggcctggactgccagcataatgtatcaccacacccgggactgattttcgtttttcaagaacaaaaaccaaaaacatacacaaaccgagagtcaaagcttgctaattagaggaaagtcaggaaatgggaaccattcaaagaagaaaatacccccacctcctactctcacctatccaaagacaattaggtgaatcccttagtagatatctttccagacggttttccatatagattcccatatctggccaggcgcggtggctcacacctgtaatcctagcgcttggggaggctgaggcggatggaccacctgaggtcaggagttcgagaccagcctgaccaacatggagaaacctcgtctctacgaaaaatacaaaattagccgggcacagtggtgcaagcctgtaatcccagctactcaggaggccgaggcaggagaattgcttgaacctaggaggcagacattgtgctgagccgagccaagatcatgccattgcactaaactccgccttaaaaaaaaaaaaaaagattcccacatctttactagtttgcagaaataagatcctagcatatgcagtgtgtaggaaccaccttggtttagccacgtctctgtgactgggggccactgtggtgacccccagctccccggacagagtcaagagctcaccagcctgcaaaggttttcacggcccccagccagactcgggggcttcctcttgccctgctacttcctgggagctctgagggcaggaaatggcgccactcagctcctggcctaacagcttggggaccacaaatgcaaaggaaaccaccctcccctcccacctcctcctctgcacccttgagttctcaggctcacattcccaccacccacctctgagcccagccctccctagcatcaccacttccatcccattcctcagccaagagccaggaatcctgattccagatcccacgcttccctgcctccctcaggtgagccccagacccccaggcaccccgctggcccctgaaggagcaggtgatggtgctgtcttcgcccagcagctgtgggagcaggcgggtggggcaggatggaggggtgggtggggtgggtggagccagggcccacttcctttccccttggggccctgtccttcccagtcttgccccagcctcgggaggtggtggagtgacctggccccagtgctgcgtccttatcagccgagccggtaagagggtgagacttggtggggtaggggcctcagtgggcctgggaatgtgcctgtggcttgaaaagactctgacaggttatgatgggaagagattgggagccattgggctgcacagggtcagggaaggccaggaggggctggtcactgctggaatctaagctgctgaggctggagggagcctcaggatggggctgatgggggagctgccagcatctgttcctctgtcatttctgataacagtaaaagccagcatggaaaaaaccgttaaaccgcaggttgggcctggccgttggcagggaagtgggcagaggggaggcccggccaggtcctccggcaactcccgcgtgttctgcttctccggctgcccacctgcaggtcccagctcttgctcctgcctgtttgcctggaaatggccacgcttctccttctccttggggtgctggtggtaagcccagacgctctggggagcacaacagcagtgcagacacccacctccggagagcctttggtctctactagcgagcccctgagctcaaagatgtacaccacttcaataacaagtgaccctaaggccgacagcactggggaccagacctcagccctacctccctcaacttccatcaatgagggatcccctctttggacttccattggtgccagcactggttcccctttacctgagccaacaacctaccaggaagtttccatcaagatgtcatcagtgccccaggaaacccctcatgcaaccagtcatcctgctgttcccataacagcaaactctctaggatcccacaccgtgacaggtggaaccataacaacgaactctccagaaacctccagtaggaccagtggagcccctgttaccacggcagctagctctctggagacctccagaggcacctctggaccccctcttaccatggcaactgtctctctggagacttccaaaggcacctctggaccccctgttaccatggcaactgactctctggagacctccactgggaccactggaccccctgttaccatgacaactggctctctggagccctccagcggggccagtggaccccaggtctctagcgtaaaactatctacaatgatgtctccaacgacctccaccaacgcaagcactgtgcccttccggaacccagatgagaactcacgaggcatgctgccagtggctgtgcttgtggccctgctggcggtcatagtcctcgtggctctgctcctgctgtggcgccggcggcagaagcggcggactggggccctcgtgctgagcagaggcggcaagcgtaacggggtggtggacgcctgggctgggccagcccaggtccctgaggagggggccgtgacagtgaccgtgggagggtccgggggcgacaagggctctgggttccccgatggggaggggtctagccgtcggcccacgctcaccactttctttggcagacggaagtctcgccagggctccctggcgatggaggagctgaagtctgggtcaggccccagcctcaaaggggaggaggagccactggtggccagtgaggatggggctgtggacgccccagctcctgatgagcccgaagggggagacggggctgccccttaagtgtcggtgaatagtgaggctggaggccgcaatctcagccagcctccagcaccttccctctcaccatcccactgccccctcgctcccatgtttccacccggcaccctgatcctcacccgaatctcctttttttttttcttttgagacagagtttcgctttgtcgcccaggctggagtgcaatgcacgatctcagttcactgcaacctctgcctcctaagttcaggcgattctcctgcctcagcttcccgagtaactgagattacaggcacccaccaccatgcccagctgcttttttgtatttttggtagagatggggtttcaccatgttggctaggctggtctcaaactcctgacctcaggtgatctacctgcctcagcctcccaaagtgctgagattacagacatgagcctccgcgccttgcctcctcacccacctcttcactctgaatcctcatgaggcttctcagccctggatttcctgctgccatcctcacccagcacccacaactagcgcctgggcagggcagggctggcacctctcaacgtctgtggactgaatgaataaaccctcctcatccacccctatttatctccatcaccatttccccctctttcttgttcctggaaacggctgctgagtctccatcggccaaacttatctgccctgtgatttctttgacaattctccttttcccccagaacccaccctgggttgaccagagtctgggaagaaggacaagagaacccggcaaactccctcctaggattaactttgtaaagcacccttgccctgtagctgcaagggctgtggaacctgggcagcccgcaaccacctttagctctgggccccccaggccagcctggagcatggctgggtggggccaccagcccatgctctcaggcgggcctgtgatctttcccagggcacatggactgtaggctggccctggcccacaccaccacactctccccagccatggacagaggcagccagaggcctcacggtttctcctccgagtttctggctgggtgtagttctcagaaaccccagtgcctgcgtgtgtccactcgtgggtgtggtttgtgtgcaagagctgaggatttggcgatgcttgggaggggtagttgtgggtacagacggtgtgggggtgggaagtggtgcagagactgaagagggtcaacctgggcatgggggacacagggactgctgagaacgtgcgtgtcatctttgctctgatggggtggacatagcagaaaatctaactctgtctgtagccccatacagaatgccagggtgagcacagtggctggtgcctttaatcccagcactttggaaagttgaggcaggaggatcgcttgagcccaggagttcgagtctgaagtgagctgtgattgcaccactgcacttcagcctgggcaacagagtgagcccctgtctcaaaaaagaaaagaaaaagaaagccaggcttcatggaaagatcgtatgtgtgacccaatatgagttcttcagctcagccatggtaatcccttccttgaagtctccatttctgcagtacacatgcatgt

SEQ ID NO: 10 - protein, 400 aa, Homo sapiens leukosialin (CD43); identical to SEQ ID NO: 2

SEQ ID NO: 11 - DNA, 1879 bp, Homo sapiens sialophorin (CD43); identical to SEQ ID NO: 1

SEQ ID NO: 12 - protein, 400 aa, Homo sapiens sialophorin (CD43); identical to SEQ ID NO: 2

SEQ ID NO: 13 - DNA, 6503 bp, Homo sapiens sialophorin (CD43)
aagcttgctaattagaggaaagtcaggaaatgggaaccattcaaagaagaaaatacccccacctcctactctcacctatccaaagacaattaggtgaatcccttagtagatatctttccagacggttttccatatagattcccatatctggccaggcgcggtggctcacacctgtaatcctagcgcttggggaggctgaggcggatggaccacctgaggtcaggagttcgagaccagcctgaccaacatggagaaacctcgtctctacgaaaaatacaaaattagccgggcacagtggtgcaagcctgtaatcccagctactcaggaggccgaggcaggagaattgcttgaacctaggaggcagacattgtgctgagccgagccaagatcatgccattgcactaaactccgccttaaaaaaaaaaaaaaaagattcccacatctttactagtttgcagaaataagatcctagcatatgcagtgtgtaggaaccaccttggtttagccacgtctctgtgactgggggccactgtggtgacccccagctccccggacagagtcaagagctcaccagcctgcaaaggttttcacggcccccagccagactcgggggcttcctcttgccctgctacttcctgggagctctgagggcaggaaatggcgccactcagctcctggcctaacagcttggggaccacaaatgcaaaggaaaccaccctcccctcccacctcctcctctgcacccttgagttctcaggctcacattcccaccacccacctctgagcccagccctccctagcatcaccacttccatcccattcctcagccaagagccaggaatcctgattccagatcccacgcttccctgcctccctcaggtgagccccagacccccaggcaccccgctggcccctgaaggagcaggtgatggtgctgtcttcgcccagcagctgtgggagcaggcgggtggggcaggatggaggggtgggtggggtgggtggagccagggcccacttcctttccccttggggccctgtccttcccagtcttgccccagcctcgggaggtggtggagtgacctggccccagtgctgcgtccttatcagccgagccggtaagagggtgagacttggtggggtaggggcctcagtgggcctgggaatgtgcctgtggcttgaaaagactctgacaggttatgatgggaagagattgggagccattgggctgcacagggtcagggaaggccaggaggggctggtcactgctggaatctaagctgctgaggctggagggagcctcaggatggggctgatgggggagctgccagcatctgttcctctgtcatttctgataacagtaaaagccagcatggaaaaaaccgttaaaccgcaggttgggcctggccgttggcagggaagtgggcagaggggaggcccggccaggtcctccggcaactcccgcgtgttctgcttctccggctgcccacctgcaggtcccagctcttgctcctgcctgtttgcctggaaatggccacgcttctccttctccttggggtgctggtggtaagcccagacgctctggggagcacaacagcagtgcagacacccacctccggagagcctttggtctctactagcgagcccctgagctcaaagatgtacaccacttcaataacaagtgaccctaaggccgacagcactggggaccagacctcagccctacctccctcaacttccatcaatgagggatcccctctttggacttccattggtgccagcactggttcccctttacctgagccaacaacctaccaggaagtttccatcaagatgtcatcagtgccccaggaaacccctcatgcaaccagtcatcctgctgttcccataacagcaaactctctaggatcccacaccgtgacaggtggaaccataacaacgaactctccagaaacctccagtaggaccagtggagcccctgttaccacggcagctagctctctggagacctccagaggcacctctggaccccctcttaccatggcaactgtctctctggagacttccaaaggcacctctggaccccctgttaccatggcaactgactctctggagacctccactgggaccactggaccccctgttaccatgacaactggctctctggagccctccagcggggccagtggaccccaggtctctagcgtaaaactatctacaatgatgtctccaacgacctccaccaacgcaagcactgtgcccttccggaacccagatgagaactcacgaggcatgctgccagtggctgtgcttgtggccctgctggcggtcatagtcctcgtggctctgctcctgctgtggcgccggcggcagaagcggcggactggggccctcgtgctgagcagaggcggcaagcgtaacggggtggtggacgcctgggctgggccagcccaggtccctgaggagggggccgtgacagtgaccgtgggagggtccgggggcgacaagggctctgggttccccgatggggaggggtctagccgtcggcccacgctcaccactttctttggcagacggaagtctcgccagggctccctggcgatggaggagctgaagtctgggtcaggccccagcctcaaaggggaggaggagccactggtggccagtgaggatggggctgtggacgccccagctcctgatgagcccgaagggggagacggggctgccccttaagtgtcggtgaatagtgaggctggaggccggaatctcagccagcctccagcaccttccctctcaccatcccactgccccctcgctcccatgtttccacccggcaccctgatcctcacccgaatctcctttttttttttcttttgagacagagtttcgctttgtcgcccaggctggagtgcaatgcacgatctcagttcactgcaacctctgcctcctaagttcaggcgattctcctgcctcagcttcccgagtaactgagattacaggcacccaccaccatgcccagctgcttttttgtatttttggtagagatggggtttcaccatgttggctaggctggtctcaaactcctgacctcaggtgatctacctgcctcagcctcccaaagtgctgagattacagacatgagcctccgcgccttgcctcctcacccacctcttcactctgaatcctcatgaggcttctcagccctggatttcctgctgccatcctcacccagcacccacaactagcgcctgggcagggcagggctggcacctctcaacgtctgtggactgaatgaataaaccctcctcatccacccctatttatctccatcaccatttccccctctttcttgttcctggaaacggctgctgagtctccatcggccaaacttatctgccctgtgatttctttgacaattctccttttcccccagaacccaccctgggttgaccagagtctgggaagaaggacaagagaacccggcaaactccctcctaggattaactttgtaaagcacccttgccctgtagctgcaagggctgtggaacctgggcagcccgcaaccacctttagctctgggccccccaggccagcctggagcatggctgggtggggccaccagcccatgctctcaggcgggcctgtgatctttcccagggcacatggactgtaggctggccctggcccacaccaccacactctccccagccatggacagaggcagccagaggcctcacggtttctcctccgagtttctggctgggtgtagttctcagaaaccccagtgcctgcgtgtgtccactcgtgggtgtggtttgtgtgcaagagctgaggatttggcgatgcttgggaggggtagttgtgggtacagacggtgtgggggtgggaagtggtgcagagactgaagagggtcaacctgggcatgggggacacagggactgctgagaacgtgcgtgtcatctttgctctgatggggtggacatagcagaaaatctaactctgtctgtagccccatacagaatgccagggtgagcacagtggctggtgcctttaatcccagcactttggaaagttgaggcaggaggatcgcttgagcccaggagttcgagtctgaagtgagctgtgattgcaccactgcacttcagcctgggcaacagagtgagcccctgtctcaaaaaagaaaagaaaaagaaagccaggcttcatggaaagatcgtatgtgtgacccaaatatgagttcttcagctcagccatggtaatcccttccttgaagtctccatttctgcagtacacatgcatgtgcgctctctctctctctctctctctcacacacacacacacacacacacacacgcgcgcgcgcgcgcgcgcgcgcgctctcctgcgaacagaggcagggggagaggggtttgccctggtctcggggactggtctggctggcgcttccccactgcacgtttccaggtttagtttgtctgtgtctcctcttccatcccaggggctgagccccttccatcctccaagaggaaccagtgagagtgagtgaaggaggggcctggagccagggacttcccctgtggggcctgggtggagaggggagaactcaatggtgctgcctttgagaccagcccagctacagcccaggagcacacatgggccagggcagttggtatttcccgaggacaaagaggaaattttcaaagaggaagttgttgagttagagcttgcggtggctgagagcagacaggttgacctgcaaaaaaagacaggggaggcatgtgagtgtgacagccctgctctgtggcctgggcaggagatgggggaaagggtcaggtgggggatgggctcgtgcagtgggagaggagacggagggagggagcgggaaggggcttgcttagtgggtgggaagagctgagctcggatggaaccagcttctaccagccaggctgggcacccactgggctgcatctggtggccttttctgattgctatttggactcactgcagctgcagaatgacagaggccatgtccaaaatcccttagagacactgttgtcttagagttgttaaaataagagcccccatatcaggtttagaaaatactgtcaccgaacgaacgtcgctgtcctcagctccacctccctttcctttgacagatatggttgttttctaagccaggactggttttagtcaggtcctgggcgaatcctgaaaaaaagaggtagtacgggtaaggaaggcacccaacagggctttcacaatccagaaaatatcaaaatataagtgttaaaagagaggcacaggccgggtgcggtggctcacgcctgtaatctcagcactttgggaggccaaggtgggcagatcatgaggtcaggagtttgagaccagcctggccaatatgatgaaaccccgtttctactaaaaatacaaaagttagccaggcatggtggtgtgctcctgtaatcccagctacttaggaggctgaggccagagaattgcttgaaccctggagtcagaggttgcagtgagccgggatcatgccactgtactccaggctgggtgacaaagtgagactgtctcaaaaaataaaaataaataaaataaataaaagagaggcacaaacagtgttatgaatgcaccaaggaaaatggtgcattcataactctcaggtgaagcctaccaagccatgcgtgtgtgcacatatgtgtgtacgtgtgcatgtgcgtgcgtgcatgtgcgtgcgtgcatgtgcctgtgtgtgtatgtgtgcacatgtgtgtgcgcatgtgtgtgtgtgcgcgcatgtgtgtgtgcatgcatgttctcccatgcatgtgtactgtggcaagggagactttgaggaagagattccagtggctgagcagaagggctcgcattgccctggcgaaaggttggaaggcttcacctgagagtgtgtcgtggcctttgtcatatccactgcttgattcctttctttaaaaattatttttattgttttctacatatgagaaccaccacacctggctaatttttgtattttttgtagagatggggtttcaccatgttgtcccggctggtctcaaactcccgggcacaagagatccacctgcctcagcctcccaaaatgctgggactataggcatgagccactgcacccagccactgcttcattcctggtggctgctgtgcctggcatgttgcagatcctccatgaatatgcatttgaatgaatgaatgaatgaatgaatgaatggagatgacgcctcagagattctttcttttgagatgaggtctcattctgtcacccagactagagggcagtggtgcaatcacagctcaccacagcctcaacctcctgggcctcccaagtagctgcgatcacaggtgtgcaccaacatgcccagctaattttttttttaatttttaatttgtacagacagggtcttgctgtgttgcccaggctggtctcgaactcctgggctcaagtggtcctcccacctaagctt

SEQ ID NO: 14 - protein, 400 aa, Homo sapiens sialophorin (CD43); identical to SEQ ID NO: 2

SEQ ID NO: 15 - DNA, 2745 bp, Homo sapiens heterogeneous nuclear ribonucleoprotein K
cggcagtctcgcgcggctactgcagcactggggtgtcagttgttggtccgacccagaacgcttcagttctgctctgcaaggatatataataactgattggtgtgcccgtttaataaaagaatatggaaactgaacagccagaagaaaccttccctaacactgaaaccaatggtgaatttggtaaacgccctgcagaagatatggaagaggaacaagcatttaaaagatctagaaacactgatgagatggttgaattacgcattctgcttcagagcaagaatgctggggcagtgattggaaaaggaggcaagaatattaaggctctccgtacagactacaatgccagtgtttcagtcccagacagcagtggccccgagcgcatattgagtatcagtgctgatattgaaacaattggagaaattctgaagaaaatcatccctaccttggaagagggcctgcagttgccatcacccactgcaaccagccagctcccgctcgaatctgatgctgtggaatgcttaaattaccaacactataaaggaagtgactttgactgcgagttgaggctgttgattcatcagagtctagcaggaggaattattggggtcaaaggtgctaaaatcaaagaacttcgagagaacactcaaaccaccatcaagcttttccaggaatgctgtcctcattccactgacagagttgttcttattggaggaaaacccgatagggttgtagagtgcataaagatcatccttgatcttatatctgagtctcccatcaaaggacgtgcacagccttatgatcccaatttttacgatgaaacctatgattatggtggttttacaatgatgtttgatgaccgtcgcggacgcccagtgggatttcccatgcggggaagaggtggttttgacagaatgcctcctggtcggggtgggcgtcccatgcctccatctagaagagattatgatgatatgagccctcgtcgaggaccacctccccctcctcccggacgaggcggccggggtggtagcagagctcggaatcttcctcttcctccaccaccaccacctagagggggagacctcatggcctatgacagaagagggagacctggagaccgttacgacggcatggttggtttcagtgctgatgaaacttgggactctgcaatagatacatggagcccatcagaatggcagatggcttatgaaccacagggtggctccggatatgattattcctatgcagggggtcgtggctcatatggtgatcttggtggacctattattactacacaagtaactattcccaaagatttggctggatctattattggcaaaggtggtcagcggattaaacaaatccgtcatgagtcgggagcttcgatcaaaattgatgagcctttagaaggatccgaagatcggatcattaccattacaggaacacaggaccagatacagaatgcacagtatttgctgcagaacagtgtgaagcagtatgcagatgttgaaggattctaatgcaagatattttttcttttttatagtgtgaagcagtattctggaaagtttttctaagactagtgaagaactgaaggagtcctgcatctttttttttttatctgcttctgtttaaaaagccaacattcctctgcttcataggtgttctgcatttgaggtgtagtgaaatctttgctgttcaccagatgtaatgttttagttccttacaaacagggttgggggggggaagggcgtgcaaaaactaacattgaaattttgaaacagcagcagagtgagtggattttatttttcgttattgttggtggtttaaaaaattccccccatgtaattattgtgaacaccttgctttgtggtcactgtaacatttggggggtgggacagggaggaaaagtaacaatagtccacatgtccctggcatctgttcagagcagtgtgcagaatgtaatgctcttttgtaagaaacgttttatgatttttaaaataaatttagtgaacctatttttggtggtcattttttttttaagacagtcattttaaaatggtggctgaatttcccaacccacccccaaactaaacactaagtttaattttcagctcctctgttggacatataagtgcatctcttgttggacataggcaaaataacttggcaaacttagttctggtgatttcttgatggtttggaagtctattgctgggaagaaattccatcatacatattcatgcttataataagctggggattttttgtttgtttttgcaaatgcttgcccctacttttcaacaattttctatgttagttgtgaagaactaaggtggggagcagtactacaagttgagtaatggtatgagtatataccagaattctgattggcagcaagtttattaatcagaataacacttggttatggaagtgactaatgctgaaaaaattgattatttttattagataatttctcacctatagacttaaactgtcaatttgctctagtgtcttattagttaaactttgtaaaatatatatatacttgtttttccattgtatgcaaattgaaagaaaaagatgtaccatttctctgttgtatgttggattatgtaggaatgtttgtgtacaattcaaaaaaaaaaaagatgaaaaaagttcctgtggatgttttgtgtagtatcttggcatttgtattgatagttaaaattcacttccaaataaataaaacacccatgatgctag

SEQ ID NO: 16 - protein, 463 aa, Homo sapiens heterogeneous nuclear ribonucleoprotein complex K
METEQPEETFPNTETNGEFGKRPAEDMEEEQAFKRSRNTDEMVELRILLQSKNAGAVIGKGGKNIKALRTDYNASVSVPDSSGPERILSISADIETIGEILKKIIPTLEEGLQLPSPTATSQLPLESDAVECLNYQHYKGSDFDCELRLLIHQSLAGGIIGVKGAKIKELRENTQTTIKLFQECCPHSTDRVVLIGGKPDRVVECIKIILDLISESPIKGRAQPYDPNFYDETYDYGGFTMMFDDRRGRPVGFPMRGRGGFDRMPPGRGGRPMPPSRRDYDDMSPRRGPPPPPPGRGGRGGSRARNLPLPPPPPPRGGDLMAYDRRGRPGDRYDGMVGFSADETWDSAIDTWSPSEWQMAYEPQGGSGYDYSYAGGRGSYGDLGGPIITTQVTIPKDLAGSIIGKGGQRIKQIRHESGASIKIDEPLEGSEDRIITITGTQDQIQNAQYLLQNSVKQYSGKFF

SEQ ID NO: 17 - DNA, 1144 bp, Homo sapiens Pur (pur-alpha)
cgactgaggcggcgggcggagcggcaggcggcggcggcgcggcagcggagcgcagcatcatggcggaccgagacagcggcagcgagcagggtggtgcggcgctgggttcgggcggctccctggggcaccccggctcgggctcaggctccggcgggggcggtggtggcggcgggggcggcggcggcagtggcggcggcggcggcggggccccaggggggctgcagcacgagacgcaggagctggcctccaagcgggtggacatccagaacaagcgcttctacctggacgtgaagcagaacgccaagggccgcttcctgaagatcgccgaggtgggcgcgggcggcaacaagagccgccttactctctccatgtcagtggccgtggagttccgcgactacctgggcgacttcatcgagcactacgcgcagctgggccccagccagccgccggacctggcccaggcgcaggacgagccgcgccgggcgctcaaaagcgagttcctggtgcgcgagaaccgcaagtactacatggatctcaaggagaaccagcgcggccgcttcctgcgcatccgccagacggtcaaccgggggcctggcctgggctccacgcagggccagaccattgcgctgcccgcgcaggggctcatcgagttccgtgacgctctggccaagctcatcgacgactacggagtggaggaggagccggccgagctgcccgagggcacctccttgactgtggacaacaagcgcttcttcttcgatgtgggctccaacaagtacggcgtgtttatgcgagtgagcgaggtgaagcccacctatcgcaactccatcaccgtgccctacaaggtgtgggccaagttcggacacaccttctgcaagtactcggaggagatgaagaagattcaagagaagcagagggagaagcgggctgcctgtgagcagcttcaccagcagcaacagcagcagcaggaggagaccgccgctgccactctgctactgcagggtgaggaagaaggggaagaagattgatcaaacagaatgaaacccccacacacacacacatgcatacacacacacacacagccacacacacagaaaatatactgtaaagaaagagagaaaataaaaagttaaaaagttaaaaa

SEQ ID NO: 18 - protein, 322 aa, Homo sapiens purine-rich element binding protein A (PURA)
MADRDSGSEQGGAALGSGGSLGHPGSGSGSGGGGGGGGGGGGSGGGGGGAPGGLQHETQELASKRVDIQNKRFYLDVKQNAKGRFLKIAEVGAGGNKSRLTLSMSVAVEFRDYLGDFIEHYAQLGPSQPPDLAQAQDEPRRALKSEFLVRENRKYYMDLKENQRGRFLRIRQTVNRGPGLGSTQGQTIALPAQGLIEFRDALAKLIDDYGVEEEPAELPEGTSLTVDNKRFFFDVGSNKYGVFMRVSEVKPTYRNSITVPYKVWAKFGHTFCKYSEEMKKIQEKQREKRAACEQLHQQQQQQQEETAAATLLLQGEEEGEED

SEQ ID NO: 19 - DNA, 22 bp, Synthetic oligonucleotide (CD43 PyRo SS)
gggcccacttcctttccccttg

SEQ ID NO: 20 - DNA, 16 bp, Synthetic oligonucleotide (CD43 PyRo SSUB)
  misc_feature (9)..(10): bromouracil
  misc_feature (13)..(15): bromouracil
  misc_feature (20)..(21): bromouracil
  misc_feature (23)..(23): biotin
gggcccacccccccgb

SEQ ID NO: 21 - DNA, 22 bp, Synthetic oligonucleotide (CD43 Mut-11)
gggcccacttccttcatatatg

SEQ ID NO: 22 - DNA, 20 bp, Synthetic oligonucleotide (NS-SS)
gagttagctcactcattagg

SEQ ID NO: 23 - DNA, 21 bp, Synthetic oligonucleotide(LUC-2)
atagccttatgcagttgctct

SEQ ID NO: 24 - DNA, 39 bp, Synthetic oligonucleotide (GeneRacer RNA Oligo)
  misc_feature (5)..(5): bromouracil
  misc_feature (21)..(21): bromouracil
  misc_feature (26)..(26): bromouracil
  misc_feature (31)..(31): bromouracil
  misc_feature (39)..(39): bromouracil
cgacggagcacgaggacacgacaggacgaaggagagaaa

SEQ ID NO: 25 - DNA, 54 bp, Synthetic oligonucleotide (GeneRacer Oligo dT Primer)
gctgtcaacgatacgctacgtaacggcatgacagtgtttttttttttttttttt

SEQ ID NO: 26 - DNA, 23 bp, Synthetic oligonucleotide (GeneRacer 5′ Primer)
cgactggagcacgaggacactga

SEQ ID NO: 27 - DNA, 27 bp, Synthetic oligonucleotide (GeneRacer 5′ Nested Primer)
ggacactgaccatggactgaaggagta

SEQ ID NO: 28 - DNA, 33 bp, Synthetic oligonucleotide (LUC-4)
cactacggtaggctgcgaaatgttcatactgtt

(19) United States
(12) Patent Application Publication
(10) Pub. No.: US 2006/0216231 A1
(43) Pub. Date: Sep. 28, 2006

(54) Methods for diagnosing and treating tumors and suppressing cd promoters

(76) Inventors: Carl Simon Shelley, Charlestown, MA (US); Omid C. Farokhzad, Chestnut Hill, MA (US)

Correspondence Address: WOLF GREENFIELD & SACKS, PC, FEDERAL RESERVE PLAZA, 600 ATLANTIC AVENUE, BOSTON, MA, 02210-2206, US

(21) Appl. No.: 10/528,948

(22) PCT Filed: Sep. 23, 2003
(86) PCT No.: PCT/US03/30213

§ 371 Date: Apr. 21, 2006

Related U.S. Application Data
(60) Provisional application No. 60/412,964, filed on Sep. 23, 2002.

(51) Int. Cl. A61K 51/00; A61K 48/00; A61K 39/395; C12Q 1/68; G01N 33/574

(52) U.S. Cl. 424/1.49; 435/6; 435/7.23; 424/155.1; 514/44

(57) ABSTRACT

Methods of treating tumors, reducing white blood cell numbers, and inhibiting CD promoters are provided.

### FIELD OF THE INVENTION

The invention relates to methods of treating tumors and methods of reducing the number of white blood cells. The invention also relates to methods of diagnosing ovarian tumors and assessing the prognosis and the progression of disease in patients with ovarian tumors. The invention also relates to methods of inhibiting CD promoters.

### BACKGROUND OF THE INVENTION

CD43 is a heavily glycosylated transmembrane molecule which plays a critical role in leukocyte activation and adhesion (Ostberg J R et al, (1998) Immunology Today,19:546-550; Remold-O'Donnell et al., (1990) Immunodeficiency Review, 2:151-174; Rosenstein et al., (1999) Immunology Research, 20:89-99). The importance of CD43 is demonstrated by two immunodeficiency diseases, Wiskott-Aldrich syndrome (WAS) and the early stages of HIV infection (Parkman et al., (1981) Lancet, ii:1387-1389; Remold-O'Donnell et al., (1984) Journal of Experimental Medicine, 159:1705-1723; Ardman et al., (1990) Journal of Experimental Medicine, 172:1151-1158; Lefebvre et al., (1994) Journal of Experimental Medicine, 180:1609-1617; Giordanengo et al., (1995) Blood, 86:2302-2311; Gallego et al., (2001) AIDS, 15:477-481). The etiology of both diseases involves the development of defects in CD43. Patients with WAS, which is an X-chromosome linked, recessive disorder, express defective CD43 on the surface of their T-lymphocytes. Affected males are subject to recurring opportunistic infections and do not respond to carbohydrate antigens reflecting defects in T-lymphocyte function. In addition, patients suffer from eczema and thrombocytopenia with platelets of reduced size and function. With regard to HIV infection, the finding that all affected individuals have circulating anti-CD43 antibodies has led to the suggestion that these auto-antibodies contribute to severe immunodeficiency (Giordanengo et al., (1995) Blood, 86:2302-2311).

CD43 is a large, abundant and highly charged trans-membrane molecule that is normally expressed on the surface of white blood cells. In this normal context of expression, CD43 acts as a barrier molecule preventing intercellular interactions and allowing blood cells to remain in the circulation.

CD43 is composed of 381 amino acids divided between a 235 residue extracellular region, a 23 residue transmembrane region and a 123 amino acid C-terminal intracellular region (Pallant et al., (1989) Proceedings of the National Academy of Sciences USA, 86:1328-1332; Shelley et al., (1989) Proceedings of the National Academy of Sciences USA, 86:2819-2823). The extracellular region contains approximately 84 sialylated O-linked carbohydrate units and appears by electron microscopy to be a rod-like structure extending 45 nm from the cell surface (Cyster et al., (1991) EMBO Journal, 10:893-902). Comparison of the rat, mouse and human sequences indicates that the intracellular domain has been highly conserved during evolution suggesting a critical function. The intracellular domain anchors CD43 to the cytoskeleton by binding actin, ezrin and moesin (Serrador et al., (1998) Blood, 91:4632-4644; Yonemura et al., (1998) Journal of Cell Biology, 140:885-895).

When leukocytes are at rest, CD43 maintains their circulation within the blood stream by preventing intercellular adhesion. This function is achieved by virtue of the size and strong negative charge of the extracellular domain (Brown et al., (1981) Nature, 289:456-460; Ardman et al., (1992) Proceedings of the National Academy of Sciences USA, 89:5001-5005; Manjunath et al., (1993) Journal of Immunology, 151:1528-1534; Dragone et al., (1995) Proceedings of the National Academy of Sciences USA, 92:626-630). During leukocyte activation CD43 expression is dramatically down-regulated allowing intercellular interactions mediated by molecules such as the β2 integrins. Intercellular interactions are also actively facilitated by CD43 which, due to changes in glycosylation, switches from being an anti-adhesion to a pro-adhesion molecule (Carlsson et al., (1986) Journal of Biological Chemistry, 261:12779-12786; Piller et al., (1988) Journal of Biological Chemistry, 263:15146-15150; Remold-O'Donnell et al., (1990) Journal of Immunology, 145:3372-3378. 22. Campanero et al., (1991) European Journal of Immunology, 21:3045-3048; Bazil et al., (1993) Proceedings of the National Academy of Sciences USA, 90:3792-3796; Ellies et al., (1994) Glycobiology, 4:885-893; Remold-O'Donnell et al. (1994) Journal of Immunology, 152:3595-3605; Tomlinson Jones et al., (1994) Journal of Immunology, 153:3426-3439; Ellies et al., (1996) Blood, 88:1725-1732; Weber et al., (1997) Immunobiology, 197:82-96).

The pro-adhesive function of CD43 is indicated by its identification as a counter receptor for galectin-1, ICAM-1 and the macrophage adhesion molecule sialoadhesin (Rosenstein et al., (1991) Nature, 354:233-235; Baum et al., (1995) Journal of Experimental Medicine, 181:877-887; Perillo et al., (1995) Nature, 378:736-739; Van den Berg et al., (2001) Journal of Immunology, 166:3637-3640). In addition, antibodies to CD43 have been shown to activate monocytes, B lymphocytes, dendritic, mast and natural killer cells (Vargas-Cortes et al., (1988) Scand Journal of Immunology, 27:661-671; Nong et al., (1989) Journal of Experimental Medicine, 170:259-267; Wiken et al., (1989) Scand Journal of Immunology, 29:353-361; Wiken et al., (1989) Scand Journal of Immunology, 29:363-370; Kuijpers et al., (1992) Journal of Immunology, 149:998-1003; Weber et al., (1994) Immunology, 82:638-644; Fanales-Belasio et al., (1997) Ad Exp Med Biology, 417:207-212). CD43 binds MHC class I molecules and activates T-lymphocytes in a manner independent of both the T-lymphocyte-receptor/CD3 complex and CD28 (Mentzer et al., (1987) Journal of Experimental Medicine, 165:1383-1392; Park et al., (1991) Nature, 350:706-709; Sperling et al., (1995) Journal of Experimental Medicine, 182:139-146; Stockl et al., (1996) Journal of Experimental Medicine, 184:1769-1779). The activation signals of this pathway are mediated through phosphorylation of the intracellular domain of CD43 and its physical interaction with the tyrosine kinases Fyn and Lck and the serine/threonine kinase STANK (Chatila et al., (1988) Journal of Immunology, 140:4308-4314; Silverman et al., (1989) Journal of Immunology, 142:4194-4200; Pedraza-Alva et al., (1996) Journal of Biological Chemistry, 271:27564-275 68; Wang et al., (2000) Cell Immunology, 205:34-39). Activation signals transduced by CD43 lead to phosphorylation of Shc, induction of the formation of a Shc/Grb2 complex, tyrosine phosphorylation of Vav, mitogen-activated protein kinase activation and nuclear translocation of ERK2 (Pedraza-Alva et al., (1998) Journal of Biological Chemistry, 273:14218-14224). Ultimately, these CD43-mediated signals induce the DNA binding activity of the transcription factors AP-1, NF-AT and NF-κB and activate the genes encoding interleukin-2, CD69 and CD40-L (Santana et al., (2000) Journal of Biological Chemistry, 275:31460-31468).

### SUMMARY OF THE INVENTION

The invention is based, in part, on the discovery that ovarian tumor cells abnormally express CD43 on their surfaces. The abnormal expression of CD43 on ovarian cells apparently contributes to the development of ovarian tumors. The invention provides methods of diagnosing ovarian tumors and assessing the prognosis and the progression of disease in patients with ovarian tumors. The invention is also based, in part, on the finding that CD43 inhibitors repress the CD43 promoter which mediates progression of tumors and promotes survival or proliferation of white blood cells. The invention provides methods to treat tumors and methods to reduce the number of white blood cells, as well as methods for inhibiting CD promoters.

According to one aspect of the invention, a method for characterizing an ovarian cell is provided. The method comprises determining the presence or absence of a CD43 molecule in an ovarian cell of a subject to characterize the ovarian cell.

A CD43 molecule refers to a CD43 nucleic acid or a CD43 protein. In some embodiments, the CD43 nucleic acid comprises the nucleic acid sequence of SEQ ID NO: 1 (GenBank Accession NO: NM_{—}003123), SEQ ID NO: 3 (GenBank Accession NO: BC012350), SEQ ID NO: 5 (GenBank Accession NO: X60502), SEQ ID NO: 7 (GenBank Accession NO: J04168), SEQ ID NO: 9 (GenBank Accession NO: M61827), SEQ ID NO: 11, (GenBank Accession NO: J04536), or SEQ ID NO: 13 (GenBank Accession NO: X52075), unique fragments and complements of the foregoing. In some embodiments a CD43 protein comprises the protein sequence of SEQ ID NO: 2, SEQ ID NO: 4, SEQ ID NO: 6, SEQ ID NO: 8, SEQ ID NO: 10, SEQ ID NO: 12, or SEQ ID NO: 14, unique fragments, and functional variants of the foregoing.

In some embodiments, the method for characterizing an ovarian cell is a diagnostic method wherein the presence of a CD43 molecule in the ovarian cell indicates that the subject has an ovarian tumor.

In some embodiments, the method for characterizing an ovarian cell is a prognostic method wherein the presence of a CD43 molecule in the ovarian cell indicates that the subject has a poor prognosis. A prognosis predicts the probable outcome of a disease or the prospect of recovery from disease. A poor prognosis, suggests that the outcome of the disease in the subject is poor or the subject has a low chance of recovery from the disease.

In some embodiments, the method for characterizing an ovarian cell is performed in vivo. In other embodiments, the method for characterizing an ovarian cell is performed in vitro.

Methods of detecting the presence of a CD43 molecule may be done in the presence of a CD43 binding molecule. One example of a CD43 binding molecule is a CD43 antibody.

In some embodiments, the CD43 antibody is bound to a label. In certain embodiments the label is selected from the group consisting of a fluorescent label, an enzyme label, a radioactive label, a nuclear magnetic resonance active label, a luminescent label, and a chromophore label.

A CD43 antibody may be a CD43 polyclonal antibody or a CD43 monoclonal antibody. One example of a CD43 monoclonal antibody is BS1. Other examples of CD43 monoclonal antibodies include but are not limited to: MEM-59, 84-3C1, Bra7G, DF-T1, 1G10, MT1, L10, L14, T2/53, B1-B6, L60, BL-GCE/G3, 6E5, 6F5, 10G7, G10-2, G19-1, DS 1.C1, L66, CBF.78, 148.1B6, 148.1C3, 148.3D4, 161.46, RDP.AD9, OH.01, HI165, and HI161.

According to another aspect of the invention, a method of treating a subject having or at risk of having a tumor is provided. The method comprises administering to a subject in need of such a treatment a CD43 inhibitor in an effective amount to treat the tumor.

In some embodiments, the CD43 inhibitor is a CD43 nucleic acid binding molecule. One example of a CD43 nucleic acid binding molecule is a heterogeneous nuclear protein K (hnRNP-K) molecule. A hnRNP-K molecule refers to a hnRNP-K nucleic acid or to a hnRNP-K protein. In some embodiments the hnRNP-K nucleic acid comprises the nucleic acid sequence of SEQ ID NO: 15, unique fragments and complements of the foregoing. In other embodiments a hnRNP-K comprises the protein sequence of SEQ ID NO: 16, unique fragments, and functional variants of the foregoing.

In some embodiments, the CD43 inhibitor is a transcription factor. In one embodiment, the transcription factor is a Purα molecule. A Purα molecule refers to a Purα nucleic acid or to a Purα protein. In some embodiments the Purα nucleic acid comprises the nucleic acid sequence of SEQ ID NO: 17, unique fragments and complements of the foregoing. In other embodiments a Purα comprises the protein sequence of SEQ ID NO: 18, unique fragments, and functional variants of the foregoing.

In some embodiments, the CD43 inhibitor is a CD43 antisense molecule.

In some embodiments, the CD43 inhibitor is a CD43 antibody. In some embodiments, the CD43 antibody is bound to a radioisotope. Some radioisotopes could emit a radiations. Others could emit β radiations. Still other radioisotopes could emit γ radiations. Examples of radioisotopes that could be used in this invention include: ²²⁵Ac, ²¹¹At, ²¹²Bi, ²¹³Bi, ¹⁸⁶Rh, ¹⁸⁸Rh, ¹⁷⁷Lu, ⁹⁰Y, ¹³¹I or ⁶⁷Cu, ¹²⁵I, ¹²³I or ⁷⁷Br.

In some embodiments, the CD43 antibody is bound to a therapeutic moiety. Examples of therapeutic moieties that may be bound to the CD43 antibody include but are not limited to drugs, toxins or fragments thereof, or enzymes or fragments thereof.

In some embodiments, the drug is a cytotoxic drug. Examples of the cytotoxic drugs include but are not limited to: calicheamicin, esperamicin, methotrexate, doxorubicin, melphalan, chlorambucil, ARA-C, vindesine, mitomycin C, cis-platinum, etoposide, bleomycin, and 5-fluorouracil.

CD43 antibodies may be polyclonal CD43 antibodies or monoclonal CD43 antibodies. Examples of monoclonal CD43 antibodies include but are not limited to BS 1, MEM-59, 84-3C1, Bra7G, DF-T1, 1G10, MT1, L10, L14, T2/53, B1-B6, L60, BL-GCE/G3, 6E5, 6F5, 10G7, G10-2, G19-1, DS 1.C1, L66, CBF.78, 148.1B6, 148.1B6, 148.1C3, 148.3D4, 161.46, RDP.AD9, OH.01, HI165, and HI161.

Tumors encompassed by the invention include but are not limited to benign and malignant solid tumors and benign and malignant non-solid tumors. Examples of solid tumors include but are not limited to: biliary tract cancer, brain cancer (including glioblastomas and medulloblastomas), breast cancer, cervical cancer, choriocarcinoma, colon cancer, endometrial cancer, esophageal cancer, gastric cancer, intraepithelial neoplasms, including Bowen's disease and Paget's disease, liver cancer, lung cancer, lymphomas, including Hodgkin's disease and lymphocytic lymphomas, neuroblastomas, oral cancer, including squamous cell carcinoma, ovarian cancer, including those arising from epithelial cells, stromal cells, germ cells and mesenchymal cells, pancreatic cancer, prostate cancer, rectal cancer, renal cancer including adenocarcinoma and Wilms tumor, sarcomas, including leiomyosarcoma, rhabdomyosarcoma, liposarcoma, fibrosarcoma and osteosarcoma, skin cancer, including melanoma, Kaposi's sarcoma, basocellular cancer and squamous cell cancer, testicular cancer, including germinal tumors (seminomas, and non-seminomas such as teratomas and choriocarcinomas), stromal tumors and germ cell tumors, and thyroid cancer, including thyroid adenocarcinoma and medullary carcinoma.

Examples of non-solid tumors include but are not limited to hematological neoplasms. A hematologic neoplasm includes, for example, lymphoid disorders, myeloid disorders, and AIDS associated leukemias.

Lymphoid disorders include but are not limited to acute lymphocytic leukemia and chronic lymphoproliferative disorders (e.g., lymphomas, myelomas, and chronic lymphoid leukemias). Lymphomas include Hodgkin's disease and non-Hodgkin's lymphoma. Chronic lymphoid leukemias include T cell chronic lymphoid leukemias and B cell chronic lymphoid leukemias.

Myeloid disorders include chronic myeloid disorders such as, for instance, chronic myeloproliferative disorders, myelodysplastic syndrome and acute myeloid leukemia. Chronic myeloproliferative disorders include but are not limited to angiogenic myeloid metaplasia, essential thrombocythemia, chronic myelogenous leukemia, polycythemia vera, and atypical myeloproliferative disorders. Atypical myeloproliferative disorders include, for example, atypical chronic myelogenous leukemia (CML), chronic neutrophilic leukemia, mast cell disease, and chronic eosinophilic leukemia.

A subject includes a mammal, such as a human, non-human primate, cow, horse, pig, sheep, goat, dog, cat, or rodent. In some preferred embodiments, the subject is a human.

In certain embodiments, the method of treatment further comprises administering one or more anti-tumor therapies. Antitumor therapies include surgery, radiation therapy, or chemotherapeutic agents

In one embodiment the tumor is an ovarian cancer. Ovarian tumors include serous cystoma, mucinous cystoma, endometrioid tumor, mesonephric tumor, transitional cell (Brenner) tumor, dermoid tumor, teratoma, fibroma, thecoma, luteoma, granulosa cell tumor, struma ovarii, dysgerminoma, malignant mixed mesodermal tumor, and carcinoma.

Examples of serous cystomas include serous benign cystadenomas, serous cystadenomas, and serous cystadenocarcinomas.

Examples of mucinous cystomas include mucinous benign cystadenomas, mucinous cystadenomas, and mucinous cystadenocarcinomas.

Examples of endometrioid tumors include endometrioid benign cysts and endometrioid adenocarcinomas.

Examples of mesonephric tumors include benign mesonephric tumors and mesonephric cystadenocarcinomas.

In some embodiments, the carcinoma is a clear cell carcinoma.

In some embodiments, the ovarian tumor has metastasized beyond the ovary.

In some embodiments of this invention treatment of ovarian tumor further comprises an anti-ovarian tumor therapy. Anti-ovarian tumor therapies include anti-ovarian tumor chemotherapeutic agents. Examples of anti-ovarian tumor chemotherapeutic agents include cisplatin (Platinol) or platinum-containing drug combinations. Other anti-ovarian tumor chemotherapeutic agents include one or more agents selected from the group consisting of: vinblastin, bleomycin, etoposide, hexamethylmelanine, gemcitabine, topotecan, isofosfamide, alkylating agents, progestational agents, and antiestrogens.

Examples of the alkylating agents include: Mechlorethamine (nitrogen mustard; HN2; Mustargen), Chlorambucil (Leukeran), Cyclophosphamide (Cytoxan), Melphalan (Alkeran), Thiotepa (triethylenethiophosphoramide), Busulfan (Myleran), Carmustine (BCNU), Lomustine (CCNU), Semustine (methyl-CCNU), Altretamine (hexamethylmelamine), Procarbazine (Matulane), Dacarbazine, and Carboplatin (Paraplatin).

Progestational agents include: progesterone, progesterone derivative, 17-ethinyl testosterone derivative, and 19-nortestosterone derivative.

Progesteron derivatives include: hydroxyprogesterone caproate, medroxyprogesterone acetate, and megestrol acetate.

One Example of 17-ethinyl testosterone derivative is Dimethisterone.

Examples of 19-nortestosterone derivatives include: Desogestrel, Norethynodrel, Lynestrenol, Norethindrone, Norethindrone acetate, Ethynodiol diacetate, and L-Norgestrel.

Antiestrogens include: Tamoxifen, anastrozole, and a combination of aminoglutethimide and hydrocortisone.

According to another aspect of the invention, a method for assessing the regression or progression of an ovarian tumor in a subject is provided. The method comprises the steps of measuring in a first ovarian cell obtained from the subject, the presence of a CD43 molecule, measuring in a second ovarian cell obtained from the subject the presence of a CD43 molecule, comparing the presence of the CD43 molecule in the first ovarian cell and the second ovarian cell, wherein a decrease in the presence of the CD43 molecule in the second ovarian cell compared to the first ovarian cell indicates regression of the ovarian tumor, and wherein an increase in the presence of the CD43 molecule in the second ovarian cell compared to the first ovarian cell indicates progression of the ovarian tumor.

In some embodiments, the CD43 molecule is a CD43 nucleic acid. In other embodiments, the CD43 molecule is a CD43 protein.

Measuring the presence of a CD43 molecule may be performed in vivo or in vitro.

In some embodiments, measuring the presence of a CD43 molecule is done in the presence of a CD43 binding molecule. Examples of CD43 binding molecules are listed above.

According to yet another aspect of the invention, a kit for diagnosing an ovarian tumor is provided. The kit comprises one or more CD43 binding molecules, one or more control agents, and instructions for the use of the CD43 binding molecules, and the control agents in the diagnosis of an ovarian tumor.

In some embodiments, the CD43 binding molecule binds a CD43 nucleic acid. In other embodiments, the CD43 binding molecule binds a CD43 protein.

The kit may be used for diagnosing an ovarian tumor in vivo or in vitro.

CD43 binding molecules that may be used in this aspect of the invention include the CD43 binding molecules described above.

In some embodiments, one or more control agents of the kit are bound to a substrate.

In still another aspect of the invention, a method of reducing the number of white blood cells in a subject is provided. The method involves administering to a subject in need of a reduction of the number of white blood cells, a CD43 inhibitor molecule in an effective amount to reduce the number of white blood cells in the subject.

In some embodiments, the CD43 inhibitor is a hnRNP-K molecule. In some embodiments, the CD43 inhibitor is a transcription factor such as a Purα molecule. In some embodiments, the CD43 inhibitor is a CD43 antisense molecule. Examples of CD43 antisense molecules are described above. In some embodiments, the CD43 inhibitor is a CD43 antibody. CD43 antibodies may be polyclonal CD43 antibodies or monoclonal CD43 antibodies. Examples of monoclonal CD43 antibodies are listed above.

Examples of subjects in need of a reduction in the number of white blood cells include subjects with white blood cell disorders such as acute leukemias, chronic leukemias, and lymphomas, and subjects who are about to undergo, are undergoing, or have undergone a bone marrow, organ, cellular, or material transplant.

In some embodiments, the method involves administering a CD43 inhibitor with a second therapy to reduce the number of white blood cells.

In yet another aspect of the invention, a method of inhibiting a CD promoter is provided. The method involves contacting the CD promoter. with a CD43 inhibitor in an amount effective to inhibit the CD promoter.

CD43 inhibitors are described above.

In some embodiments of the invention the CD promoter is inhibited in vivo. In other embodiments the CD promoter is inhibited in vitro.

Examples of CD promoters inhibited by a CD43 inhibitor include but are not limited to: CD43, CD11a, CD11b, CD11c, and CD11d. In some embodiments CD11C is specifically excluded.

These and other aspects of the invention, as well as various advantages and utilities, will be more apparent in reference to the following detailed description of the invention. Each of the limitations of the invention can encompass various embodiments of the invention. It is therefore, anticipated that each of the limitations involving any one element or combination of elements can be included in each aspect of the invention.

### BRIEF DESCRIPTION OF THE DRAWINGS

FIG. 1 shows down-regulation of CD43 mRNA during K562 activation.

FIG. 2 depicts repression of the CD43 promoter during K562 activation.

FIG. 3 demonstrates in vitro binding of recombinant hnRNP-K binds to the +18/+39 region of the CD43 promoter.

FIG. 4 depicts determination of the molecular mass of the Jurkat nuclear factor which binds the +18/+39 region of the CD43 promoter.

FIG. 5 shows repression by hnRNP-K of the CD43 promoter present within an extrachromosomal plasmid.

FIG. 6 shows repression by hnRNP-K of the CD43 promoter present within chromosomal DNA.

FIG. 7 is a Southern blot analysis of the transcript produced from the CD43 promoter linked to luciferase and integrated within the K562 genome.

FIG. 8 shows that hnRNP-K and Purα act together to repress the CD43 promoter.

FIG. 9 demonstrates a putative secondary structure of the CD43 promoter.

FIG. 10 shows the detection of CD43 expression in ovarian cancer specimens by immunocytochemistry.

### DETAILED DESCRIPTION OF THE INVENTION

The present invention is based, in part, on the discovery that CD43 plays a role in the diagnosis and treatment of tumors. One aspect of the present invention relates to a method of characterizing an ovarian cell based on determining the presence or absence of a CD43 molecule in the ovarian cell obtained from a subject wherein the presence of the CD43 molecule indicates that the subject has an ovarian tumor.

Methods for determining the presence or absence of a CD43 molecule in an ovarian cell are provided. The invention is directed to determining the presence or absence of a CD43 molecule in a ovarian cell and not on a non-ovarian cell that has metastasized to the ovary. In some embodiments, the method of determining the presence or absence of a CD43 molecule is performed in the presence of a CD43 binding molecule. This method involves providing a CD43 binding molecule which binds to a CD43 molecule of an ovarian cell. The cell is contacted with the CD43 binding molecule under conditions effective to permit binding of the CD43 binding molecule to the CD43 molecule of the cell or portions thereof. The CD43 binding molecule may be bound to a label that permits the detection of the ovarian cell or portions thereof upon binding of CD43 binding molecule to the ovarian cell or portions thereof. The presence of a CD43 molecule is detected by the detection of the label.

In some embodiments, the contact between the CD43 binding molecule and the ovarian cell is carried out in vivo in a living mammal and involves administering the CD43 binding molecule to the mammal under conditions that permit binding of the CD43 binding molecule to a CD43 molecule. Such administration can be carried out by any suitable method known to one of ordinary skill in the art.

In other embodiments, the contact between the CD43 binding molecule and the ovarian cell is carried out in vitro and involves contacting the CD43 binding molecule with an ovarian cell obtained from a mammal under conditions that permit binding of the CD43 binding molecule to a CD43 molecule. Such contacting can be carried out by any suitable method known to one of ordinary skill in the art. Other methods involve isolation of a cellular fraction(s), i.e., proteins or nucleic acids from the cell and contacting the fraction with a CD43 binding molecule.

As used herein, a “CD43 binding molecule” is a molecule that selectively binds to a CD43 molecule. The CD43 binding molecule binds to a CD43 nucleic acid, and /or unique fragments thereof, or to a CD43 protein and /or unique fragments thereof. Such CD43 binding molecules may be used to selectively bind to a CD43 molecule in a cell (in vivo or ex vivo) for imaging and therapeutic applications in which, for example, the binding molecule is tagged with a detectable label and/or a toxin for targeted delivery to the CD43 molecule.

In some embodiments, the CD43 binding molecule is a CD43 binding peptide or a CD43 antibody, e.g., an Fab or F(ab)₂ fragment of a CD43 antibody, or an antigen binding fragment of a CD43 antibody. Typically, the antigen binding fragment includes a CDR3 region that is selective for the CD43 molecule. Any of the various types of antibodies can be used for this purpose, including, for example, monoclonal antibodies, polyclonal antibodies, humanized antibodies and chimeric antibodies.

Thus, the invention provides agents which bind to CD43 molecules, and in certain embodiments to unique fragments of the CD43 molecule. Such binding partners can be used in screening assays to determine the presence or absence of a CD43 molecule and in purification protocols to isolate such CD43 molecules. Likewise, such binding partners can be used to selectively target drugs, toxins or other molecules to cells which express CD43 molecules. In this manner, cells present in solid or non-solid tumors which express CD43 molecules can be treated with cytotoxic compounds. Such agents also can be used to inhibit the native activity of the CD43 molecules, for example, by binding to such polypeptides, to further characterize the functions of these molecules.

Thus, a CD43 antibody is an antibody or fragment thereof having the ability to selectively bind to CD43 molecules, and to unique fragments thereof. Antibodies include polyclonal, monoclonal, and chimeric antibodies, prepared, for example, according to conventional methodology. Examples of CD43 antibodies include but are not limited to BS1, MEM-59, 84-3C1, Bra7G, DF-T1, IG10, MT1, L10, L14, T2/53, B1-B6, L60, BL-GCE/G3, 6E5, 6F5, 10G7, G10-2, G19-1, DS 1.Cl, L66, CBF.78, 148.1B6, 148.1C3, 148.3D4, 161.46, RDP.AD9, OH.01, HI165, and HI161.

Other CD43 antibodies may be prepared by any of a variety of methods, including administering protein, fragments of protein, cells expressing the protein or fragments thereof and the like to an animal to induce polyclonal antibodies. The production of monoclonal antibodies is according to techniques weil known in the art. Such antibodies may be used, for example, to identify tissues expressing protein or to purify protein. Antibodies also may be coupled to specific labeling agents for imaging or to antitumor agents, including, for example, methotrexate, radioiodinated compounds, toxins such as ricin, other cytostatic or cytolytic drugs, and so forth.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

The term “antigen-binding fragment” of an antibody as used herein, refers to one or more portions of an antibody that retain the ability to specifically bind to an antigen (e.g., CD43 molecule). It has been shown that the antigen-binding function of an antibody can be performed by fragments of a full-length antibody. Examples of binding fragments encompassed within the term “antigen-binding fragment” of an antibody include (i) a Fab fragment, a monovalent fragment consisting of the V_{L}, V_{H}, C_{L} and C_{H}1 domains; (ii) a F(ab′)₂ fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546) which consists of a V_{H} domain; and (vi) an isolated complementarity determining region (CDR). Furthermore, although the two domains of the Fv fragment, V and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional procedures, such as proteolytic fragmentation procedures, as described in J. Goding, Monoclonal Antibodies: Principles and Practice, pp 98-118 (N.Y. Academic Press 1983), which is hereby incorporated by reference as well as by other techniques known to those with skill in the art. The fragments are screened for utility in the same manner as are intact antibodies.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

The non-CDR regions of a mammalian antibody may be replaced with similar regions of nonspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. Thus, for example, PCT International Publication Number WO 92/04381 teaches the production and use of humanized murine RSV antibodies in which at least a portion of the murine FR regions have been replaced by FR regions of human origin. Such antibodies, including fragments of intact antibodies with antigen-binding ability, are often referred to as “chimeric” antibodies.

Thus, as will be apparent to one of ordinary skill in the art, the methods of the invention involve the use of F(ab′)₂, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies. Thus, the invention involves polypeptides of numerous size and type that bind specifically to a CD43 molecule. These polypeptides may be derived also from sources other than antibody technology. For example, such polypeptide binding agents can be provided by degenerate peptide libraries which can be readily prepared in solution, in immobilized form or as phage display libraries. Combinatorial libraries also can be synthesized of peptides containing one or more amino acids. Libraries further can be synthesized of peptides and non-peptide synthetic moieties.

The antibodies useful in the methods of the invention encompass various antibody isotypes, such as IgG1, IgG2, IgG3, IgG4, IgM, IgA1, IgA2, IgAsec, IgD, IgE. As used herein, “isotype” refers to the antibody class (e.g. IgM or IgGI) that is encoded by heavy chain constant region genes. The antibodies can be full length or can include only an antigen-binding fragment such as the antibody constant and/or variable domain of IgG1, IgG2, IgG3, IgG4, IgM, IgA1, IgA2, IgAsec, IgD or IgE or could consist of a Fab fragment, a F(ab′)₂ fragment, and a Fv fragment.

The antibodies useful according to the present invention can be polyclonal, monoclonal, or a mixture of polyclonal and monoclonal antibodies. The antibodies can be produced by a variety of techniques well known in the art. Procedures for raising polyclonal antibodies are well known. For example, anti-CD43 polyclonal antibodies may be raised by administering a CD43 molecule subcutaneously to New Zealand white rabbits which have first been bled to obtain pre-immune serum. The CD43 molecule can be injected at a total volume of 100 μl per site at six different sites, typically with one or more adjustments. The rabbits are then bled two weeks after the first injection and periodically boosted with the same antigen three times every six weeks. A sample of serum is collected 10 days after each boost. Polyclonal antibodies are recovered from the serum, preferably by affinity chromatography using a CD43 molecule to capture the antibody. This and other procedures for raising polyclonal antibodies are disclosed in E. Harlow, et. al., editors, Antibodies: A Laboratory Manual (1988), which is hereby incorporated by reference.

Monoclonal antibody production may be effected by techniques which are also well known in the art. The term “monoclonal antibody,” as used herein, refers to a preparation of antibody molecules of single molecular composition. A monoclonal antibody displays a single binding specificity and affinity for a particular epitope. The process of monoclonal antibody production involves obtaining immune somatic cells with the potential for producing antibody, in particular B lymphocytes, which have been previously immunized with the antigen of interest either in vivo or in vitro and that are suitable for fusion with a B-cell myeloma line.

An antibody can be linked to a detectable marker, an antitumor agent or an immunomodulator. Antitumor agents can include cytotoxic agents and agents that act on tumor neovasculature. Detectable markers include, for example, radioactive or fluorescent markers. Cytotoxic agents include cytotoxic radionuclides, chemical toxins or chemotherapeutic agents and protein toxins.

The cytotoxic radionuclide or radiotherapeutic isotope may be an alpha-emitting isotope such as ²²⁵Ac, ²¹¹At, ²¹²Bi, or ²¹³Bi. Alternatively, the cytotoxic radionuclide may a beta-emitting isotope such as 186Rh, 188Rh, ¹⁷⁷Lu, ⁹⁰Y, ¹³¹I or ⁶⁷Cu. Further, the cytotoxic radionuclide may emit low energy electrons and include the isotopes ¹²⁵I, ¹²³I or ⁷⁷Br.

Suitable chemical toxins or chemotherapeutic agents include, for example, members of the enediyne family of molecules, such as calicheamicin and esperamicin. Chemical toxins can also be taken from the group consisting of methotrexate, doxorubicin, melphalan, chlorambucil, ARA-C, vindesine, mitomycin C, cis-platinum, etoposide, bleomycin and 5-fluorouaracil. Of course, combinations of the various toxins could also be coupled to one antibody molecule thereby accommodating variable cytotoxicity. Other chemotherapeutic agents are known to those skilled in the art.

The coupling of one or more toxin molecules to the CD43 antibody is envisioned to include many chemical mechanisms, for instance covalent binding, affinity binding, intercalation, coordinate binding, and complexation. The toxic compounds used to prepare the anti-CD43 imtnunotoxins are attached to the CD43 antibodies thereof by standard protocols known in the art.

The covalent binding can be achieved either by direct condensation of existing side chains or by the incorporation of external bridging molecules. Many bivalent or polyvalent agents are useful in coupling protein molecules to other proteins, peptides or amine functions, etc. For example, the literature is replete with coupling agents such as carbodiimides, diisocyanates, glutaraldehyde, diazobenzenes, and hexamethylene diamines. This list is not intended to be exhaustive of the various coupling agents known in the art but, rather, is exemplary of the more common coupling agents.

In some embodiments, it is contemplated that one may wish to first derivatize the antibody, and then attach the toxin component to the derivatized product. Suitable cross-linking agents for use in this manner include, for example, SPDP N-succinimidyl-3-(2-pyridyldithio)propionate), and SMPT, 4-succinimidyl-oxycarbonyl-methyl-(2-pyridyldithio)toluene.

In addition, protein toxins can be fused to the CD43 antibody by genetic methods to form a hybrid immunotoxin fusion protein. To make a fusion immunotoxin protein in accordance with the invention, a nucleic acid molecule is generated that encodes a CD43 antibody, a fragment of CD43 antibody, a single chain CD43 antibody, or a subunit of a CD43 antibody linked to a protein toxin. Such fusion proteins contain at least a targeting agent (e.g., CD43 antibody subunit) and a toxin, operatively attached. The fusion proteins may also include additional peptide sequences, such as peptide spacers which operatively attach the targeting agent and toxin compound, as long as such additional sequences do not appreciably affect the targeting or toxin activities of the fusion protein. The two proteins can be attached by a peptide linker or spacer, such as a glycine-serine spacer peptide, or a peptide hinge, as is well known in the art. Thus, for example, the C-terminus of a CD43 antibody can be fused to the N-terminus of the protein toxin molecule to form an immunotoxin that retains the binding properties of the CD43 antibody. Other fusion arrangements will be known to one of ordinary skill in the art.

To express the fusion immunotoxin, the nucleic acid encoding the fusion protein is inserted into an expression vector in accordance with standard methods, for stable expression of the fusion protein, preferably in mammalian cells, such as CHO cells. The fusion protein can be isolated and purified from the cells or culture supernatant using standard methodology, such as a CD43 affinity column.

Radionuclides typically are coupled to an antibody by chelation. For example, in the case of metallic radionuclides, a bifunctional chelator is commonly used to link the isotope to the antibody or other protein of interest. Typically, the chelator is first attached to the antibody, and the chelator-antibody conjugate is contacted with the metallic radioisotope. A number of bifunctional chelators have been developed for this purpose, including the diethylenetriamine pentaacetic acid (DTPA) series of amino acids described in U.S. Pat. Nos. 5,124,471, 5,286,850 and 5,434,287, which are incorporated herein by reference. As another example, hydroxamic acid-based bifunctional chelating agents are described in U.S. Pat. No. 5,756,825, the contents of which are incorporated herein. Another example is the chelating agent termed p-SCN-Bz-HEHA (1,4,7,10,13,16-hexaazacyclo-octadecane-N,N′,N″,N′″,N″″,N′″″-hexaacetic acid) (Deal et al., J Med. Chem. 42:2988, 1999), which is an effective chelator of radiometals such as ²²⁵Ac.

In addition, the CD43 antibodies can be used in immunofluorescence techniques to examine human tissue, cell and bodily fluid specimens. In one protocol, slides containing cryostat sections of frozen, unfixed tissue biopsy samples or cytological smears are air dried, formalin or acetone fixed, and incubated with the monoclonal antibody preparation in a humidified chamber at room temperature. The slides are then washed and further incubated with a preparation of a secondary antibody directed against the monoclonal antibody, usually some type of anti-mouse immunoglobulin if the monoclonal antibodies used are derived from the fusion of a mouse spleen lymphocyte and a mouse myeloma cell line. This secondary antibody is tagged with a compound, for instance rhodamine or fluorescein isothiocyanate, that fluoresces at a particular wavelength, The staining pattern and intensities within the sample are then determined by fluorescent light microscopy and optionally photographically recorded.

As another alternative, computer enhanced fluorescence image analysis or flow cytometry can be used to examine tissue specimens or cells, i.e., single cell preparations from aspiration biopsies of tumors using CD43 antibodies. The CD43 antibodies are particularly useful in quantitation of live tumor cells, i.e., single cell preparations from biopsies of tumors by computer enhanced fluorescence image analyzer or with a flow cytometer. The antibodies are particularly useful, for example, in such assays to differentiate benign from malignant ovarian tumors since the CD43 molecule to which the CD43 antibody binds is expressed in increased amounts by malignant tumors as compared to benign ovarian tumors. The percent CD43 positive cell population, alone or in conjunction with determination of other attributes of the cells (e.g., DNA ploidy of these cells), may, additionally, provide very usefill prognostic information by providing an early indicator of disease progression.

The antibodies may also be used in combination with other known antibodies to provide additional information regarding the malignant phenotype of a tumor.

The method of the present invention can be used to screen patients for diseases associated with the presence of tumors. Alternatively, it can be used to identify the recurrence of such diseases, particularly when the disease is localized in a particular biological material of the patient. Using the method of the present invention, this recurrence can be detected by administering a short range radiolabeled antibody to the mammal and then detecting the label.

Alternatively, the contacting step can be carried out in a biological sample. When the contacting is carried out in a biological sample, it is preferred that the biological sample recognize substantially no antigens other than CD43. Thus, the biological samples and methods of the present invention can be used to determine the effectiveness of a tumor treatment protocol by monitoring the level of CD43 in a biological sample.

The CD43 antibodies or antigen-binding fragments thereof can also be utilized in in vivo therapy of tumors. As used herein, therapy includes prophylaxis, which encompasses preventing or delaying the developement or the progression of a tumor.

The antibodies can be used alone or covalently attached, either directly or via linler, to a compound which kills and/or inhibits proliferation of the tumor following administration and localization of the conjugates. When the antibody is used by itself, it may mediate tumor destruction by complement fixation or antibody-dependent cellular cytotoxicity. Alternatively, the antibody may be administered in combination with a chemotherapeutic drug to cause synergistic therapeutic effects (3aslya and Mendelsohn, 1994 Breast Cancer Res. and Treatment 29:127-138). A variety of different types of substances can be directly conjugated to the antibody for therapeutic uses, including radioactive metal and non-metal isotopes, chemotherapeutic drugs, toxins, etc. as described above and known in the art (see, e.g., Vitetta and Uhr, 1985, Annu. Rev. Immunol. 3:197).

The CD43 antibodies thereof can also be administered together with complement. Accordingly, within the scope of the invention are compositions comprising antibodies or antigen-binding fragments thereof and serum or complement. These compositions are advantageous in that the complement is located in close proximity to the human antibodies or antigen-binding fragments thereof. Alternatively, the antibodies or antigen-binding fragments thereof of the invention and the complement or serum can be administered separately.

The CD43 antibodies can be administered with one or more immunostimulatory agents to induce or enhance an immune response, such as IL-2 and immunostimulatory oligonucleotides (e.g., those containing CpG motifs). Preferred immunostimulatory agents stimulate specific arms of the immune system, such as natural Iiller (NK) cells that mediate antibody-dependent cell cytotoxicity (ADCC).

Antigens, such as the CD43 molecule, can be administered with one or more adjuvants to induce or enhance an immune response. An adjuvant is a substance which potentiates the immune response. Adjuvants of many kinds are well known in the art. Specific examples of adjuvants include monophosphoryl lipid A (MPL, SmithKline Beecham); saponins including QS21 (SmithKline Beecham); immunostimulatory oligonucleotides (e.g., CpG oligonucleotides described by Kreig et al., Nature 374:546-9, 1995); incomplete Freund's adjuvant; complete Freund's adjuvant; montanide; vitamin E and various water-in-oil emulsions prepared from biodegradable oils such as squalene and/or tocopherol.

Other agents which stimulate the immune response of the subject to a CD43 molecule can also be administered to the subject. For example, cytokines are also useful in vaccination protocols as a result of their lymphocyte regulatory properties. Many cytoldnes useful for such purposes will be known to one of ordinary skill in the art, including interleukin-2 (IL-2); IL-12, which has been shown to enhance the protective effects of vaccines (see, e.g., Science 268: 1432-1434, 1995); GM-CSF; IL-18 and the like. Thus, cytokines can be administered in conjunction with antibodies, antigens, and/or adjuvants to increase an immune response.

The antibodies or antigen-binding fragments thereof can be used in conjunction with other therapeutic treatment modalities. Such other treatments include surgery, radiation, cryosurgery, thermotherapy, hormone treatment, chemotherapy, vaccines, and other immunotherapies.

Also within the scope of the invention are kits to measure the levels of a CD43 molecule and instructions for use. The kits could be used for detecting (diagnosing, prognosis, or monitoring) a tumor. The kits can further contain a least one additional reagent, such as complement, or one or more additional antibodies of the invention (e.g., an antibody having a complementary activity which binds to an epitope in the CD43 molecule distinct from the first antibody).

Kits containing the CD43 antibodies can be prepared for in vitro diagnosis, prognosis and/or monitoring a tumor by the immunohistological, immunocytological and immunoserological methods described above. The components of the kits can be packaged either in aqueous medium or in lyophilized form. When the antibodies are used in the kits in the form of conjugates in which a label moiety is attached, such as an enzyme or a radioactive metal ion, the components of such conjugates can be supplied either in fully conjugated form, in the form of intermediates or as separate moieties to be conjugated by the user of the kit.

A kit may comprise a carrier being compartmentalized to receive in close confinement therein one or more container means or series of container means such as test tubes, vials, flasks, bottles, syringes, or the like. A first of said container means or series of container means may contain one or more CD43 antibodies or a CD43 molecule. A second container means or series of container means may contain a label or linker-label intermediate capable of binding to the primary CD43 antibody (or fragment thereof).

Kits for use in in vivo tumor localization and therapy method containing the CD43 antibodies thereof conjugated to other compounds or substances can be prepared. The components of the kits can be packaged either in aqueous medium or in lyophilized form. When the antibodies or antigen-binding fragments thereof are used in the kits in the form of conjugates in which a label or a therapeutic moiety is attached, such as a radioactive metal ion or a therapeutic drug moiety, the components of such conjugates can be supplied either in fully conjugated form, in the form of intermediates or as separate moieties to be conjugated by the user of the kit.

It should be understood that kits could be assembled as to provide convenient access and use in clinical settings. Individuals skilled in the art can readily modify packaging to suit individual needs.

The invention in some aspects involves a method of treating a subject having or at risk of having a tumor. The method involves administering to a subject in need of such treatment a CD43 inhibitor in an effective amount to treat the tumor.

As used herein, a “CD43 inhibitor” is a molecule that inhibits (reduces or eliminates) the expression of a CD43 molecule or inhibits the function of a CD43 molecule.

As used herein, a “CD43 molecule” refers to a CD43 nucleic acid or to a CD43 protein. A CD43 nucleic acid refers to a nucleic acid which encodes a “CD43 protein.” In some embodiments the CD43 nucleic acid is selected from the group consisting of:

(a) nucleic acids which hybridize under stringent conditions to a nucleic acid having a nucleotide sequence set forth as SEQ ID NO: 1 (GenBank Accession NO: NM_{—}003123), SEQ ID NO: 3 (GenBank Accession NO: BC012350), SEQ ID NO: 5 (GenBank Accession NO: X60502), SEQ ID NO: 7 (GenBank Accession NO: J04168), SEQ ID NO: 9 (GenBank Accession NO: M61827), SEQ ID NO: 11, (GenBank Accession NO: J04536), or SEQ ID NO: 13 (GenBank Accession NO: X52075) and which code for a CD43 protein,

(b) deletions, additions and substitutions of the nucleic acids of (a), and

(c) nucleic acids that differ from the nucleic acids of (a) or (b) in codon sequence due to the degeneracy of the genetic code.

As used herein a “CD43 protein” refers to a protein having the activity of a native CD43 protein such as that described by SEQ ID NO: 2, SEQ ID NO: 4, SEQ ID NO: 6, SEQ ID NO: 8, SEQ ID NO: 10, SEQ ID NO: 12, or SEQ ID NO: 14, as well as unique fragments, functionally active fragments or variants thereof, provided that the functional fragments or variants exhibit a CD43 functional activity. The term protein as used herein encompasses the terms polypeptide and peptide As used herein, a CD43 functional activity refers, for example, to the ability of a CD43 protein to prevent intercellular adhesions.

The invention embraces variants of the CD43 proteins described herein. As used herein, a “variant” of a CD43 protein is a protein which contains one or more modifications to the primary amino acid sequence of a CD43 protein. Modifications which create a CD43 protein variant can be made to a CD43 protein 1) to produce, increase, reduce, or eliminate activity of the CD43 protein; 2) to enhance a property of the CD43 protein, such as protein stability in an expression system or the stability of protein-protein binding; or 3) to provide a novel activity or property to a CD43 protein. Modifications to a CD43 protein are typically made to the CD43 nucleic acid which encodes the CD43 protein, and can include deletions, point mutations, truncations, amino acid substitutions and additions of amino acids or non-amino acid moieties. Alternatively, modifications can be made directly to the protein, such as by cleavage, addition of a linker molecule, addition of a detectable moiety, such as biotin, addition of a fatty acid, and the like. Modifications also embrace fusion proteins comprising all or part of the CD43 amino acid sequences.

One of skill in the art will be familiar with methods for predicting the effect on protein conformation of a change in protein sequence, and can thus “design” a variant protein (e.g. CD43) according to known methods. One example of such a method is described by Dahiyat and Mayo in Science 278:82-87, 1997, whereby proteins can be designed de novo. The method can be applied to a known protein to vary only a portion of the protein sequence. By applying the computational methods of Dahiyat and Mayo, specific variants of a protein can be proposed and tested to determine whether the variant retains a desired conformation.

In general, functional variants of a protein include proteins which are modified specifically to alter a feature of the protein unrelated to its desired physiological activity. The skilled artisan will also realize that conservative amino acid substitutions may be made in the protein to provide functional variants of the foregoing proteins, i.e., the variants which have the functional capabilities of the protein. As used herein, a “conservative amino acid substitution” refers to an amino acid substitution which does not alter the relative charge or size characteristics of the protein in which the amino acid substitution is made. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Such substitutions can be made by a variety of methods known to one of ordinary skill in the art. For example, amino acid substitutions may be made by PCR-directed mutation, site-directed mutagenesis according to the method of Kunkel (Proc. Nat. Acad. Sci. U.S.A. 82: 488-492, 1985), or by chemical synthesis of a gene encoding a protein. Where amino acid substitutions are made to a small unique fragment of a protein the substitutions can be made by directly synthesizing the protein. The activity of functional variants or fragments of protein can be tested by cloning the gene encoding the altered protein into a bacterial or mammalian expression vector, introducing the vector into an appropriate host cell, expressing the altered protein, and testing for a functional capability of the protein as disclosed herein.

Variants of a protein can be prepared according to methods for altering protein sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York.

In general, the treatment methods involve administering a CD43 inhibitor to treat a tumor. In some embodiments, the CD43 inhibitor is a CD43 binding molecule. One example of a CD43 binding molecule is a hnRNP-K molecule.

As used herein, a “hnRNP-K molecule” refers to a hnRNP-K nucleic acid or to a hnRNP-K protein. A hnRNP-K nucleic acid refers to a nucleic acid which encodes a “hnRNP protein” and complements thereof. In some embodiments the hnRNP nucleic acid is selected from the group consisting of:

(a) nucleic acids which hybridize under stringent conditions to a nucleic acid having a nucleotide sequence set forth as SEQ ID NO: 15 (GenBank Accession NO: NM_{—}031263.1) and which code for a hnRNP-K protein,

(b) deletions, additions and substitutions of the nucleic acids of (a),

(c) nucleic acids that differ from the nucleic acids of (a) or (b) in codon sequence due to the degeneracy of the genetic code, and

(d) complements of (a), (b), or (c).

One preferred bnRNP nucleic acid is an hnRNP-K nucleic acid which encodes a hnRNP-K protein having the amino acid sequence of SEQ ID NO: 16 (GenBank Accession NO: AAB20770.1).

Another example of a CD43 binding molecule is a Purα molecule. It has been discovered that the combination of an hnRNP molecule and a Purα molecule produce an even greater physiological response than either molecule alone. As used herein, a “Purα molecule” refers to a Purα nucleic acid or to a Purα protein. A Purα nucleic acid refers to a nucleic acid which encodes a Purα protein. This includes, for instance, nucleic acids selected from the group consisting of:

(a) nucleic acids which hybridize under stringent conditions to a nucleic acid having a nucleotide sequence set forth as SEQ ID NO: 17 (GenBank Accession NO: M96684. 1) and which code for a Purα protein,

(b) deletions, additions and substitutions of the nucleic acids of (a),

(c) nucleic acids that differ from the nucleic acids of (a) or (b) in codon sequence due to the degeneracy of the genetic code, and

(d) complements of (a), (b), or (c).

One preferred Purα nucleic acid is one which encodes a Purα protein having an amino acid sequence of SEQ ID NO: 18 (GenBank Accession NO: NM_{—}005859.2).

The nucleic acids of the invention may be DNA, RNA, mixed deoxyribonucleotides and ribonucleotides, or may also incorporate synthetic non-natural nucleotides.

The term “stringent conditions” as used herein refers to parameters with which the art is familiar. Nucleic acid hybridization parameters may be found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. More specifically, stringent conditions, as used herein, refers, for example, to hybridization at 65° C. in hybridization buffer (3.5×SSC, 0.02% Ficoll, 0.02% polyvinyl pyrrolidone, 0.02% Bovine Serum Albumin, 2.5 mM NaH2PO4 (pH 7), 0.5% SDS, 2 mM EDTA). SSC is 0.15M sodium chloride/0.15M sodium citrate, pH 7; SDS is sodium dodecyl sulphate; and EDTA is ethylenediaminetetracetic acid. After hybridization, the membrane upon which the DNA is transferred is washed at 2×SSC at room temperature and then at 0.1×SSC/0.1×SDS at temperatures up to 68° C.

The foregoing set of hybridization conditions is but one example of stringent hybridization conditions known to one of ordinary skill in the art. There are other conditions, reagents, and so forth which can be used, which result in a stringent hybridization. The skilled artisan will be familiar with such conditions, and thus they are not given here. It will be understood, however, that the skilled artisan will be able to manipulate the conditions in a manner to permit the clear identification of homologs and alleles of the nucleic acids of the invention. The skilled artisan also is familiar with the methodology for screening cells and libraries for expression of such molecules which then are routinely isolated, followed by isolation of the pertinent nucleic acid and sequencing.

In general CD43 homologs and alleles typically will share at least 40% nucleotide identity to SEQ ID NO: 1 (GenBank Accession NO: NM_{—}003123), SEQ ID NO: 3 (GenBank Accession NO: BC012350), SEQ ID NO: 5 (GenBank Accession NO: X60502), SEQ ID NO: 7 (GenBank Accession NO: J04168), SEQ ID NO: 9 (GenBank Accession NO: M61827), SEQ ID NO: 11 (GenBank Accession NO: J04536), or SEQ ID NO: 13 (GenBank Accession NO: X52075) and/or at least 50% amino acid identity to NO: 2, SEQ ID NO: 4, SEQ ID NO: 6, SEQ ID NO: 8, SEQ ID NO: 10, SEQ ID NO: 12, or SEQ ID NO: 14, in some instances will share at least 50% nucleotide identity and/or at least 65% amino acid identity and in still other instances will share at least 60% nucleotide identity and/or at least 75% amino acid identity. Preferred homologs and alleles share nucleotide and amino acid identities with SEQ ID NO: 1 (GenBank Accession NO: NM_{—}003123), SEQ ID NO: 3 (GenBank Accession NO: BC012350), SEQ ID NO: 5 (GenBank Accession NO: X60502), SEQ ID NO: 7 (GenBank Accession NO: J04168), SEQ ID NO: 9 (GenBank Accession NO: M61827), SEQ ID NO: 1, (GenBank Accession NO: J04536), or SEQ ID NO: 13 (GenBank Accession NO: X52075) and SEQ ID NO: 2, SEQ ID NO: 4, SEQ ID NO: 6, SEQ ID NO: 8, SEQ ID NO: 10, SEQ ID NO: 12, or SEQ ID NO: 14, respectively, and encode polypeptides of greater than 80%, more preferably greater than 90%, still more preferably greater than 95% and most preferably greater than 99% identity.

In general, hnRNP-K homologs and alleles typically will share at least 40% nucleotide identity and/or at least 50% amino acid identity to SEQ ID NO: 15 and SEQ ID NO: 16, respectively, in some instances will share at least 50% nucleotide identity and/or at least 65% amino acid identity and in still other instances will share at least 60% nucleotide identity and/or at least 75% amino acid identity. Preferred homologs and alleles share nucleotide and amino acid identities with SEQ ID NO: 15 and SEQ ID NO: 16, respectively, and encode polypeptides of greater than 80%, more preferably greater than 90%, still more preferably greater than 95% and most preferably greater than 99% identity.

Purα homologs and alleles typically will share at least 40% nucleotide identity and/or at least 50% amino acid identity to SEQ ID NO: 17 and SEQ ID NO: 18, respectively, in some instances will share at least 50% nucleotide identity and/or at least 65% amino acid identity and in still other instances will share at least 60% nucleotide identity and/or at least 75% amino acid identity. Preferred homologs and alleles share nucleotide and amino acid identities with SEQ ID NO: 17 and SEQ ID NO: 18, respectively, and encode polypeptides of greater than 80%, more preferably greater than 90%, still more preferably greater than 95% and most preferably greater than 99% identity.

The percent identity can be calculated using various, publicly available software tools developed by NCBI (Bethesda, Md.) that can be obtained through the internet. Exemplary tools include the BLAST system which uses algorithms developed by Altschul et al. (Nucleic Acids Res. 25:3389-3402, 1997). Pairwise and ClustalW alignments (BLOSUM30 matrix setting) as well as Kyte-Doolittle hydropathic analysis can be obtained using the MacVector sequence analysis software (Oxford Molecular Group). Watson-Crick complements of the foregoing nucleic acid molecules also are embraced by the invention.

The invention also includes degenerate nucleic acids which include alternative codons to those present in the native materials. For example, serine residues are encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into an elongating protein such as a CD43 protein, a hnRNP-K protein, or a Purα protein. Similarly, nucleotide sequence triplets which encode other amino acid residues include, but are not limited to: CCA, CCC, CCG and CCT (proline codons); CGA, CGC, CGG, CGT, AGA and AGG (arginine codons); ACA, ACC, ACG and ACT (threonine codons); AAC and AAT (asparagine codons); and ATA, ATC and ATT (isoleucine codons). Other amino acid residues may be encoded similarly by multiple nucleotide sequences. Thus, the invention embraces degenerate nucleic acids that differ from the biologically isolated nucleic acids in codon sequence due to the degeneracy of the genetic code.

The nucleic acids useful herein may be isolated nucleic acids. As used herein, the term “isolated nucleic” acid means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. An isolated nucleic acid as used herein encompasses a non-naturally occurring chromosome as well as total genomic DNA isolated from cells and tissues.

In some aspects of the invention, the treatment methods involve administering to a subject a nucleic acid (e.g. hnRNP-K nucleic acid and/or a Purα nucleic acid) to treat a tumor. In some embodiments, the nucleic acid is introduced ex vivo into the cell(s) of a subject and the cell(s) are returned to the subject. The nucleic acid is under operable control of regulatory elements which permit expression of the nucleic acid in the cell(s). Numerous transfection and transduction techniques as well as appropriate expression vectors are well known to those of ordinary skill in the art, some of which are described in PCT application WO95/00654. In vivo introduction of the hnRNP-K nucleic acid or Purα nucleic acid using vectors such as adenovirus, retroviruses, herpes virus, and targeted liposomes is also contemplated according to the invention.

In preferred embodiments, a virus vector for delivering a nucleic acid encoding a protein of the invention (e.g. hnRNP-protein or Purα protein) is selected from the group consisting of adenoviruses, adeno-associated viruses, poxviruses including vaccinia viruses and attenuated poxviruses, Semliki Forest virus, Venezuelan equine encephalitis virus, retroviruses, Sindbis virus, and Ty virus-like particle. Examples of viruses and virus-like particles which have been used to deliver exogenous nucleic acids include: replication-defective adenoviruses (e.g,, Xiang et al., Virology 219:220-227, 1996; Eloit et al., J. Virol. 7:5375-5381, 1997; Chengalvala et al., Vaccine 15:335-339, 1997), a modified retrovirus (Townsend et al., J. Virol. 71:3365-3374, 1997), a nonreplicating retrovirus (Irwin et al., J. Virol 68:5036-5044, 1994), a replication defective Semliki Forest virus (Zhao et al., Proc. Natl. Acad. Sci. USA 92:3009-3013, 1995), canarypox virus and highly attenuated vaccinia virus derivative (Paoletti, Proc. Natl. Acad. Sci. USA 93:11349-11353, 1996), non-replicative vaccinia virus (Moss, Proc. Natl. Acad. Sci. USA 93:11341-11348, 1996), replicative vaccinia virus (Moss, Dev. Biol. Stand. 82:55-63, 1994), Venzuelan equine encephalitis virus (Davis et al., J. Virol. 70:3781-3787, 1996), Sindbis virus (Pugachev et al., Virology 212:587-594, 1995), and Ty virus-like particle (Allsopp et al., Eur. J. Immunol 26:1951-1959, 1996). In on of the preferred embodiments, the virus vector is an adenovirus.

Another one of the preferred viruses for certain applications is the adeno-associated virus, a double-stranded DNA virus. The adeno-associated virus is capable of infecting a wide range of cell types and species and can be engineered to be replication-deficient. It further has advantages, such as heat and lipid solvent stability, high transduction frequencies in cells of diverse lineages, including hematopoietic cells, and lack of superinfection inhibition thus allowing multiple series of transductions. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion.

In general, other preferred viral vectors are based on non-cytopathic eukcaryotic viruses in which non-essential nucleic acids have been replaced with the nucleic acids of interest. Non-cytopathic viruses include retroviruses, the life cycle of which involves reverse transcription of genomic viral RNA into DNA with subsequent proviral integration into host cellular DNA. In general, the retroviruses are replication-deficient (i.e., capable of directing synthesis of the desired proteins, but incapable of manufacturing an infectious particle). Such genetically altered retroviral expression vectors have general utility for the high-efficiency transduction of nucleic acids in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, M., Gene Transfer and Expression, A Laboratory Manual, W.H. Freeman Co., New York (1990) and Murry, E. J. Ed. Methods in Molecular Biology, vol. 7, Humana Press, Inc., Cliffton, N.J. (1991).

In addition to the viral vectors described above, various techniques may be employed for introducing nucleic acid molecules of the invention into cells, depending on whether the nucleic acid molecules are introduced in vitro or in vivo in a host. Such techniques include transfection of nucleic acid molecule-CaPO₄ precipitates, transfection of nucleic acid molecules associated with DEAE, transfection or infection with the foregoing viruses including the nucleic acid molecule of interest, liposome mediated transfection, and the like. For certain uses, it is preferred to target the nucleic acid molecule to particular cells. In such instances, a vehicle used for delivering a nucleic acid molecule of the invention into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the nucleic acid molecule delivery vehicle. In some embodiments, monoclonal antibodies are preferred. Where liposomes are employed to deliver the nucleic acid molecules of the invention, proteins which bind to a surface membrane protein associated with endocytosis may be incorporated into the liposome formulation for targeting and/or to facilitate uptake. Such proteins include capsid proteins or fragments thereof tropic for a particular cell type, antibodies for proteins which undergo internalization in cycling, proteins that target intracellular localization and enhance intracellular half life, and the like. Polymeric delivery systems also have been used successfully to deliver nucleic acid molecules into cells, as is known by those skilled in the art. Such systems even permit oral delivery of nucleic acid molecules.

In some other aspects of the invention the treatment methods involve administering to a subject a hnRNP-K protein. The term protein, as used herein encompasses the terms polypeptide and peptide. As used herein a “hnRNP-K protein” refers to a protein having the activity of a native hnRNP-K protein such as that described by SEQ ID NO: 16 as well as functionally active fragments or variants thereof. Thus, for example a hnRNP-K protein includes proteins which include the proteins of SEQ ID NO: 16, unique fragments, and functional variants thereof, as well as proteins which are encoded by nucleic acids such as those having SEQ ID NO: 15, provided that the functional fragment or variant exhibits a hnRNP-K functional activity. As used herein, a hnRNP-K functional activity refers to the ability of a hnRNP-K protein to modulate one or more of the following parameters such as: suppressing tumor growth and/or cell proliferation, reducing the number of white blood cells, and repressing CD molecule expression. An exemplary hnRNP-K functional activity is a tumor suppressor activity such as suppressing and/or reducing tumor cell growth, proliferation, and/or metastasis.

A hnRNP-K functional activity can be determined, for example, by assaying tumor size in a biological sample. An exemplary assay involves: measuring the tumor size in a biological sample before treatment with a putative hnRNP-K molecule, measuring the tumor size in the biological sample after treatment with the putative hnRNP-K molecule, and comparing the tumor size in the biological sample before and after treatment with the putative hnRNP-K molecule. In this example, a decrease or even less of an increase than would be observed in the absence of such treatment, in the tumor size in the biological sainple after treatment with the putative hnRNP-K molecule indicates that the putative hnRNP-K molecule is an hnRNP-K having a hnRNP-K functional activity. An increase in the tumor size in the biological sample after treatment with the putative hnRNP-K molecule over that which would occur in the absence of a hnRNP-K molecule indicates that the putative hnRNP-K molecule is not a hnRNP molecule of the invention and does not have a hnRNP-K functional activity.

The hnRNP-K functional activity can be determined ex vivo or in vitro. An ex vivo or in vitro hnRNP-K functional activity assay involves, for example, assaying the number of tumor cells in an ex vivo medium or in an in vitro sample. The assay involves: measuring the number of tumor cells in an ex vivo medium or in vitro sample before treatment with a putative hnRNP-K molecule, measuring the number of tumor cells in the ex vivo medium or in vitro sample after treatment with the putative hnRNP-K molecule, and comparing the number of tumor cells in the ex vivo medium or in vitro sample before and after treatment with the putative hnRNP-K molecule. A decrease or less of an increase (compared to a control) in the number of tumor cells in the ex vivo medium or in vitro sample after treatment with the putative hnRNP-K molecule indicates that the putative hnRNP-K molecule has a hnRNP-K functional activity. A suitable control is a known hnRNP-K protein such as that identified by SEQ ID NO: 16. An increase in the number of tumor cells in comparison with a control in the ex vivo medium or in vitro sample after treatment with the putative hnRNP-K molecule indicates that the putative hnRNP-K molecule is not a hnRNP-K molecule of the invention and does not have a hnRNP-K functional activity.

Although not wishing to be bound to any particular theory or mechanism, it is believed that the hnRNP-K protein may affect at least some of the above-noted cell functions by inhibiting a CD promoter. Thus in vitro assays analyzing CD promoter activation with putative hnRNP-K molecules may also be useful for identifying hnRNP-K molecules having hnRNP-K functional activity.

The treatment methods may optionally involve administering to a subject a Purα protein. As used herein a Purα protein refers to a protein having the activity of a native Purα protein such as that described by SEQ ID NO: 18 as well as functionally active fragments or variants thereof. Thus, for example a Purα protein includes proteins which include the proteins of SEQ ID NO: 18, unique fragments, and functional variants thereof, as well as proteins which are encoded by nucleic acids such as those having SEQ ID NO: 17, provided that the functional fragment or variant exhibits a Purα functional activity. As used herein, a Purα functional activity refers to the ability of a Purα protein to modulate one or more of the following parameters such as: suppressing tumor growth and/or cell proliferation, reducing the number of white blood cells, and repressing CD molecule expression. An exemplary Purα functional activity is a tumor suppressor activity such as suppressing and/or reducing tumor cell growth, proliferation, and/or metastasis. A Purα functional activity can be determined using any of the exemplary assays set forth above for hnRNP-K molecules.

The hnRNP-K proteins are isolated proteins. Proteins can be isolated from biological samples including tissue or cell homogenates, and can also be expressed recombinantly in a variety of prokaryotic and eukaryotic expression systems by constructing an expression vector appropriate to the expression system, introducing the expression vector into the expression system, and isolating the recombinantly expressed protein. Short proteins, also can be synthesized chemically using well-established methods of peptide synthesis.

Thus, as used herein with respect to proteins, “isolated” means separated from its native environment and present in sufficient quantity to permit its identification or use. Isolated, when referring to a protein means, for example: (i) selectively produced by expression of a recombinant nucleic acid or (ii) purified as by chromatography or electrophoresis. Isolated proteins may, but need not be, substantially pure. The term “substantially pure” means that the proteins are essentially free of other substances with which they may be found in nature or in vivo systems to an extent practical and appropriate for their intended use. Substantially pure proteins may be produced by teclniques well known in the art. Because an isolated protein may be admixed with a pharmaceutically acceptable carrier in a pharmaceutical preparation, the protein may comprise only a small percentage by weight of the preparation. The protein is nonetheless isolated in that it has been separated from the substances with which it may be associated in living systems, e.g. isolated from other proteins.

A fragment of a protein (CD43, hnRNP-K, or Purα for example) generally has the features and characteristics of fragments including unique fragments. As will be recognized by those skilled in the art, the size of a fragment which is unique will depend upon factors such as whether the fragment constitutes a portion of a conserved protein domain. Thus, some regions of proteins will require longer segments to be unique while others will require only short segments, typically between 5 and 12 amino acids (e.g. 5, 6, 7, 8, 9, 10, 11, and 12 amino acids long).

Unique fragments of a protein preferably are those fragments which retain a distinct functional capability of the protein. Functional capabilities which can be retained in a fragment of a protein include interaction with antibodies, interaction with other proteins or fragments thereof, selective binding of nucleic acid molecules, and enzymatic activity. One important activity is the ability to act as a signature for identifying the protein.

Those skilled in the art are well versed in methods for selecting unique amino acid sequences, typically on the basis of the ability of the fragment to selectively distinguish the sequence of interest from non-family members. A comparison of the sequence of the fragment to those on known data bases typically is all that is necessary.

The invention embraces variants of the hnRNP-K proteins described herein. As used herein, a “variant” of a hnRNP-K protein is a protein which contains one or more modifications to the primary amino acid sequence of a hnRNP-K protein. Modifications which create a hnRNP-K protein variant can be made to a hnRNP-K protein 1) to produce, increase, reduce, or eliminate activity of the hnRNP-K protein; 2) to enhance a property of the hnRNP-K protein, such as protein stability in an expression system or the stability of protein-protein binding; 3) to provide a novel activity or property to a hnRNP-K protein; or 4) to provide equivalent or better binding to an CD promoter. Modifications to a hnRNP-K protein are typically made to the hnRNP-K nucleic acid which encodes the hnRNP-K protein, and can include deletions, point mutations, truncations, amino acid substitutions and additions of amino acids or non-amino acid moieties. Alternatively, modifications can be made directly to the protein, such as by cleavage, addition of a linker molecule, addition of a detectable moiety, such as biotin, addition of a fatty acid, and the like. Modifications also embrace fusion proteins comprising all or part of the hnRNP-K amino acid sequences.

The invention also embraces variants of the Purα proteins described herein. As used herein, a “variant” of a Purα protein is a protein which contains one or more modifications to the primary amino acid sequence of a Purα protein. Modifications which create a Purα protein variant can be made to a Purα protein 1) to produce, increase, reduce, or eliminate activity of the Purα protein; 2) to enhance a property of the Purα protein, such as protein stability in an expression system or the stability of protein-protein binding; 3) to provide a novel activity or property to a Purα protein; or 4) to provide equivalent or better binding to an CD promoter. Modifications to a Purα protein are typically made to the Purα nucleic acid which encodes the Purα protein, and can include deletions, point mutations, truncations, amino acid substitutions and additions of amino acids or non-amino acid moieties. Alternatively, modifications can be made directly to the protein, such as by cleavage, addition of a linker molecule, addition of a detectable moiety, such as biotin, addition of a fatty acid, and the like. Modifications also embrace fusion proteins comprising all or part of the Purα amino acid sequences.

Upon determining that a peptide derived from a hnRNP-K protein plays a role in, for example, suppression of tumor growth and/or cell proliferation, reduction in the number of white blood cells, or repression of CD molecule expression, one can make conservative amino acid substitutions to the amino acid sequence of the peptide to make a variant of a hnRNP-K protein. The substituted peptides can then be tested for one or more of the above-noted functions, in vivo or in vitro. These variants can be tested for improved stability and are useful, inter alia, in pharmaceutical compositions.

Exemplary functional variants of the hnRNP-K protein include conservative amino acid substitutions of proteins encoded by SEQ ID NO: 16. Conservative amino-acid substitutions in the amino acid sequence of the hnRNP-K protein to produce functional variants of hnRNP-K protein typically are made by alteration of the nucleic acid molecule encoding a hnRNP-K protein. Exemplary functional variants of the Purα protein include conservative amino acid substitutions of proteins encoded by SEQ ID NO: 18. Conservative amino-acid substitutions in the amino acid sequence of the Purα protein to produce functional variants of a Purα protein typically are made by alteration of the nucleic acid molecule encoding a Purα protein.

The invention also embraces CD43 antisense molecules that selectively bind to a nucleic acid molecule encoding a CD43 nucleic acid, unique fragments, or complements of these nucleic acid molecules. A unique fragment is one that is a ‘signature’ for the larger nucleic acid. It, for example, is long enough to assure that its precise sequence is not found in molecules outside of the defined nucleic acid molecules. Those of ordinary skill in the art may apply no more than routine procedures to determine if a fragment is unique within the genome.

As used herein, the term “antisense oligonucleotide” or “antisense” describes an oligonucleotide that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridization with the target gene or transcript. Those skilled in the art will recognize that the exact length of the antisense oligonucleotide and its degree of complementarity with its target will depend upon the specific target selected, including the sequence of the target and the particular bases which comprise that sequence.

It is preferred that the antisense oligonucleotide be constructed and arranged so as to bind selectively with the target under physiological conditions, i.e., to hybridize substantially more to the target sequence than to any other sequence in the target cell under physiological conditions. Based upon CD43 antisense molecules, the known sequences for CD43 or upon allelic or homologous genomic and/or cDNA sequences, one of skill in the art can easily choose and synthesize any of a number of appropriate antisense molecules for use in accordance with the present invention. In order to be sufficiently selective and potent for inhibition, such antisense oligonucleotides should comprise at least 10 and, more preferably, at least 15 consecutive bases which are -complementary to the target, although in certain cases modified oligonucleotides as short as 7 bases in length have been used successfully as antisense oligonucleotides (Wagner et al., Nat. Med. 1(11): 1116-1118, 1995). Even more preferably, the antisense oligonucleotides comprise a complementary sequence of 20-35 bases. Although oligonucleotides may be chosen which are antisense to any region of the gene (such as to the CD43) or mRNA transcripts, the antisense oligonucleotides preferably correspond to N-terminal or 5′ upstream sites such as translation initiation, transcription initiation or promoter sites. In addition, 3′-untranslated regions may be targeted by antisense oligonucleotides. Targeting to mRNA splicing sites has also been used in the art but may be less preferred if alternative mRNA splicing occurs. In addition, the antisense is targeted, preferably, to sites in which mRNA secondary structure is not expected (see, e.g., Sainio et al., Cell Mol. Neurobiol. 14(5):439-457, 1994) and at which proteins are not expected to bind.

In some embodiments, the antisense oligonucleotides of the invention may be composed of “natural” deoxyribonucleotides, ribonucleotides, or any combination thereof. That is, the 5′ end of one native nucleotide and the 3′ end of another native nucleotide may be covalently linked, as in natural systems, via a phosphodiester internucleoside linkage. These oligonucleotides may be prepared by art recognized methods which may be carried out manually or by an automated synthesizer. They also may be produced recombinantly by vectors.

In some embodiments, however, the antisense oligonucleotides of the invention also may include “modified” oligonucleotides. That is, the oligonucleotides may be modified in a number of ways which do not prevent them from hybridizing to their target but which enhance their stability or targeting or which otherwise enhance their therapeutic effectiveness.

The term “modified oligonucleotide” as used herein describes an oligonucleotide in which (1) at least two of its nucleotides are covalently linked via a synthetic internucleoside linkage (i.e., a linkage other than a phosphodiester linkage between the 5′ end of one nucleotide and the 3′ end of another nucleotide) and/or (2) a chemical group not normally associated with nucleic acids has been covalently attached to the oligonucleotide. Preferred synthetic internucleoside linkages are phosphorothioates, alkylphosphonates, phosphorodithioates, phosphate esters, alkylphosphonothioates, phosphoramidates, carbamates, carbonates, phosphate triesters, acetamidates, carboxymethyl esters and peptides.

The term “modified oligonucleotide” also encompasses oligonucleotides with a covalently modified base and/or sugar. For example, modified oligonucleotides include oligonucleotides having backbone sugars which are covalently attached to low molecular weight organic groups other than a hydroxyl group at the 3′ position and other than a phosphate group at the 5′ position. Thus modified oligonucleotides may include a 2′-O-alkylated ribose group. In addition, modified oligonucleotides may include sugars such as arabinose instead of ribose.

Delivery systems for molecules of the invention can include time-release, delayed release or sustained release delivery systems. Such systems can avoid repeated administrations of the CD43 inhibitor increasing convenience to the subject and the physician. Many types of release delivery systems are available and known to those of ordinary skill in the art. They include polymer base systems such as poly(lactideglycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

Use of a long-term sustained release implant may be particularly suitable for treatment of chronic conditions. Long-term release, as used herein, means that the implant is constructed and arranged to deliver therapeutic levels of the active ingredient for at least 30 days, and preferably 60 days. Long-term sustained release implants are well-known to those of ordinary skill in the art and include some of the release systems described above.

As used herein, a subject is a mammal such as a human, non-human primate, cow, horse, pig, sheep, goat, dog, cat, or rodent. In some preferred embodiments, the subject is a human. Exemplary subjects calling for treatment with a CD43 inhibitor include subjects having or at risk of having a tumor (solid and non-solid). In some embodiments a subject is free of disorders previously known to be treated using a CD43 inhibitor.

A subject having a tumor is a subject with at least one identifiable sign, symptom, or laboratory finding sufficient to make a diagnosis of a tumor in accordance with clinical standards known in the art for identifying such tumors. Examples of such clinical standards can be found in Harrison's Principles of Internal Medicine, 15th Ed., Fauci A S et al., eds., McGraw-Hill, New York, 2001. In some instances, a diagnosis of a tumor will include identification of a particular cell type present in a sample of a body fluid or tissue obtained from the subject.

A subject at risk of having a tumor is a subject with an identifiable risk factor for having a tumor. For example, a subject at risk of having a tumor can include an individual with a known or suspected exposure to environmental agents (e.g., carcinogens) associated with an increased risk of having a tumor. Additionally or alternatively, a subject at risk of having a tumor can include an individual with a genetic predisposition to developing a tumor. Yet other examples of a subject at risk of having a tumor include a subject that previously has been diagnosed with a cancer associated with a tumor and who is at risk of metastasis of the primary tumor.

As used herein, “treating” includes preventing, delaying, abating or arresting the clinical symptoms and or signs of a tumor. Treatment also includes reducing or preventing tumor cell growth, proliferation, and/or metastasis as well as increasing the resistance of a subject to develop a disease.

Tumors encompassed by the invention include, for example, benign and malignant solid tumors and benign and malignant non-solid tumors. Examples of solid tumors include but are not limited to: biliary tract cancer, brain cancer (including glioblastomas and medulloblastomas), breast cancer, cervical cancer, choriocarcinoma, colon cancer, endometrial cancer, esophageal cancer, gastric cancer, intraepithelial neoplasms, including Bowen's disease and Paget's disease, liver cancer, lung cancer, lymphomas, including Hodgkin's disease and lymphocytic lymphomas, neuroblastomas, oral cancer, including squamous cell carcinoma, ovarian cancer, including those arising from epithelial cells, stromal cells, germ cells and mesenchymal cells, pancreatic cancer, prostate cancer, rectal cancer, renal cancer including adenocarcinoma and Wilms tumor, sarcomas, including leiomyosarcoma, rhabdomyosarcoma, liposarcoma, fibrosarcoma and osteosarcoma, skin cancer, including melanoma, Kaposi's sarcoma, basocellular cancer and squamous cell cancer, testicular cancer, including germinal tumors (seminomas, and non-seminomas such as teratomas and choriocarcinomas), stromal tumors and germ cell tumors, and thyroid cancer, including thyroid adenocarcinoma and medullary carcinoma.

Examples of non-solid tumors include but are not limited to hematological neoplasms. As used herein, a hematologic neoplasm is a term of art which includes lymphoid disorders, myeloid disorders, and AIDS associated leukemias.

Lymphoid disorders include but are not limited to acute lymphocytic leukemia and chronic lymphoproliferative disorders (e.g., lymphomas, myelomas, and chronic lymphoid leukemias). Lymphomas include, for example, Hodgkin's disease and non-Hodgkin's lymphoma. Chronic lymphoid leukemias include, for example, T cell chronic lymphoid leukemias and B cell chronic lymphoid leukemias.

Myeloid disorders include chronic myeloid disorders such as for instance chronic myeloproliferative disorders and myelodysplastic syndrome and acute myeloid leukemia. Chronic myeloproliferative disorders include but are not limited to angiogenic myeloid metaplasia, essential thrombocythemia, chronic myelogenous leukemia, polycythemia vera, and atypical myeloproliferative disorders. Atypical myeloproliferative disorders include atypical chronic myelogenous leukemia, chronic neutrophilic leukemia, mast cell disease, and chronic eosinophilic leulemia.

The CD43 inhibitor may be administered alone or in conjunction with one or more therapies known or believed to be useful for treating a tumor. Such therapies include, for example, anti-tumor therapy such as radiation therapy, surgery, or a chemotherapeutic agent or a combination thereof. As used herein, an anti-tumor therapy refers to a therapy that is administered to a subject for the purpose of treating a tumor. Various types of therapies for the treatment of tumors are described herein. Anti-tumor therapies include immunotherapeutic agents, tumor vaccines, hormone therapy, and biological response modifiers.

Anti-tumor therapies function in a variety of ways. For example, some anti-tumor therapies work by targeting physiological mechanisms that are specific to tumor cells. Examples include the targeting of specific genes and their gene products (i.e., proteins primarily) which are mutated in tumors. Such genes include but are not limited to oncogenes (e.g., Ras, Her2, bcl-2), tumor suppressor genes (e.g., EGF, p53, Rb), and cell cycle targets (e.g., CDK4, p21, telomerase). Some anti-tumor therapies can alternately target signal transduction pathways and molecular mechanisms which are altered in tumor cells. Targeting of tumor cells via the epitopes expressed on their cell surface is accomplished through the use of monoclonal antibodies. This latter type of anti-tumor therapy is generally referred to herein as immunotherapy.

Other anti-tumor therapies target cells other than tumor cells. For example, some anti-tumor therapies prime the immune system to attack tumor cells (i.e., tumor vaccines). Still other anti-tumor therapies, called angiogenesis inhibitors, function by attacking the blood supply of solid tumors. Since the most malignant tumors are able to metastasize (i.e., exist the primary tumor site and seed a distal tissue, thereby forming a secondary tumor), therapies that impede this metastasis are also useful in the treatment of tumor. Angiogenic mediators include basic FGF, VEGF, angiopoietins, angiostatin, endostatin, TNF-α, TNP-470, thrombospondin-1, platelet factor 4, CAI, and certain members of the integrin family of proteins. One category of this type of anti-tumor therapy is a metalloproteinase inhibitor, which inhibits the enzymes used by the tumor cells to exit the primary tumor site and extravasate into another tissue.

Immunotherapeutic agents are medicaments which derive from antibodies or antibody fragments which specifically bind or recognize a tumor antigen. As used herein a tumor antigen is broadly defined as an antigen expressed by a tumor cell. In some embodiments, the antigen is expressed at the cell surface of the tumor cell. In some preferred embodiments, the antigen is one which is not expressed by normal cells, or at least not expressed to the same level as in tumor cells. Antibody-based immunotherapies may function by binding to the cell surface of a tumor cell and thereby stimulate the endogenous immune system to attack the tumor cell. Another way in which antibody-based therapy functions is as a delivery system for the specific targeting of toxic substances to tumor cells. Antibodies are usually conjugated to toxins such as ricih (e.g., from castor beans), calicheamicin and maytansinoids, to radioactive isotopes such as Iodine-131 and Yttrium-90, to chemotherapeutic agents (as described herein), or to biological response modifiers. In this way, the toxic substances can be concentrated in the region of the tumor and non-specific toxicity to normal cells can be minimized. In addition to the use of antibodies which are specific for tumor antigens, antibodies which bind to vasculature, such as those which bind to endothelial cells, are also useful in the invention. This is because generally solid tumors are dependent upon newly formed blood vessels to survive, and thus most tumors are capable of recruiting and stimulating the growth of new blood vessels. As a result, one strategy of many tumor medicaments is to attack the blood vessels feeding a tumor and/or the connective tissues (or stroma) supporting such blood vessels.

Chemotherapeutic agents which can be used according to the invention include but are not limited to Aminoglutethimide, Asparaginase, Busulfan, Carboplatin, Chlorombucil, Cytarabine HCl, Dactinomycin, Daunorubicin HCl, Estramustine phosphate sodium, Etoposide (VP16-213), Floxuridine, Fluorouracil (5-FU), Flutamide, Hydroxyurea (hydroxycarbamide), Ifosfamide, Interferon Alfa-2a, Alfa-2b, Leuprolide acetate (LHRH-releasing factor analogue), Lomustine (CCNU), Mechlorethamine HCl (nitrogen mustard), Mercaptopurine, Mesna, Mitotane (o.p-DDD), Mitoxantrone HCl, Octreotide, Plicamycin, Procarbazine HCl, Streptozocin, Tamoxifen citrate, Thioguanine, Thiotepa, Vinblastine sulfate, Amsacrine (m-AMSA), Azacitidine, Erythropoietin, Hexamethylmelamine (HMM), Interleukin 2, Mitoguazone (methyl-GAG; methyl glyoxal bis-guanylhydrazone; MGBG), Pentostatin (2′deoxycoformycin), Semustine (methyl-CCNU), Teniposide (VM-26) and Vindesine sulfate.

Tumor vaccines are medicaments which are intended to stimulate an endogenous immune response against tumor cells. Currently produced vaccines predominantly activate the humoral immune system (i.e., the antibody dependent immune response). Other vaccines currently in development are focused on activating the cell-mediated immune system including cytotoxic T lymphocytes which are capable of killing tumor cells. Tumor vaccines generally enhance the presentation of tumor antigens to both antigen presenting cells (e.g., macrophages and dendritic cells) and/or to other immune cells such as T cells, B cells, and NK cells. In some instances, tumor vaccines may be used along with adjuvants, such as those described above.

Tumor antigens, such as those present in tumor vaccines or those used to prepare tumor immunotherapies, can be prepared from crude tumor cell extracts, as described in Cohen P A et al. (1994) Cancer Res 54:1055-8, or by partially purifying the antigens, using recombinant technology, or de novo synthesis of known antigens. Tumor antigens can be used in the form of immunogenic portions of a particular antigen or in some instances a whole cell or a tumor mass can be used as the antigen. Such antigens can be isolated or prepared recombinantly or by any other means known in the art.

Other vaccines take the form of dendritic cells which have been exposed to tumor antigens in vitro, have processed the antigens and are able to express the tumor antigens at their cell'surface in the context of MHC molecules for effective antigen presentation to other immune system cells. Dendritic cells form the link between the innate and the acquired immune system by presenting antigens and through their expression of pattern recognition receptors which detect microbial molecules like LPS in their local environment.

The invention also provides a method of reducing the number of white blood cells in a subject. The method involves administering to a subject in need of such reduction in the number of white blood cells, a CD43 inhibitor in an effective amount to reduce the number of white blood cells in said subject. Exemplary subjects in need of a reduction of the number of white blood cells include subjects with white blood cell disorders such as acute leukemias, chronic leukemias, and lymphomas. Other examples of subjects requiring reduction in the number of white blood cells include subjects who are about to undergo, are undergoing, or have undergone a bone marrow, organ, cellular, or material transplant. A subject about to undergo a bone marrow, organ, cellular, or material transplant is a subject having a disorder requiring a bone marrow, organ, cellular or material transplant.

As used herein reduction of the number of white bloods refers to decreasing the number of existing white blood cells or decreasing the production of new white blood cells. Decreasing, as used herein, includes total elimination or eradication, as well as other decreases which do not result in total eradication.

A transplant is an organ, tissue, a cell(s), or a material for grafting into an area of the body or into another individual: A transplant encompasses an organ, tissue, or a cell(s) taken from the body for grafting into another area of the same body or into another individual. A transplant also encompasses ex-vivo material (organ, tissue, cell(s) or material inert or non-inert, artificial or non-artificial) for grafting into an area of the body. As used herein, ex-vivo material refers to material prepared outside the body. Examples of transplants include: allogeneic transplants (transplants of tissue between genetically dissimilar animals of the same species, heterotopic transplants (transplants of tissue typical of one area to a different recipient site), orthotopic or homotypic transplants (transplants of tissue typical of one area to an identical recipient site), syngeneic transplants (transplants of tissues between animals in the same pure line, e.g., within an inbred strain), syngenesioplastic transplants (transplants of tissue from on individual to a related individual of the same species).

The CD43 inhibitor may be administered alone or in conjunction with one or more therapies known or believed to be useful to reduce the number of white blood cells. Examples of medicaments that reduce the number of white blood cells include for example, cytarabine (cytosine arabinoside, anthracyclines such as daunorubicin, Atra, arsenic trioxide, hydroxyurea, busulphan, homoharringtonine, and interferons (IFN) such as IFN-α, IFN-β, and IFN-γ.

Those skilled in the art will recognize which of the other therapies to be administered in conjunction with the CD43 inhibitor are appropriate for treating a given suspected or identified tumor or reducing the number of white blood cells.

The invention provides pharmaceutical compositions of CD43 inhibitor. The pharmaceutical compositions contains a CD43 inhibitor in a pharmaceutically acceptable carrier. Thus, in a related aspect, the invention provides a method for forming a medicament that involves placing a therapeutically effective amount of the CD43 inhibitor in the pharmaceutically acceptable carrier to form one or more doses.

When administered, the therapeutic compositions of the present invention are administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines and optionally other therapeutic agents.

The term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art.

An effective amount means, with respect to a tumor for example, that amount of a CD43 inhibitor which prevents the onset of, alleviates the symptoms of, or stops or slows the progression of a tumor or reduces the number of white blood cells. In general such symptoms are, at least in part, the result of unwanted cell proliferation cells in vivo. Thus, a tumor is a condition that is characterized by certain clinical features and which, it is generally believed, is associated with unwanted cell proliferation cells in vivo. “Unwanted,” with respect to cell proliferation cells in vivo, refers to cell proliferation which results in an adverse medical condition. The phrase “therapeutically effective amount” means, with respect to treatment of a tumor or reduction in the number of white blood cells which prevents the onset of, alleviates the symptoms of, or stops the signs of a tumor or reduces the number of white blood cells.

The pharmaceutical preparations disclosed herein are prepared in accordance with standard procedures and are administered at dosages that are selected to reduce, prevent or eliminate the condition (See, e.g., Remington's Pharmaceutical Sciences, Mack Publishing Company, Easton, Pa., and Goodman and Gilman's The Pharmaceutical Basis of Therapeutics, Pergamon Press, New York, N.Y., the contents of which are incorporated herein by reference, for a general description of the methods for administering various agents for human therapy).

The pharmaceutically acceptable compositions of the present invention comprise a CD43 inhibitor in association with one or more nontoxic, pharmaceutically acceptable carriers and/or diluents and/or adjuvants and/or excipients, collectively referred to herein as “carrier” materials, and if desired other active ingredients.

The CD43 inhibitor of the present invention may be administered by any route, preferably in the form of a pharmaceutical composition adapted to such a route, and would be dependent on the condition being treated. The compounds and compositions may, for example, be administered orally, intravascularly, intramuscularly, subcutaneously, intraperitoneally, or topically. Preferred routes of administration include oral and intravenous administration.

For oral administration, the CD43 inhibitor may be in the form of, for example, a tablet, capsule, suspension or liquid. The pharmaceutical composition is preferably made in the form of a dosage unit containing a therapeutically effective amount of the active ingredient. Examples of such dosage units are tablets and capsules. For therapeutic purposes, the tablets and capsules can contain, in addition to the active ingredient, conventional carriers such as binding agents, for example, acacia gum, gelatin, polyvinylpyrrolidone, sorbitol, or tragacanth; fillers, for example, calcium phosphate, cellulose, glycine, lactose, maize-starch, mannitol, sorbitol, or sucrose; lubricants, for example, magnesium stearate, polyethylene glycol, silica, or talc; disintegrants, for example potato starch, flavoring or coloring agents, or acceptable wetting agents. Oral liquid preparations generally in the form of aqueous or oily solutions, suspensions, emulsions, syrups or elixirs may contain conventional additives such as suspending agents, emulsifying agents, non-aqueous agents, preservatives, coloring agents and flavoring agents. Examples of additives for liquid preparations include acacia, almond oil, ethyl alcohol, fractionated coconut oil, gelatin, glucose syrup, glycerin, hydrogenated edible fats, lecithin, methyl cellulose, methyl or propyl para-hydroxybenzoate, propylene glycol, sorbitol, or sorbic acid.

The pharmaceutical compositions may also be administered via injection. Formulations for parenteral administration may be in the form of aqueous or non-aqueous isotonic sterile injection solutions or suspensions. These solutions or suspensions may be prepared from sterile powders or granules having one or more of the carriers mentioned for use in the formulations for oral administration. The compounds may be dissolved in polyethylene glycol, propylene glycol, ethanol, corn oil, benzyl alcohol, sodium chloride, sterile water, and/or various buffers.

For topical use the compounds of the present invention may also be prepared in suitable forms to be applied to the skin, or mucus membranes of the nose and throat, and may take the form of creams, ointments, liquid sprays or inhalants, lozenges, or throat paints. Such topical formulations further can include chemical compounds such as dimethylsulfoxide (DMSO) to facilitate surface penetration of the active ingredient. Suitable carriers for topical administration include oil-in-water or water-in-oil emulsions using mineral oils, petrolatum and the like, as well as gels such as hydrogel. Alternative topical formulations include shampoo preparations, oral pastes and mouthwash.

For rectal administration the compounds of the present invention may be administered in the form of suppositories admixed with conventional carriers such as cocoa butter, wax or other glyceride.

Alternatively, the compounds of the present invention may be in powder form for reconstitution at the time of delivery.

The dosage regimen for treating a tumor or for reducing the number of white blood cells with a CD43 inhibitor is selected in accordance with a variety of factors, including the type, age, weight, sex and medical condition of the subject, the severity of the disease, the route and frequency of administration, the renal and hepatic function of the subject, and the particular compound employed. An ordinarily skilled physician or clinician can readily determine and prescribe the effective amount of the drug required to treat a tumor or to reduce the number of white blood cells. In general, dosages are determined in accordance with standard practice for optimizing the correct dosage for treating a tumor, reducing the number of white blood cells, or inhibiting a CD promoter.

The dosage regimen can be determined, for example, by following the response to the treatment in terms clinical signs. Examples of such clinical signs are well known in the art, and they include for example the pulse, blood pressure, temperature, and respiratory rate. Harrison's Principles of Internal Medicine, 15th Ed., Fauci AS et al., eds., McGraw-Hill, New York, 2001.

Typically dosages of the CD43 inhibitor will be dependent upon the condition to be treated. In general, the active agent concentration will range from between 0.01 mg per kg of body weight per day (mg/kg/day) to about 10.0 mg/kg/day. Alternatively, the dosages of the CD43 inhibitor will range from between 0.01 micromole per kg of body weight per day (μmole/kg/day) to about 10 μmole/kg/day. Preferred oral dosages in humans may range from daily total dosages of about 1 -1000 mg/day over the effective treatment period. Preferred intravenous dosages in humans may range from daily total dosages of about 1-100 mg/day over the effective treatment period.

In general, doses of radionuclide delivered by the CD43 antibodies of the invention can range from about 0.01 mCi/Kg to about 10 mCi/kg. Preferably the dose of radionuclide ranges from about 0.1 mCi/Kg to about 1.0 mCi/kg. The optimal dose of a given isotope can be determined empirically by simple routine titration experiments well known to one of ordinary skill in the art.

In yet another aspect, the invention provides a method of inhibiting a CD promoter. The method involves contacting the CD promoter with a CD43 inhibitor in an amount effective to inhibit the CD promoter. CD (cluster of differentiation) refers to a specific antigenic marker found on cells such as lymphocytes. A promoter is a nucleotide sequence of DNA to which RNA polymerase binds and initiates transcription. It usually lies upstream of (5′ to) a coding sequence. A promoter sequence aligns the RNA polymerase so that transcription will initiate at a specific site. CD promoters include but are not limited to: CD43, CD11a, CD11b, CD11c, and CD11d.

As used herein, “inhibiting a CD promoter” refers to decreasing or slowing the activity of the CD promoter including halting or eliminating the activity of the CD promoter temporarily or permanently. Inhibition of a CD promoter by a CD43 inhibitor can be determined, for example, by assaying RNA production or expression of a CD molecule in a sample. Such an assay involves: measuring the expression of the CD molecule in a sample before treatment with a CD43 inhibitor, measuring the expression of the CD molecule in the sample after treatment with a CD43 inhibitor, and comparing the expression of the CD molecule before and after treatment with the CD43 inhibitor. A decrease or no increase in the expression of the CD molecule after treatment with the CD43 inhibitor indicates that CD promoter is inhibited by the CD43 inhibitor. An increase in the expression of the CD molecule after treatment with the CD43 inhibitor indicates the CD promoter is not inhibited by the CD43 inhibitor.

In some embodiments of the invention the CD promoter is inhibited in a biological sample in vivo. In other embodiments the CD promoter is inhibited in a biological sample in vitro. As used herein, inhibition of the CD promoter refers to decreasing the function of the CD promoter including a total elimination of the CD promoter function.

The biological sample having the CD promoter be located in vivo or in vitro. For example, the biological sample can be a tissue in vivo. Alternatively, the biological sample can be located in vitro (e.g., a blood sample, tumor biopsy, tissue extract). In a particularly preferred embodiment, the biological sample can be a cell-containing sample, more preferably a sample containing tumor cells or white blood cells. Samples of tissue and/or cells for use in the various methods described herein can be obtained through standard methods. Samples can be surgical samples of any type of tissue or body fluid. Samples can be used directly or processed to facilitate analysis (e.g., paraffin embedding). Exemplary samples include a cell, a cell scraping, a cell extract, a blood sample, a tissue biopsy, including punch biopsy, a tumor biopsy, a bodily fluid, a tissue, or a tissue extract or other methods.

The invention will be more fully understood by reference to the following examples. These examples, however, are merely intended to illustrate the embodiments of the invention and are not to be construed to limit the scope of the invention.

### EXAMPLES

### Example 1

Introduction

CD43 is an abundant, heavily glycosylated molecule expressed specifically on the surface of leukocytes and platelets. When leukocytes are at rest CD43 acts to prevent both homotypic and heterotypic interactions. However, during leukocyte activation CD43 expression is repressed facilitating the intercellular contact required for chemotaxis, phagocytosis, aggregation, adhesion to endothelium and transendothelial migration. In this way CD43 repression contributes to both innate and acquired immunity. Here we report that a dramatic down-regulation of CD43 MRNA levels occurs during activation of the leukocytic cell line K562. This repression coincides with repression of the transcriptional activity of the CD43 gene promoter.

We investigated the possibility that CD43 repression is mediated by transcriptional events. We have previously reported that transcriptional repression of the CD43 promoter occurs during activation of the monocytic cell line U937 and that this is mediated by the transcription factor Purα (Shelley et al., (2001) Br Journal of Haematology, 115:159-166). Here we report that transcriptional repression of the CD43 promoter also occurs during activation of the pre-erythroid/pre-megakaryocytic cell line K562. We demonstrated that heterogeneous nuclear ribonucleoprotein K (hnRNP-K) alone and in combination with Purα mediates repression of the CD43 promoter during K562 activation. HnRNP-K and Purα bind single-stranded DNA.

Materials and Methods

Cells and Culture Media: Jurkat T-lymphocytic cells and K562 pre-erythroid/pre-megakaryocytic cells were obtained from the American Type Culture Collection (ATCC) and grown in RPMI 1640 medium supplemented with 10% fetal calf serum, 2 mM L-glutamine, 100 U/mI penicillin and 100 μg/,ml streptomycin. K562 cells were activated by the addition of phorbol 12-myristate 13-acetate (PMA) (Sigma Chemical Co., St Louis, Mo.) to a final, concentration of 100 ng/ml. K562 cells stably transfected with the −2/+99 CD43 promoter linked to the luciferase reporter gene were selected using 300 μg/ml of zeocin (Invitrogen, Carlsbad, Calif.). The DNA methyltransferase inhibitor 5-azacytidine (Sigma, St. Louis, Mo.) was used at a concentration of 10 μM.

RNA Isolation and Northern BlotAnalysis: Total RNA was isolated from K562 cells by guanidinium isothiocyanate lysis and centrifugation through a cesium chloride cushion. 20 μg of total RNA were subjected to electrophoresis through 1% agarose/2.2 M formaldehyde gels and subsequently transferred to Hybond N⁺ nylon membranes (Amersham Pharmacia Biotech Inc., Piscataway, N.J.). These membranes were then hybridized with radiolabelled probes which specifically interact with the coding region of CD43 mRNA or glyceraldehyde-3-phosphate dehydrogenase (GAPDH) mRNA. The GAPDH and CD43 coding region probes have been described previously (Shelley et al., (1989) Proceedings of the National Academy of Sciences USA, 86:2819-2823; Tso et al., (1985) Nucleic Acids Research, 13:2485-2502). Probes were labeled with (α³²P)-dCTP using a NonaPrimer Kit (Appligene, Illkirch, France). Following hybridization, membranes were washed and subjected to autoradiography.

Southern BlotAnalysis. DNA was subjected to electrophoresis through a 1% agarose gel and transferred to a Hybond N⁺ nylon membrane (Amersham Pharmacia Biotech Inc., Piscataway, N.J.). This membrane was then hybridized with the oligonucleotide LUC-2 which had been radiolabeled at its 5′ end and which specifically interacts with the coding region of the firefly luciferase gene. LUC-2 was labeled using T4 polynucleotide kinase and [γ³²P]ATP. The nucleotide sequence of LUC-2 is: 5′-ATAGCCTTATGCAGTTGCTCT-3′ (SEQ ID NO: 23). Following hybridization, membranes were washed and subjected to autoradiography.

Plasmid Construction: The activity of the CD43 promoter was assessed using the expression vector pATLuc (Shelley et al., (1993) Proceedings ofthe National Academy of Sciences USA, 90:5364-5368) which contains a promoterless firefly luciferase reporter gene. The polymerase chain reaction (PCR) was used to generate a fragment of the CD43 gene representing nucleotides −2 to +99 relative to the most 5′ of the two major transcription initiation sites (Shelley et al., (1990) Biochemical Journal, 270:569-576). This fragment was then subcloned into the “filled-in” HindIII site of pATLuc to generate p43Wt (Shelley et al., (2001) Br Journal of Haematology, 115:159-166; Farokhzad et al., (2000) Nucleic Acids Research, 28:2256-2267). The correct orientation and nucleotide sequence of the CD43 promoter within p43Wt was verified by DNA sequencing (Sanger et al., (1977) Proceedings of the National Academy of Sciences USA, 74:5463-5467). K562 cells carrying within their genome the −2/+99 region of the CD43 gene linked to the luciferase reporter were produced using the plasmid p43Wt/Zeo. This plasmid was generated by inserting between the SalI and PstI sites of p43Wt the XhoI/PstI fragment of pCMV/Zeo (Invitrogen, Carlsbad, Calif.) containing the zeocin resistance gene. Prior to transfection p43Wt/Zeo was linearized by digestion with Pstil such that that the zeocin gene lay downstream and head-to-tail relative to the luciferase gene. The hnRNP-K expression constructs, Full-Length hnRNP-K, GST-RNP-K and the equivalent vectors empty of hnRNP-K coding sequences were kindly provided by David Levens (National Institutes of Health, Bethesda, Md.)(Tomonaga et al., (1995) Journal of Biological Chemistry, 270:4875-4881). The Purα expression construct, pHAPur1, was kindly provided by Edward Johnson (Mount Sinai School of Medicine, New York) and the empty vector equivalent, pHA, produced by religation following liberation of the Purα sequence by RsrII and EcoRI digestion.

Reverse-Transcriptase Polymerase Chain Reaction (RT-PCR). Total RNA was prepared from K562 cells carrying within their genome the CD43 promoter fused to the luciferase reporter. A GeneRacer Kit (Invitrogen Life Technologies, Carlsbad, Calif.) was used to ligate the RNA oligonucleotide GeneRacer RNA Oligo to the 5′ end specifically of full-length mRNA within the total RNA mixture. This ligated mRNA was then converted to cDNA using reverse-transcriptase and the GeneRacer Oligo dTPrimer. Next, this cDNA was used as the template in a PCR with the oligonucleotides GeneRacer 5 ′ Primer, which represents the DNA equivalent of the 5′ end of the GeneRacer RNA Oligo, and LUC-4 which hybridizes to the coding-strand of the luciferase gene. The resulting PCR products were then used as templates in a second round of PCR using the oligonucleotides GeneRacer 5 ′ Nested Primer, which represents the DNA equivalent of the 3′ end of the GeneRacer RNA Oligo, and LUC-2 which hybridizes to the coding-strand of the luciferase gene 5′ to the LUC-4 hybridization site. The products of this second reaction were then cloned using the TOPO TA Cloning Kit for Sequencing (Invitrogen Life Technologies, Carlsbad, Calif.). Clones containing the luciferase coding region were identified by filter hybridization. The radiolabeled probe used in this analysis was a 720 bp EcoRI/PstI fragment isolated from the plasmid p43Wt/Zeo which spans the CD43 promoter and the 5′ end of the luciferase reporter gene. Clones containing the luciferase coding region were analyzed by DNA sequencing. The nucleotide sequences of the oligonucleotides used in RT-PCR were:

GeneRacer RNA Oligo:

(SEQ ID NO: 24)

5′-CGACUGGAGCACGAGGACACUGACAUGGACUGAAGGAGUAGAAA-3′
GeneRacer Oligo dT Primer:

(SEQ ID NO: 25)

5′-GCTGTCAACGATACGCTACGTAACGGCATGACAGTG(T)₁₈-3′.
GeneRacer 5′ Primer:

(SEQ ID NO: 26)

5′-CGACTGGAGCACGAGGACACTGA-3′
GeneRacer 5′ Nested Primer:

(SEQ ID NO: 27)

5′-GGACACTGACATGGACTGAAGGAGTA-3′
LUC-2:

(SEQ ID NO: 23)

5′-ATAGCCTTATGCAGTTGCTCT-3′
LUC-4:

(SEQ ID NO: 28)

5′-CACTACGGTAGGCTGCGAAATGTTCATACTGTT-3′.

Transfection: K562 cells were transfected by electroporation as previously described (Potter et al., (1984) Proceedings of the National Academy of Sciences USA, 81:7161-7165.

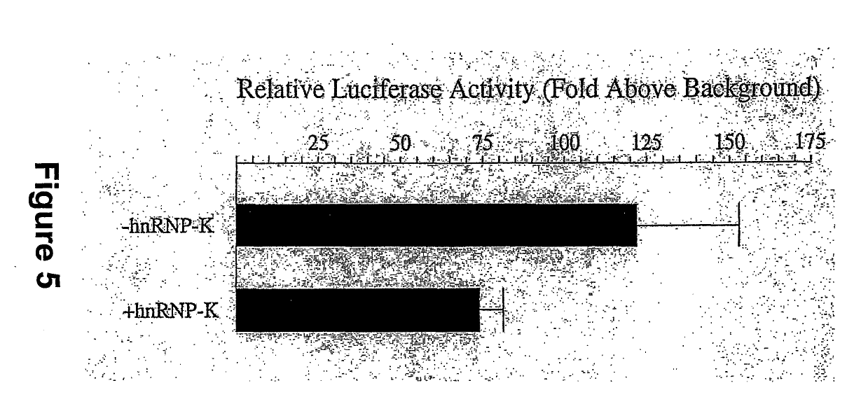

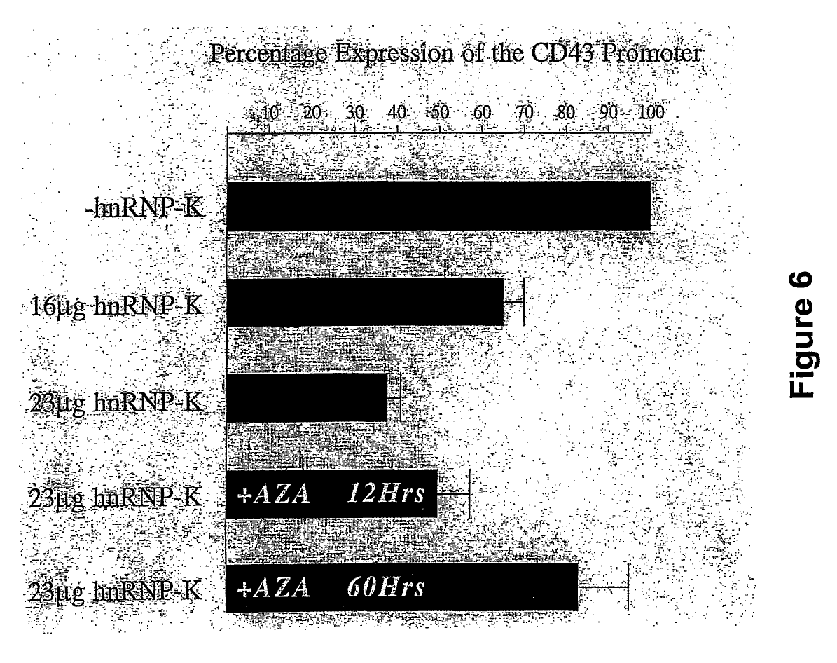

59. Shelley et al., (1991) Proceedings of the National Academy of Sciences USA, 88:10525-10529). Cells were transiently transfected with 23 μg of p43Wt together with 2 μg of the plasmid pRSV-β (Promega Corp., Madison, Wis.) which contains the lacZ gene encoding β-galactosidase. Each transfection of p43Wt was performed in parallel with a transfection of the promoterless luciferase plasmid pATLuc. Transfected cells were then either left untreated or treated with PMA for 12 hours before harvesting and lysis. Luciferase and β-galactosidase activities were subsequently determined using reagents purchased from Promega. Corp. (Madison, Wis.) and Tropix Inc. (Bedford, Mass.), respectively. Luciferase and β-galactosidase activities, assessed as light output, were measured using a Moonlight Luminometer which integrated peak luminescence 10 seconds after injection of assay buffer. The levels of β-galactosidase activity resulting from different transfections were taken as reflective of relative transfection efficiency and used to correct the measurements of luciferase activity. Trans-repression by hnRNp-K in PMA treated K562 cells was assessed by transient transfections in which 8 μg of pATLuc or p43Wt were mixed with 1 μg of pRSV-β and 16 μg of either Full-Length hnRNP-K or the equivalent vector empty of hnRNP-K coding sequences. Full-Length hnRNP-K contains the human hnRNP-K coding region downstream of the cytomegalovirus (CMV) promoter. After correction for transfection efficiency, the level of luciferase activity directed by pATLuc in the presence of Full-Length hnRNP-K was used to divide the levels of luciferase activity directed by p43Wt also in the presence of Full-Length hnRNP-K. This calculation yielded the fold above background activity of p43Wt in the presence of hnRNP-K. Equivalent calculations of luciferase activity in the presence of the CMV vector empty of the hnRNP-K coding region assessed non-specific effects caused by the lo vector backbone. The results (FIGS. 5 and 6) from this second set of calculations represented the fold above background activity of p43Wt in the absence of hnRNP-K. K562 cells carrying stably within their genome the CD43 promoter linked to the luciferase reporter were transfected with 2 μg of pRSV- β and 23 μg of either Full-Length hnRNP-K or the equivalent vector empty of hnRNP-K coding sequences.

Affinity Purification: 200 μg of Streptavidin MagneSphere paramagnetic particles (Promega, Madison, Wis.) were washed two times in 1 ml of phosphate buffered saline (PBS) containing 0.1% bovine serum albumin (BSA) then mixed on a rotating wheel for 30 min with 1 ml of 0.2 M NaCl containing 10 μg of a version of the oligonucleotide CD43 PyRo SS (Farokhzad et al., (2000) Nucleic Acids Research, 28:2256-2267) which was biotinylated at its 3′ end. The particles were then washed twice in 1× washing buffer (75 mM NaCl, 20 mM Tris-HCl pH 7.5, 1 mM EDTA, 15% glycerol, 0.05% IGEPAL CA-630). DNA affinity particles were mixed for 30 min at room temperature on a rotating wheel with 500 μl of binding mixture consisting of 1× binding buffer (70 mM KCl, 5 mM NaCl, 20 mM Tris-HCl pH 7.5, 0.5 mM EDTA, 1 mM DTT and 10% glycerol), 300 μg of nuclear extract prepared from Jurkat cells and 0.2 μg/l poly(dI-dC). The particles were captured with a magnetic stand and washed three times with 1 ml of 2× binding buffer containing 0.2 μg/μl poly(dI-dC). Bound protein was then eluted by adding 50 μl of washing buffer containing 1 M NaCl. The eluted protein was then subjected to EMSA and SDS-PAGE analysis.

Protein Identification by MALDI-TOF-MS: Affinity purified protein from Jurkat nuclear extracts was subjected to SDS-PAGE and stained with Coomassie blue. The stained band was excised from the gel and then cut into small but uniform pieces. The gel was dehydrated with acetonitrile and rehydrated with 100 mM ammonium bicarbonate. Protein was protected from oxidation by incubation at 56° C. for 1 hour with 10 mM DTT and the amino terminus protected by treatment with 10 mM iodoacetamide in 100 mM ammonium bicarbonate. Next, gel pieces were subjected to two rounds of washing with ammonium bicarbonate and subsequent drying with acetonitrile followed by a 12 hour incubation at 37° C. with 12.5 ng/μl of trypsin in 50 mM ammonium bicarbonate (Rosenfeld etal., (1992) Analytical Biochemistry, 203:173-179; Wilm et al., (1996) Analytical Chemistry, 68:1-8). The masses of the trypsin-digested peptides were determined by matrix assisted laser desorption/ionization time-of-flight mass spectroscopy (MALDI-TOF-MS) using a Voyager DE-PRO instrument (Perceptive Biosystems Inc., Framingham, Mass.). Protein identification was achieved by mass fingerprinting using the Mascot database created by Matrix Science.

Expression and Purification of Recombinant GST Proteins: The construct GST-RNP-K and its parent vector empty of hnRNP-K sequences were introduced into the E. coli strain XL-2 (Stratagene, La Jolla, Calif.). Bacteria were grown in 50 mls of Lauria broth at 37° C. until they reached an optical density at 600 nm of 0.6 then recombinant protein expression was induced for 7 hours with 0.5 mM isopropyl β-D-thiogalactopyranoside (IPTG). Bacteria were washed twice in PBS and resuspended in 1 ml of 1× binding buffer (70 mM KCl, 5 mM NaCl, 20 mM Tris-HCl pH 7.5, 0.5 mM EDTA, 1 mM DTT and 10% glycerol) containing “Complete” proteinase inhibitors (Roche Diagnostics Corp., Indianapolis, Ind.). Bacterial lysates were prepared by sonication and clarified by centrifugation. These preparations were then incubated for 30 min at room temperature with 200 μl of Glutathione-Sepharose beads (Amersham Pharmacia Biotech, Piscataway, N.J.). Beads were washed three times with, 1 ml of PBS containing “Complete” inhibitors and bound proteins eluted by adding 800 μl of 50 mM Tris-HO, pH 8.0, 5 mM reduced glutathione. Finally, proteins were concentrated by centrifugation on a centricon-30 filter unit (Millipore Corp., Bedford, Mass.) for 15 min at 5000 g.

Electrophoretic Mobility ShiftAssay (EMSA): Oligonucleotides were labeled using T4 polynucleotide kinase and purified through Micro Bio-Spin 6 columns (Bio-Rad Laboratories, Hercules, Calif.). DNA-protein binding reactions were carried out in a 20 μl volume. Protein preparations were incubated with or without a molar excess of unlabeled specific or non-specific competitor oligonucleotides at 4° C. for 10 min in 1× binding buffer (70 mM KCI, 5 mM NaCl, 20 mM Tris-HClpH 7.5, 0.5 mM EDTA, 1 mM DTT and 10% glycerol) with 2.4 μg/μl poly(dI-dC). Radiolabeled oligonucleotides were then added and the incubation continued for 20 min. DNA-protein complexes were resolved by electrophoresis through 4 or 6% polyacrylamide gels with 0.5×TBE and visualized by autoradiography. The oligonucleotides used in the EMSA analyses were:

CD43 PyRo SS (Farokhzad et al., (2000)
Nucleic Acids Research, 28:2256-2267):

| | |
|---|---|
| 5′-GGGCCCACTTCCTTTCCCCTTG-3′ | (SEQ ID NO: 19) |

CD43 PyRo SSUB:

| | |
|---|---|
| 5′-GGGCCCACUUCCUUUCCCCUUGB-3′ | (SEQ ID NO: 20) |

(U=Bromouracil; B=Biotin).

CD43 Mut-11 (Farokhzad et al., (2000) Nucleic Acids Research, 28:2256-2267):

| | |
|---|---|
| 5′-GGGCCCACTTCCTTCATATATG-3′ | (SEQ ID NO: 21) |

NS-SS (Farokhzad et al., (2000) Nucleic Acids Research, 28:2256-2267):

| | |
|---|---|
| 5′-GAGTTAGCTCACTCATTAGG-3′ | (SEQ ID NO: 22) |

UV Cross-Linking: UV crosslinking was performed using the oligonucleotide CD43 PyRo SSUB. This oligonucleotide is the same as the PyRo1 binding probe CD43 PyRo SS (Farokhzad et al., (2000) Nucleic Acids Research, 28:2256-2267) except that it is biotinylated at its 3′ end and its deoxythymidines have been substituted with 5-bromo-2′-deoxyuridine. 30 μg of Jurkat nuclear extract were preincubated for 5 minutes in 50 μl of EMSA binding buffer containing 2.4 μg/μl poly(dI-dC) then 10⁵ cpm of radiolabeled CD43 PyRo SSUB were added and binding performed for 20 minutes on ice in 1.5 ml microcentrifuge tubes. The tubes were then opened and placed in a UV crosslinker (Stratagene, La Jolla, Calif.) and exposed to 254 nm UV light for 10 minutes. Next 100 μl of washed streptavidin-coated magnetic particles were added and crosslinked CD43 PyRo SSUB-protein complexes captured by rotation for 20 minutes at room temperature. Streptavidin particles were sedimented with a magnetic stand, washed five times with 500 μl of EMSA binding buffer and then resuspended in 25 μl of EMSA buffer containing 2% SDS and 5% 2-mercaptoethanol. Samples were boiled for 5 minutes, centrifuged to pellet the magnetic particles and the supernatant analyzed by electrophoresis through a 12% SDS-polyacrylamide gel and subsequent autoradiography.

Results

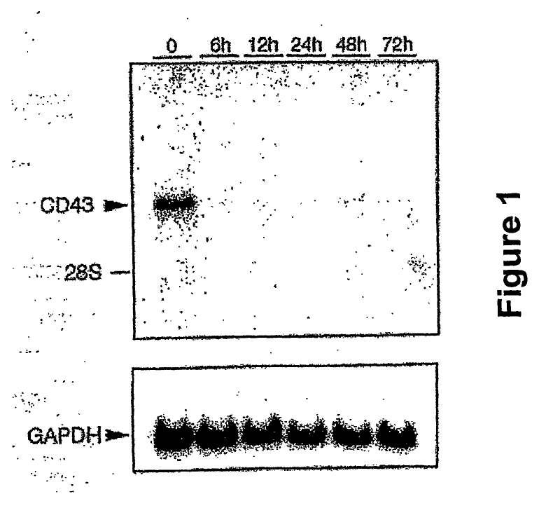

Down-Regulation of CD43 mRNA Levels During K562 Activation: During activation leukocytes increased their adhesive capacity mediated in part by a down-regulation of CD43 expression. We have shown that 48 hours after activation of the monocytic cell line U937 with phorbol ester there was a 69% reduction in CD43 mRNA levels (Shelley et al., (2001) Br Journal of Haematology, 115:159-166). A more dramatic reduction in CD43 mRNA levels followed activation of the pre-erythroid/pre-megakaryocytic cell line K562 (FIG. 1).

Total RNA was prepared from K562 cells treated with PMA for the times indicated. RNA was then subjected to northern blot analysis using an EcoRI/NcoI fragment (CEM-E/N) isolated from the CD43 cDNA clone pCEMI.7 which has previously been used to detect CD43 mRNA (Shelley et al., (1989) Proceedings of the National Academy of Sciences USA, 86:2819-2823). As a control for RNA loading, the same northern blot hybridized with the CD43 specific probe was subsequently hybridized with a probe which specifically recognizes GAPDH mRNA (Tso et al., (1985) Nucleic Acids Research, 13:2485-2502). Within 6 hours of K562 cells being activated with phorbol ester the steady-state levels of CD43 mRNA become barely detectable.

Repression of CD43 Promoter Activity During K562 Activation: Previous studies have indicated that the cis-acting elements responsible for directing the leukocytic expression of the CD43 gene may be located in the proximal promoter region (Shelley et al., (2001) Br Journal of Haematology, 115:159-166; Farokhzad et al., (2000) Nucleic Acids Research, 28:2256-2267; Kudo et al., (1991) Journal of Biological Chemistry, 266:8483-8489; Kudo etal., (1994) European Journal of Biochemistry, 223:319-327). The construct p43Wt containing nucleotides −2 to +99 of the CD43 gene promoter was transfected into K562 cells along with the control plasmid pRSV-β encoding β-galactosidase. Transfected cells were then either left untreated (−PMA) or treated for 12 hours with PMA (+PMA) prior to harvesting. Luciferase values were measured and normalized against β-galactosidase levels to correct for transfection efficiency.

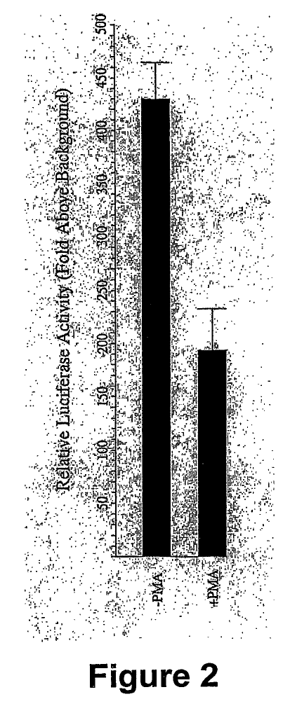

The transfection experiments (employing the expression construct p43Wt which contains the CD43 promoter spanning nucleotides −2 to +99) indicated that the proximal promoter also contained the elements required for CD43 repression during K562 activation. Specifically, we found that expression of p43Wt was repressed by 39% upon PMA mediated activation of K562 cells (FIG. 2). Expressed as histograms are the levels of luciferase gene activity directed by p43Wt in untreated and PMA treated K562 cells after division by the background activity conferred by the control plasmid pATLuc. Each histogram represents the mean±the standard deviation of three independent transfection experiments.

Identification of a Putative Repressor of the CD43 Promoter: Initially we have set out to determine the relationship between PyRo1 and hnRNP-K. We have discovered herein that PyRo1 and hnRNP-K are likely the same molecule (or at least structurally similar) and that these molecules are useful for repressing the CD43 promoter. In order to determine the function of PyRo1 we undertook its molecular cloning. PyRo1 was purified from Jurkat T-lymphocytic cells by affinity capture, digested with trypsin and its resulting peptides subjected to analysis by mass spectrometry. The fragmentation patterns produced from the PyRo1 peptides were then compared to those of known proteins deposited in the Mascot database created by Matrix Science. The most significant match to a known human protein was with heterogeneous nuclear ribonucleoprotein K (hnRNP-K) (Matunis et al., (1992) Molecular and Cellular Bioloy, 12:164-171). Next, a fusion protein of hnRNP-K and glutathione S-transferase (GST) was produced in bacteria.

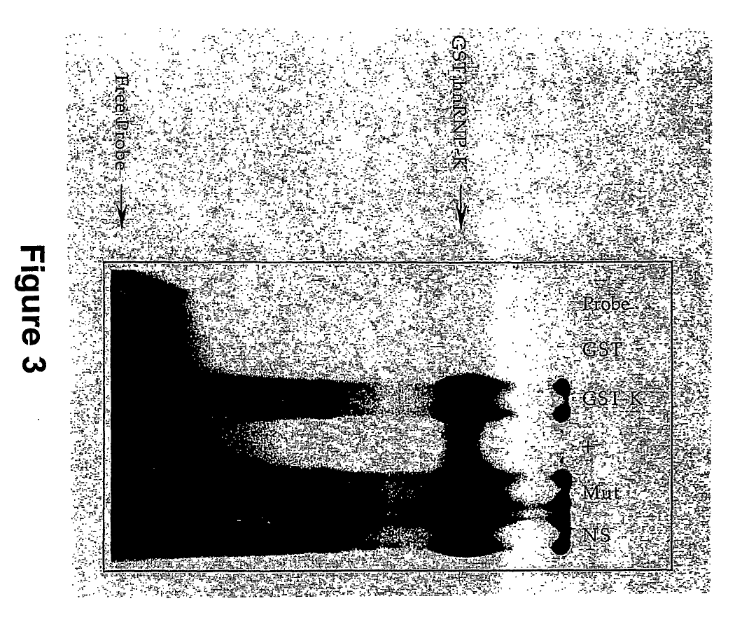

FIG. 3 shows a radiolabeled single-stranded oligonucleotide, CD43 PyRo SS (Farokhzad et al., (2000) Nucleic Acids Research, 28:2256-2267), representing nucleotides +18 to +39 of the sense strand of the CD43 gene was incubated with no protein (Probe), purified GST (GST) or purified GST/hnRNP-K fusion protein (GST-K). Binding reactions containing GST/hnRNP-K were performed in the absence (GST-K) or presence (+) of a 100 fold molar excess of unlabeled CD43 PyRo SS, the presence of Mut-11 (Mut) representing a mutant version of CD43 PyRo SS which fails to support PyRol binding Farokhzad et al., (2000) Nucleic Acids Research, 28:2256-2267) or the presence of an unrelated oligonucleotide (NS). The free probe and probe bound by the GST/hnRNP-K protein (GST:hnRNP-K) are arrowed.

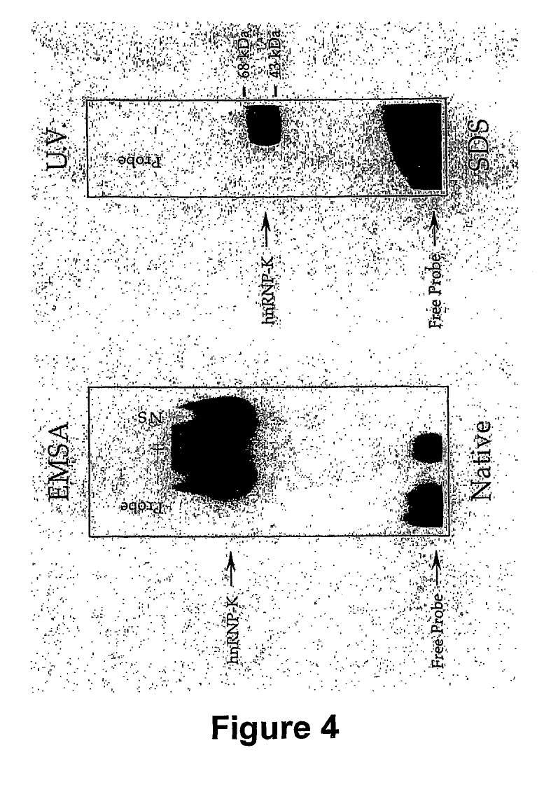

The fusion protein was purified and shown to bind a radiolabeled single-stranded oligonucleotide representing the PyRo1 binding site within the CD43 gene promoter (FIG. 3). This binding was effectively competed by an unlabeled excess of the PyRo1 binding site. However, binding failed to be competed with an identical molar excess of a mutant version of the binding site which previously failed to support PyRo1 interaction (Farokhzad et al., (2000) Nucleic Acids Research, 28:2256-2267). These studies demonstrate that the DNA binding characteristics of PyRo1 and hnRNP-K are similar. In addition, UV crosslinking demonstrated that the apparent molecular mass of PyRo1 is in the range of 50-65 kDa (FIG. 4) which is comparable to that reported for hnRNP-K (Dejgaard et al., (1994) Journal of Biological Chemisty, 236:33-48).

The left panel in FIG. 4 shows the EMSA analysis performed as described in FIG. 3 except binding reactions contained the radiolabeled oligonucleotide CD43 PyRo SSUB which is both bromouracil and biotin modified and either no protein extract (Probe) or a nuclear extract prepared from Jurkat cells. Binding reactions containing Jurkat nuclear extract were performed in the absence (−) or presence (+) of a 100 fold molar excess of unlabeled CD43 PyRo SS (Farokhzad et al., (2000) Nucleic Acids Research, 28:2256-2267) or the presence of an unrelated oligonucleotide (NS). Marked with arrows are the positions of migration through a native polyacrylarnide gel of CD43 PyRo SSUB unbound by protein (Free Probe) and bound by hnRNP-K. This analysis established that the protein binding characteristics of CD43 PyRo SSUB are indistinguishable from its equivalent CD43 PyRo SS which is modified neither with bromouracil nor biotin (Parokhzad et al., (2000) Nucleic Acids Research, 28:2256-2267) (right panel of FIG. 4). The oligonucleotide CD43 PyRo SSUB was radiolabeled, incubated with (−) or without (Probe) a nuclear extract prepared from Jurkat cells and then exposed to Lw light. CD43 PyRo SSUB was captured by streptavidin-coated magnetic particles, washed and subjected to electrophoresis through a 12% SDS-polyacrylamide gel and autoradiography.

Originally, hnRNP-K was identified as a RNA binding protein and recently its consensus RNA binding site was determined as UC₃₋₄(U/A)₂ (Matunis et al., (1992) Molecular and Cellular Biology, 12:164-171; Thisted et al., (2001) Journal of Biological Chemistry, 276:17484-17496). The single-stranded DNA equivalent of this consensus is present in the PyRo1 binding site within the CD43 promoter and mutation analysis has demonstrated that this sequence is critical for PyRo1 binding (Farokhzad et al., (2000) Nucleic Acids Research, 28:2256-2267). Consequently, by the criteria of molecular cloning, molecular mass analysis and nucleic acid binding characteristics PyRo1 and hnRNP-K are structurally similar.

Repression of the CD43 Gene Promoter by hnRNP-K. We sought to determine if hnRNP-K could indeed function in such a capacity. Transfection of 16 μg of a plasmid expressing hnRNP-K indicated that in PMA treated K562 cells hnRNP-K did indeed represses the transcriptional activity of p43Wt. The level of this repression averaged 38% (FIG. 5).

Eight micrograms of the luciferase reporter construct p43Wt were transfected into K562 cells mixed with either 16 μg of Full-Length hnRNP-K which expresses hnRNP-K or 16 μg of its parent vector empty of hnRNP-K coding sequences. One microgram of the β-galactosidase expression plasmid pRSV-β was also included in each transfection to control for transfection efficiency. Transfected cells were treated with PMA for 12 hours, harvested and luciferase and galactosidase assays performed. The levels of β-galactosidase activity were taken as reflective oftransfection efficiency and used to correct the luciferase assay results. Depicted as histograms are the levels of luciferase activity directed by p43Wt divided by those directed by the empty vector pATLuc in the presence of Full-Length hnRNP-K (+hnRNP-K) or the equivalent empty vector (−hnRNP-K). The means of these levels±the standard deviation resulting from three independent experiments are displayed in FIG. 5.

It has been reported that the affects of hnRNP-K on transcriptional activity are dependent upon chromosomal structures (Lau et al., (2000) Journal of Cellular Biochemistry, 79:395-406). Consequently, we generated a pool of K562 cell lines in which the −2/+99 CD43 promoter linked to the luciferase reporter was randomly integrated into the genome.

K562 cells were transfected with the linearized plasmid p43Wt/Zeo, which was derived from p43Wt by insertion of the zeocin resistance gene. K562 cells in which the p43Wt/Zeo plasmid was stably integrated within the genome were selected by treatment with zeocin. The mixed pool of zeocin resistant cells were then transfected with 2 μg of pRSV-β mixed with either 23 μg of Full-Length hnRNP-K or 23 μg of its parent vector empty of hnRNP-K coding sequences. Transfections were also performed using 9 μg of pRSV-β mixed with either 16 μg of Full-Length hnRNP-K or 16 μg of its parent. In those experiments in which the role of DNA methylation was assessed, cells were either untreated or pretreated for 48 hours with 5-azacytidine prior to transfection with 2 μg of pRSV-β mixed with either 23 μg of Full-Length hnRNP-K or 23 μg of its parent. Following all transfections cells were treated with PMA for 12 hours prior to harvesting and assay of luciferase and β-galactosidase activities. In experiments assessing the influence of DNA methylation, transfected cells, untreated or pretreated with 5-azacytidine, were treated simultaneously with PMA and 5-azacytidine for 12 hours prior to harvesting. The levels of β-galactosidase activity were taken as reflective of transfection efficiency and used to correct the luciferase assay results. The level of luciferase activity directed by the CD43 promoter in the presence of the vector empty of HnRNP-K coding sequences (−hnRNP-K) was assigned a value of 100%. The luciferase levels directed by the CD43 promoter matched, parallel transfections employing Full-Length hnRNP-K (+hnRNP-K) was calculated as a proportion of the 100% value. The mean of these proportional values±the standard deviation resulting from three independent experiments are displayed in FIG. 6.

Transfection of the hnRNP+-K expression plasmid into this stable cell line subsequently treated with PMA repressed luciferase reporter gene activity by an average of 62% (FIG. 6). Consequently, hnRNP-K significantly represses the CD43 promoter either in a chromosomal or extra chromosomal context.

Repression Mediated by hnRIP-K is Dependent Upon DNA Methylation. Previous studies have demonstrated that in non-hematopoietic cells the CD43 promoter is maintained in an inactive state by DNA methylation (Kudo et al, (1995) Journal of Biological Chemistry, 270:13298-13302; Kudo et al., (1998) Molecular and Cellular Biology, 18:5492-5499). We tested whether hnRNP-K repression of the CD43 promoter was methylation dependent. The pool of stable cell lines containing the CD43 promoter linked to luciferase was treated for 48 hours with the DNA methytransferase inhibitor 5-azacytidine (Creusot et al., (1982) Journal of Biological Chemistry, 257:2041-2048). These treated cells were then transfected with the hnRNP-K expression plasmid and incubated for 12 hours in the presence of PMA and 5-azacytidine. Under these circumstances the CD43 promoter was repressed by only 17% (FIG. 6). Without a 48 hour pretreatment but with a 12 hour treatment with 5-azacytidine and PMA immediately after transfection hnRNP-K repressed the CD43 promoter by 50%. In cells treated only with PMA after transfection hnRNP-K represses the CD43 promoter by 62% (FIG. 6). These results indicate that exposure of the hnRNP-K expression plasmid to 5-azacytidine for 12 hours influences its ability to effect repression by only 12%. In contrast, exposure of the CD43 promoter to 5-azacytidine for 48 hours reduces the repressive capacity of hnRNP-K by 33%. Consequently, repression of the CD43 promoter by hnRNP-K is at least partially affected by this promoter being methylated.

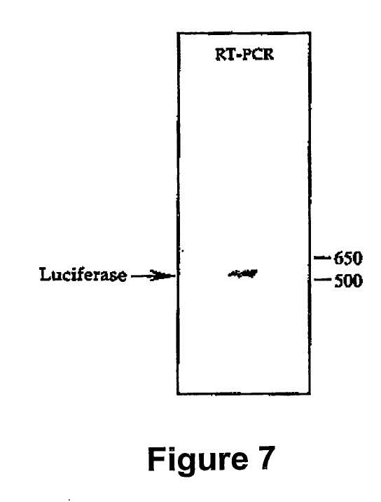

Determination of the Transcription Start Site of the CD43 Gene when Linked to Luciferase and Integrated within the Genome ofK562. Transcription of the CD43 gene has been shown to be initiated at two major sites (Shelley et al., (1990) Biochemical Journal, 270:569-576) between which lies the binding site for hnRNP-K. hnRNP is known to bind RNA and has been implicated in mediating RNA stability and transport (Pi-ol-Roma et al., (1992) Nature, 355:730-732; Pi-ol-Roma et al., (1993) Trends in Cell Biology, 3:151-155; Michael et al., (1995) Cold Spring Harbor Symp Quant Biology., 60:663-668; Kiledjian et al., (1995) EMBO Journal., 14:4357-4364; Visa et al., (1996) Cell, 84:253-264; Michael et al., (1997) EMBO Journal, 16:3587-3598; Dreyfuss et al., (2002) Nature Reviews: Molecular Cell Biology, 3;1 95-205). Consequently, if transcription of the CD43/luciferase gene is initiated at the upstream of the two potential start sites, the affect of hnRNP-K on luciferase activity may reflect mechanisms mediated by RNA binding rather than transcriptional control mechanisms. Due to this reasoning, we identified the transcription start site of the CD43/luciferase gene present in the genome of K562 using RT-PCR coupled with RACE technology. Southern blot analysis of the RT-PCR products resulting from this procedure revealed a single discrete cDNA containing the luciferase coding region (FIG. 7). Cloning and sequencing of this cDNA determined that the downstream of the two potential transcription start sites is utilized by the CD43 promoter when linked to luciferase and integrated within K562. Therefore, the transcript produced from this promoter does not contain a binding site for hnRNP-K. Consequently, repression of the CD43/luciferase fusion gene by hnRNP-K is likely mediated by transcriptional mechanisms as opposed to mechanisms involving RNA binding such as decreased RNA stability and/or transport.

The mixed pool of K562 cells containing within their genome the CD43/luciferase fusion gene were treated for 24 hours with PMA and then total RNA isolated using an RNeasy Maxi Kit (Qiagen, Inc., Valencia, Calif.). A GeneRacer Kit (Invitrogen Life Technologies) was employed to ligate the GeneRacer RNA Oligo specifically to the 5′ end of full-length mRNA which was then reverse-transcribed using the GeneRacer Oligo dT Primer. The resulting products were used as templates in a PCR containing the primer LUC-4, which hybridizes to the coding strand of the luciferase gene, and the GeneRacer 5′ Primer, which represents the DNA equivalent of the 5′ end of the GeneRacer RNA Oligo. PCR products were then subjected to Southern blot analysis using as probe the radiolabeled oligonucleotide LUC-2 which hybridizes to the coding strand of the luciferase gene further 5′ than does LUC-4. The hybridization signal detected by autoradiography is depicted in FIG. 7.

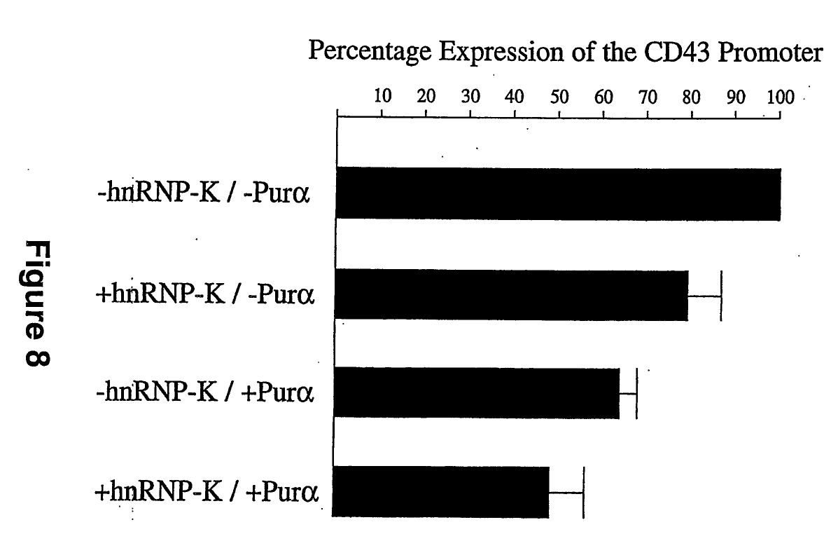

HnRNP-KActs Together with Purα to Effect Repression of the CD43 Promoter. We have previously demonstrated that Purα interacts with the CD43 promoter immediately upstream ofthe region which we show here binds hnRNP-K (Shelley et al., (2001) Br Journal of Haematology, 115:159-166). In the pro-monocytic cell line U937 treated with PMA Purc represses the CD43 promoter. Therefore, it was of interest to determine whether Purα also mediates repression within K562 cells and whether it works with or against hnRNP-K. In transfection experiments employing expression plasmids constitutively expressing hnRNP-K or Purα we found that alone Purα represses the CD43/luciferase gene present within the K562 genome by 36% (FIG. 8). However, when recombinant Purα is expressed together with recombinant hnRNP-K repression of the CD43 promoter is increased to 51%. Consequently, hnRNP-K and Purα have the ability to work together to effect repression.

The β-galactosidase expression construct pRSV-β was mixed with one of four different combinations of plasmids. First, pHAPur1 and Full-Length hnRNP-K which express Purα and hnRNP-K, respectively. Second, pHAPur1 and the empty vector equivalent of Full-Length hnRNP-K. Third, Full-Length hnRNP-K and pHA which represents the empty vector equivalent of pHAPur1. Fourth, both empty vectors. These four DNA mixtures were then transfected into the mixed pool of K562 cells containing the CD43/luciferase fusion and treated for 12 hours with PMA. Cells were harvested, lysed and luciferase and β-galactosidase assays performed. The levels of β-galactosidase activity were taken as reflective of transfection efficiency and used to correct the luciferase assay results. Using these corrected values, the level of luciferase activity directed by the CD43 promoter in the presence of the empty vectors was assigned a value of 100%. The levels of luciferase activity directed by the promoter in the presence of constructs expressing Purα and/or hnRNP-K was calculated as a proportion of the 100% value. The mean of these levels+the standard deviation resulting from three independent experiments are displayed as histograms (FIG. 8).

On the surface of resting leukocytes CD43 prevents cellular interaction. However, during leukocyte activation the glycosylation pattern of CD43 changes and there is a down-regulation of the overall amount of CD43 expressed on the cell surface. These alterations in both the qualitative and quantitative expression of CD43 result in the promotion of both homotypic and heterotypic leukocyte interactions. Using in vitro models of leukocyte activation we have found that down-regulation of CD43 is mediated by repression of the transcriptional activity of the CD43 gene promoter. Previously, we demonstrated that the transcription factor Purα mediates repression of the CD43 promoter (Shelley et al., (2001) Br Journal of Haematology, 115:159-166). We have found according to some aspects of the invention that hnRNP-K also mediates CD43 gene repression. The binding sites for Purα and hnRNP-K lie adjacent to one another within the CD43 promoter. Purα interacts with the −2/+17 region of the CD43 gene and hRNP-K interacts with the +18/+39 region. Both factors are induced during U937 activation concomitant with repression of CD43 promoter activity.

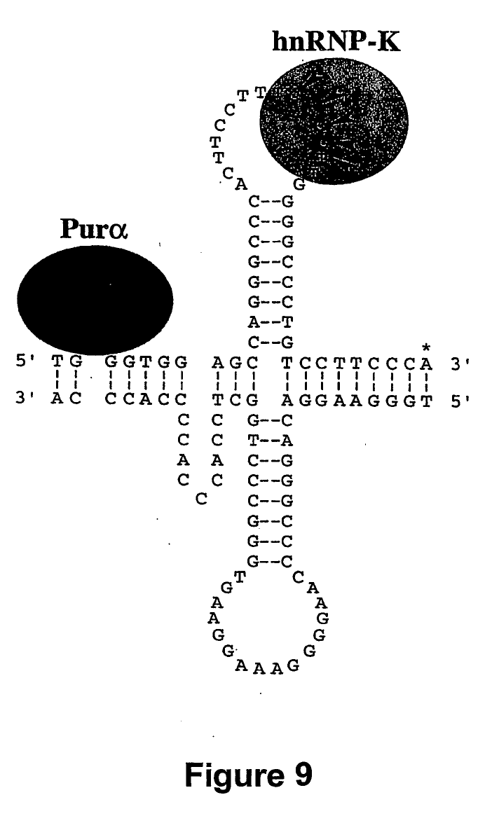

Purα and hnRNP-K interacted preferentially with single-stranded DNA indicating that single-stranded structures within the CD43 promoter are probably important in regulating its activity. Nuclease S1 analysis has established that single-stranded structures can exist within the CD43 promoter (Farolchzad et al., (2000) Nucleic Acids Research, 28:2256-2267). Furthermore, analysis of the primary sequence of the promoter revealed repeats which could form slippage or cruciform structures (Yagil et al., (1991) Critical Review of Biochemistry Molecular Biology, 26:475-559). The single-stranded loop of a slippage structure within the −2/+17 region would support efficient Purα binding while the loop of a ciuciform structure within the +18/+39 region would support efficient binding of hnRNP-K (FIG. 9). The hnRNP-K protein was originally characterized as an RNA binding protein.

FIG. 9 shows the misalignment of direct repeats of the sequence GGTGG may form slippage structures at the 5′ end of the promoter. The single-stranded region on the sense-strand of these structures would support efficient binding of Purα. Two types of slippage structures are possible. One is illustrated. Pairing of an inverted repeat of the sequence CAGGGCCC could form a cruciforni structure immediately downstream of the slippage structures. This possible cruciforni is illustrated. The single-stranded loop region on the sense strand of the cruciform would support efficient binding of hnRNP-K. The DNA equivalent of the consensus RNA binding site of hnRNP-K is marked by bold characters (Thisted et al., (2001) Journal of Biological Chemistry, 276:17484-17496). The nucleotides defmed by mutation analysis as being important for hnRNP-K binding are marked with arrows. The two major transcription initiation sites of the CD43 gene are marked with asterisks.

The site within the CD43 promoter which binds hnRNP-K lies adjacent to a site which interacts with Spl (Shelley et al., (2001) Br Journal of Haematology, 115:159-166; Kudo et al., (1994) European Journal of Biochemistry, 223:319-327;. Kudo et al., (1998) Molecular and Cellular Biology, 18:5492-5499). This Spl binding site overlaps the region which binds Purα. Transient transfections have indicated that, unlike hnRNP-K and Purα, Sp1 functions to activate the CD43 promoter 63. Since Sp1 binds double-stranded DNA a mechanism of repression can be envisioned in which a double-stranded CD43 promoter supporting Sp1 binding and transcriptional activation acquires a single-stranded conformation supporting hnRNP-K and Purα binding. Such opening of the CD43 promoter may in fact be mediated by Purα and hnRNP-K themselves. This possibility was supported by the finding that Purα is capable of displacing an oligonucleotide annealed to single-stranded M13 DNA (Darbinian et al., (2001) Journal of Cellular Biochemistry, 80:589-595). In addition, hnRNP-K has been shown to impart a single-stranded conformation to the CT element of the c-myc promoter contained within a supercoiled plasmid (Michelotti et al., (1996) Molecular and Cellular Biology, 16:2656-2669). However, in the context of the c-myc promoter hnRNP-K acted as a transcriptional activator not a repressor as it did within the CD43 promoter (Michelotti et al., (1996) Molecular and Cellular Biology, 16:2350-2360; Takimoto et al., (1993) Journal of Biological Chemistry, 268:18249-18258). Therefore, while DNA unwinding may be intrinsic properties of Purα and hnRNP-K, the functional consequences of this activity appeared dependent upon promoter architecture and probably also cellular environment.

Previous studies have shown that in non-hematopoietic cells the CD43 gene is maintained in a transcriptionally inactive state by methylation of CpG dinucleotides within the promoter (Kudo et al, (1995) Journal of Biological Chemistry, 270:13298-13302). These methylated dinucleotides are then able to bind the methyl-CpG binding protein MeCP2 (Kudo etal., (1998) Molecular and Cellular Biology, 18:5492-5499). We have shown that repression of the CD43 promoter by hnRNP-K may be affected by DNA methylation. This finding suggests that repression of the CD43 promoter during hematopoietic differentiation may be mediated by an increase in its degree of methylation. Such an increase could result in the recruitment of methyl-CpG binding proteins and/or the more effective recruitment of hnRNP-K.

### Example 2

Introduction

Ovarian cancer is the leading cause of gynecological cancer deaths and the fifth leading cause of all cancer deaths among American women. An encouraging trend of slightly decreasing mortality rates has been due largely to advances in early detection. In contrast, we have failed to significantly advance treatment beyond the sledge-hammer approaches of surgery, chemotherapy and radiotherapy.

Meaningful advances in ovarian cancer treatment will stem only from identifying new diagnostic and prognostic tools and targeting the causative molecular defects.

Recently, we have identified abnormal expression of the sialoglycoprotein CD43 as a novel defect characterizing ovarian cancer tissue. Normal ovarian cells fail to exhibit CD43 expression and no CD43 expression was observed on cells immediately next to the malignant lesions.

CD43 is a large, abundant and highly charged trans-membrane molecule that is normally only expressed on the surface of white blood cells. In this normal context of expression CD43 acts as a barrier molecule preventing intercellular interactions and allowing blood cells to remain in the circulation.

In vitro evidence indicates that, as on white blood cells, CD43 also serves a barrier function on the surface of ovarian cancer cells. In this abnormal context of expression CD43 may prevent interaction with immune effector cells such as natural killers and, therefore, facilitates an escape from immunosurveillance.

As well as allowing malignant cells to avoid immune recognition, expression of CD43 may also contribute to the pathogenesis of ovarian cancer in a another way. By inhibiting cell-cell interactions, CD43 could aid in turning the primary tumor into a loose cellular mass which sheds potentially metastatic neoplastic cells into the circulation.

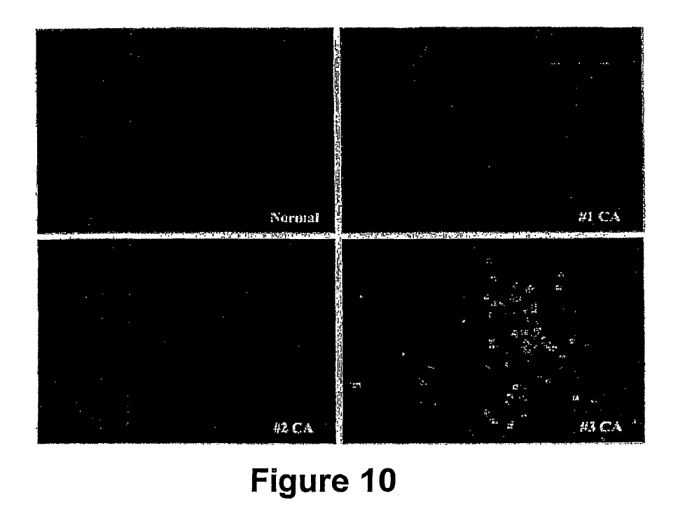

Demonstration that CD43 is Abnormally Expressed in Ovarian Cancer Cells. Human CD43 is a large, abundant and highly charged transmembrane molecule which inhibits intercellular interaction (Remold-O'Donnell, et al., (1990) Immunodefic Rev., 2: 151-174; Ardman et al., (1992) Proc. Natl. Acad. Sci USA, 89: 5001-5005; Manjunath et al., (1993) J. Immunol., 151: 1528-1534). Normally CD43 expression is restricted to the surface of hematopoietic cells. However, we have found that primary ovarian cancer tumors also exhibit CD43 expression. Snap frozen ovarian cancer tissue was obtained from the Massachusetts General Hospital Tumor Bank, sectioned and stained using the monoclonal antibody BS1 which specifically recognizes CD43 independent of its glycosylation status and as secondary antibody donkey anti-Mouse Cy3. The eight specimens analyzed consisted of two endometrial carcinomas, two clear cell carcinomas, two histologically undefined adenocarcinomas and two areas of patient-matched normal ovarian tissue. All the carcinomas stained positive with the BS1 antibody (FIG. 10—panel #1 CA: endometrial ovarian carcinoma; panel #2 CA: clear cell ovarian carcinoma). However, staining of the undefined adenocarcinomas was particularly striking (FIG. 10—panel #3 CA: undefmed ovarian adenocarcinoma). We detected no staining of the patient-matched normal ovarian tissue nor of the tissue surrounding the tumors (FIG. 10—top left panel: normal ovarian tissue from an ovarian cancer patient).

Demonstration that CD43 Expression Protects Cancer Cells From Natural Killing. In 1995 it was demonstrated by the laboratory of Dr. Gunnar Hansson in Gothenburg, Sweden that CD43 is produced by the colon adenocarcinoma cell line COLO205 (Baeckstrom, et al., (1995) I. Rial. Chem., 270: 13688-13692). This was the first identification of a CD43 molecule expressed outside the hematopoietic cell lineage. In 1996 the association of carcinoma with abnormal CD43 expression was confirmed by the laboratory of Dr. Ignacio Molina in Granada, Spain (Santamaria et al., (1996) Cancer Res., 56: 3526-3529). In 1997 the Hansson laboratory published a study which described the functional significance of CD43 expression by carcinoma cells. Returning to their COLO205 model, the Swedish group isolated the CD43 produced by these cells and demonstrated it was able to specifically inhibit cytotoxic lysis mediated by natural killer cells (Zhang et al., (1997) Cell. Immunol., 176: 158-165). Inhibition was concentration dependent and at the highest concentration used (100 μg/ml) reached approximately 60%. This finding indicates that CD43 expression likely contributes to carcinogenesis by helping malignant cells evade immune recognition.

Evasion of immunosurveillance by cancer cells is probably caused by CD43 providing an anti-adhesion shield of protection. Such an anti-adhesion function of CD43 is supported by three sets of experiments. In the first, introduction of CD43 into CD43-negative cervical carcinoma cells was shown to reduce CD54 mediated adhesion (Ardman et al., (1992) Proc. Natl. Acad Sci USA, 89: 5001-5005). In the second, targeted disruption of the CD43 gene in cells derived from acute T-cell leukemia was shown to enhance homotypic adhesion and binding to fibronectin and mv-1 gp120 (Manjunath et al., (1993) I. Immunol., 151: 1528-1534). Finally, in a third study, transgenic mice were produced in which the CD43 gene was specifically knocked-out. These mice exhibited increased leukocyte adhesion and, as a consequence, premature clearance of leukocytes from the circulation (Dragone et al., (1995) Proc. Natl. Acad Sci. USA, 92: 626-630).

Taken together, work from around the world over the last few years has established the anti-adhesion function of CD43. It appears that cancer cells, by abnormally expressing CD43, may acquire this anti- adhesive function and, in so doing, acquire a means of avoiding immunosurveillance.

Although not wishing to be bound to any particular theory or mechanism, we believe that abnormal expression of CD43 in ovarian cancer must be due either to mutation of the gene by which it is encoded and/or alterations in the transcription factors by which this gene is controlled. Our laboratory is in a unique position to identify these molecular defects since over the last decade we have both structurally and functionally characterized the CD43 gene.

Characterization of CD43 cDNA and Genomic Clones Initially we isolated the human CD43 molecule from the T-lymphoblastoid cell line CEM and determined the amino acid sequence of a 15 residue tryptic peptide. An oligonucleotide corresponding to the most frequently used codons specifying this peptide was then used to screen a CEM cDNA library. Sequencing of clones derived from this screen predicted that CD43 is an integral membrane protein with a 19 residue N-terminal hydrophobic leader peptide followed by a 235 residue extracellular region, a 43 residue transmembrane region and a 123 amino acid C-terminal intracellular region (Shelley et al., (1989) Proc. Natl. Acad. Sci. USA, 86: 2819-2823). Southern blot and in situ hybridization analyses established that CD43 is encoded by a single gene located on the p 11.2 band of chromosome 16. This region is often rearranged in patients with acute myelomonocytic leukemia (Pallant et al., (1989) Proc. Natl. Acad. Sci. USA, 86: 1328-1332; Hogge et al., (1984) I. Clin. Oncol., 2: 550-557; LeBeau et al., (1983) N. Engl. J. Med., 309: 630-636).

A human genomic library was screened with CD43 cDNA, a specific clone isolated and the nucleotide sequence of the entire CD43 gene determined (Shelley et al., (1990) Biochem. I., 270: 569-576). The gene is approximately 4.6 kb in length and comprises two exons and one intron. S 1 nuclease protection and primer extension analysis demonstrated that transcription is initiated at two major sites. Upstream of the 5′ major transcription initiation site, 990 nucleotides were sequenced. The promoter region was found to contain a number of short repeats but no TATA or CAAT elements.

The Proximal Promoter Region of the CD43 Gene Directs Tissue-Specific Expression. In order to determine the region of the CD43 gene responsible for driving appropriate cell-specific and tissue-specific expression, a series of deletion constructs were made. The smallest of these plasmid constructs was named p43Wt. This plasmid contains nucleotides −2 to +99 of the CD43 gene fused to the 5′ end of a promoterless luciferase gene. The p43Wt construct was transfected into U937 pro-monocytic cells, Jurkat T-lymphocytes, K562 pre-erythroid/pre- megakaryocytic cells, CEM T-lymphocytic cells and the non-turnorogenic breast epithelial cell line MTSVI-7 (Bartek e. al. (1991) Proc. Natl. Acad. Sci. USA, 88: 25320-2534). Measurement of luciferase activity demonstrated that in U937 cells p43Wt directs expression which is 338 fold higher than that directed by the parent plasmid. Such expression in U937 cells compares favorably to the 309 and 698 fold above parental expression levels directed by p43Wt in Jurkat T -lymphocytes and K562 pre-erythroid/pre-megakaryocytic cells, respectively. When p43Wt was transfected into CEM T-Iyrnphocytic cells it directed a level of expression only 74 fold above that of its parent. This low level of promoter activity compared to other leukocytic cell types reflects the relatively low level of CD43 protein produced by CEM cells (Carlsson et al., (1986) J. Biol. Chem., 261: 12779-12786).

CD43 mRNA is undetectable in the epithelial cell line MTSV 1-7. In this cell line p43Wt directs expression only 7 fold above that directed by the parental plasmid. Our findings differ from those of Kudo and Fukuda who reported the proximal CD43 promoter is active when transfected into epitlielial cells (Kudo et al., (1994) Bur. J. Biochem., 223: 319-327). However, Hela not MTSV1-7 cells were used in these studies. Hela cells are derived from a cervical carcinoma (Gey et al., (1952) Cancer Res., 12: 264-265) while MTSV1-7 cells are derived from normal breast tissue (Bartek et al., (1991) Proc. Natl. Acad Sci. USA, 88: 3520-3524). The development of carcinoma is associated with dramatic changes in DNA methylation (Santini et al., (2001) Ann. Intern. Med., 134: 573-586; Schmutte et al., (1998) Biol. Chem, 379: 377-388). Kudo and Fulcuda have reported that the tissue-specific expression of the CD43 gene is regulated by DNA methylation and its interaction with the methyl-CpG-binding protein MeCP2 (Kudo et al., (1995) J Biol. Chem, 270: 13298-13302;.Kudo et al., (1998) Mol. Cell. Biol., 18: 5492-5499). Therefore, it is possible that the inappropriate expression of the CD43 promoter when transfected into Hela rather than MTSV1-7 cells reflects the disruption of the DNA methylation machinery associated with carcinogenesis.

In summary, we have shown that the region of the CD43 gene extending from −2 to +99 is sufficient in vitro to direct appropriate tissue-specific and cell-specific expression. This finding indicates the importance of analyzing the structure and function of this region in ovarian cancer cells which exhibit inappropriate expression of the CD43 gene.

### Example 3

Experimental evidence generated both in vitro and in vivo indicates that ovarian cancer is characterized by abnormal expression of CD43. Although not wishing to be bound to any particular theory or mechanism, we believe that abnormal expression of CD43 in ovarian cancer contributes in two major ways to disease progression. First, by inhibiting the engagement of ovarian cancer cells by immune effectors such as natural killer cells, CD43 allows ovarian cancer cells to escape immunosurveillance. Second, by inhibiting adhesion, CD43 aids in turning the primary tumor into a loose cellular mass which sheds potentially metastatic neoplastic cells into the circulation. We believe that abnormal CD43 expression is of both diagnostic and prognostic value in ovarian cancer. We also believe that abnormal expression of CD43 in ovarian cancer would be due to abnormal transcription of the gene by which it is encoded.

Determination of the Pattern ofAbnormal CD43 Expression in Ovarian Cancer. Our study of six ovarian cancer tumors has demonstrated that all six exhibit abnormal expression of CD43. This finding has led us to conclude that abnormal CD43 expression is of both diagnostic and prognostic value in ovarian cancer. A series of immunocytochemical examinations using a panel of anti-CD43 antibodies are performed. Each of the distinct biological categories of ovarian tumors (benign, borderline and malignant), each of the histological subtypes (serous, mucinous, endometrioid and clear cell), each of the disease stages (Stage IA -Stage IV) and also BRCA1-associated hereditary ovarian cancer are examined. In addition, we examine a broad range of non-tumorogenic and tumorogenic ovarian cells cultured in vitro. CD43 expression is evaluated in the primary and metastatic tumors which develop in each of seven newly established genetic mouse models of ovarian cancer.

Fluorescence microscopy is used to determine CD43 expression in three types of ovarian samples; sectioned human ovarian tumors, human ovarian cells cultured in vitro and malignant cells generated within seven different genetic mouse models of ovarian cancer.

Human Ovarian Tumors: Fresh ovarian tumors and normal ovarian tissue are obtained from the Gillette Centerfor Women's Cancers-Gynecologic Oncology in collaboration with Arlan F. Fuller, Jr., M. D. and Robert L. DeBernardo, M. D. Snap frozen ovarian tumors and normal ovarian tissue are obtained from the MGH Tumor Bank. Using these sources of material we examine each of the distinct biological categories of ovarian tumors (benign, borderline and malignant), each of the major histological subtypes (serous, mucinous, endometrioid and clear cell) and also BRCAI-associated hereditary ovarian cancer. In addition, we will examine the different. disease stages to determine whether CD43 expression is of prognostic value.

Human Ovarian Cells Cultured in Vitro: Three categories of cultured human ovarian cells are examined; non-tumorogenic primary cultures and cell lines, cystadenoma derived cell lines and ovarian carcinoma derived lines.

Non-Tumorogenic Cell Lines:

(1). Primary cultures established from normal ovarian epithelium (BioWhittaker, Inc., Walkersville, Md.).
(2). Cell lines derived by SV40-immortalization of normal ovarian epithelium; mO-106, mO-118, HIO-121, mo-135 IOSE-80 and IOSE-I44 (Maines-Bandiera et al., (1992) Am. I. Obstet. Gynecol, 167: 729-725; Bruening et al., (1999) Cancer Res., 59: 4973-4983).
(3). Cell lines derived by HPVI6-immortalization of normal ovarian epithelium; HOSE-A and HOSE-B-(Gregoire et al., (1998) In Vitro Cell. Dev. Bioi. Anim., 34: 636-639).
   Cystadenoma-Derived Cell Lines
(1). ML3, ML10, MCV39, MCV50 and MCV152 (Luo et al., (1997) Gynecol. Oncol., 67: 277-284).
   Ovarian Carcinoma-Derived Cell Lines
(1). Primary cultures of ovarian tumors established on a fee-for-service basis by BioWhittaker, Inc. from fresh material supplied by the Gillette Center for Women's Cancers-Gynecologic Oncology.
(2). Caov-3, Caov-4, NIH OVCAR-3, SKOV3, ES-2, PA-1 and SW626 (American Type Culture Collection, Rockville, Md.).
(3). OVCAR-IO, OVCAR-8, OVCAR-5, OVCAR-4, PEO1, PEO4, A1847 and A2780 (Godwin et al., (1992) Proc. Natl. Acad. Sci. USA, 89: 3070-3074).
(4). OVCA433, OVCA420, OVCA429, OVCA432 and DOV!3 (Moser et al., (1996) Int. J. Cancer, 67: 695-701).
(5). HEY, HOC-1, HOC-7 and HOC-8 (Buick et al., (1985) Cancer Res., 45: 3668-3676).
(6). ML9A, ML!2B and ML23A (Luo et al., (1997) Gynecol. Oncol., 67: 277-284).
(7). HOSE-A and HOSE-B exhibiting spontaneous malignant transformation due to extended culture on collagen rafts (Gregoire et al., (2001) Clin. Cancer Res., 7: 4280-4287).

Genetic Mouse Models of Ovarian Cancer: Recently, mouse models of ovarian cancer have been developed in which avian retroviral vectors are used to introduce multiple genetic lesions into ovarian surface epithelial cells (Fisher, et al., (1999) Oncogene 18: 5253-5260; Orsulic et al., (2002) Cancer Cell 1: 53-62; Orsulic (2002) Mamm. Genome, 13: 543-547). To date, the expression of seven combinations of the oncogenes c-myc, K-ras and akt within p53-t-mice have been shown to produce ovarian tumors. The different combinations of oncogenes result in different frequencies of primary tumor development and in tumors with different metastatic potential. We examine CD43 expression on sectioned primary and metastatic tumors in each of the seven genetic models of ovarian cancer.

Staining Procedure: Sectioned human tissue and human cell lines growing on microscope slides are blocked with serum and then incubated with a mouse anti-human CD43 antibody or an isotype matched negative control antibody. A panel of anti-CD43 antibodies is used consisting of L10, L14, DFT1, BRA7G and BS1 which recognize the five different epitope clusters identified on the human CD43 molecule (Remold-O'Donnell (1995) Leukocyte Typing V, 1697-1701;De Smet et al., (1995) Leukocyte Typing V, 1706-1709; Axelsson, et al., (1995) Leukocyte Typing V, 1708-1709). Sectioned mouse tissue is analyzed using the goat anti-mouse CD43 antibody S19 (Santa Cruz Biotechnology, Inc., Santa Cruz, Calif.) and the rat anti-mouse antibodies S7 and 1B11 (PharMingen, San Diego, Calif.). After washing in PBS the cells are incubated with FITC labeled anti-mouse immunoglobulin and observed using a fluorescence microscope. As controls for these experiments immunocytochemical analysis is performed in parallel on CEM cells which express CD43 and on Hela cells which do not. These studies detennine diagnostic and prognostic value of CD43 expression in ovarian cancer.

Determination of CD43 Gene Structure in Ovarian Cancer. Large-scale gene rearrangements and amplifications which result in inappropriate gene expression have been shown to be associated with and, in some cases, to cause malignancy. The presence of gross structural and copy number defects of the CD43 gene in ovarian cancer is determined by Southern blot and/or Polymerase Chain Reaction (PCR) analysis. These analyses are performed on CD43-negative and CD43-positive ovarian cell cultures as well as on CD43-negative and CD43-positive ovarian tissue. On the tissue samples CD43 expression is visualized by immunocytochemistry and CD43-negative and CD43-positive cells isolated by laser capture microdissection (LCM). In Southern blot analysis, cell cultures or material isolated by LCM are embedded in agarose plugs and lysed in SDS. DNA is digested within the agarose plugs with combinations of the restriction enzymes AscI, NotI, PacI, Sce83871 and SfiI. These enzymes have long recognition sequences that occur infrequently within the human genome and, therefore, produce large DNA fragments. Such fragments are resolved by Pulsed Field Gel Electrophoresis (PFGE) and transferred to nitrocellulose filters by Southern blotting (Southern, (1975) I. Mol. Bioi., 98: 503-517). After baking, the filter is hybridized with a ³²p-labeled CD43 cDNA probe, washed and subjected to autoradiography. The human genome database allows probes specific to the CD43 gene locus but distal to the CD43 coding region to be generated by the PCR technique. These probes are also employed in Southern blot analysis. In this way we produce a restriction map of the CD43 gene locus which spans hundreds of kilobases. The hybridization pattern resulting from CD43-positive and CD43-negative material is compared to evaluate chromosomal rearrangements. Quantitation of hybridization signals by densitometry evaluates chromosomal amplification. The large scale of the mapping possible using PFGE allows chromosomal rearrangements distal to the CD43 coding region to be detected. In order to avoid the possibility of detecting non-functional Restriction Fragment Length Polymorphisms hybridization patterns resulting from the digestion of a number of different restriction endonucleases are compared. Differences in patterns in CD43-positive compared to CD43- negative material judge and identify the rearranged CD43 gene locus. This rearrangement is localized by performing Southern blot analysis using restriction enzymes such as Hindll, BamI and EcoRI which have hexanucleotide recognition sequences.

In order to characterize any rearrangement of the CD43 gene detected by Southern blotting and also identify any small scale mutations invisible to this analysis, the CD43 gene is cloned from CD43-positive ovarian cancer cells and sequenced. First, total genomic DNA extracted from ovarian cells expressing CD43 is digested with the restriction enzymes that Southern blot analysis has determined produce fragments spanning the CD43 gene locus. These digests are subjected to agarose gel electrophoresis and the appropriately sized DNA isolated. The isolated fragments are then ligated into plasmid vectors and the CD43 clones, detected by hybridization screening, sequenced by standard means (Shelley et al., (1989) Proc. Natl. Acad. Sci. USA, 86:2819-2823; Ausubel et al., (1994) John Wiley and Sons, Inc.). The sequences of the CD43 genes isolated from CD43-positive ovarian cancer cells are compared with published sequences to identify any mutations. Sequences which cannot be assigned as being of CD43 gene origin due to gross chromosomal rearrangement are identified by BLAST searches of the Genbank nucleotide sequence database. These studies will identify abnormal expression of CD43 in ovarian cancer associated with mutation of the CD43 gene.

Small quantities of genomic DNA can be isolated by LCM. This limitation may preclude Southern blot analysis of CD43 gene structure in CD43-positive tumor tissue. Under these circumstances we evaluate the structure of the CD43 gene by linker modified (LM) PCR. Genomic DNA isolated by LCM is digested with a series of restriction enzymes which cut in or outside the transcribed region of the CD43 gene. Oligonucleotide linkers are then ligated to the cleavage sites and the resulting products are used as templates in PCR. One of the primers employed in these reactions hybridizes to the linker and the other to the known CD43 gene sequence on one side or the other of the restriction enzyme cleavage site. Comparison of the size of the PCR products generated from CD43-positive and CD43-negative tissue assesses rearrangement of the CD43 gene locus. Gene amplification is assessed by real-time quantitative PCR using TaqMan@ probes (Applied Biosystems, Foster City, Calif.).

CD43 gene expression has been shown to be dependent upon its pattern of methylation (Kudo et al., (1995) J. Bioi. Chem., 270: 13298-13302; Kudo (1998) Mol. Cell. Bioi., 18: 5492-5499). Therefore, we compare this pattern in CD43-positive and CD43-negative ovarian cell ines and tissue. Southern blot or PCR analysis is performed in exactly the same way as described above with the exception that specific pairs of restriction endonucleases are employed. These pairs such as MspI and HpaII recognize the same nucleotide sequence but methylation of that sequence blocks digestion by one enzyme but not the other. Consequently, comparison by Southern blot or PCR analysis of the restriction patterns generated by digestion with each of such enzymes reflects the methylation state of the CD43 gene.

Determination of Transcription Factor Structure in Ovarian Cancer. Irrespective of whether the CD43 gene present in ovarian cancer is amplified, mutant or wild-type, deletion and transfection studies are performed to delineate the region responsible for abnormal expression. EMSA, UV crosslinking and cloning are undertaken to characterize the transcription factors with which this region interacts.

Transfection: In ovarian cancer cells where the CD43 gene is rearranged or contains point mutations, progressively smaller restriction fragments spanning these defects are isolated. Such fragments are then cloned in the appropriate orientation upstream of the promoterless luciferase gene present in the plasmid pATLuc (Shelley, et al., (1993) Proc. Natl. Acad. Sci. USA, 90: 5364-5368). Three types of transfection are performed with the resulting constructs using methods we have previously described (Shelley, et al., (1993) Proc. Natl. Acad. Sci. USA, 90: 5364-5368; Farokhzad et al., (1996) I. Immunol., 157: 5597-5605; Shelley et al., (1991) Proc. Natl. Acad. Sci. USA, 88: 10525-10529; Shelley et al., (1992) Leukocyte adhesion 337-351; Bottinger et al., (1994) Mol. Cell. Biol., 14: 2604-2615). The first transfection is into ovarian cancer cell lines which express CD43 and the second is into primary cultures of ovarian tumors established on a fee-for-service basis by BioWhittaker, Inc. These first two transfection studies map to a resolution of approximately 100 bp the region responsible for CD43 gene expression in ovarian cancer. The third set of transfections performed is into normal ovarian surface epithelial cells immortalized by viral infection (Maines-Bandiera et al., (1992) Am. I. Obstet. Gynecol., 167: 729-725; Bruening et al., (1999) Cancer Res., 59: 4973-4983; Gregoire et al., (1998) In Vitro Cell. Dev. Biol. Anim., 34: 636-639) and into primary cultures of normal ovarian cells purchased from BioWhittaker, Inc. These experiments establish if any identified mutation is solely responsible for driving abnormal expression of the CD43 gene in ovarian cells. As a negative control in all these studies mutant constructs are transfected in parallel with p43B which contains the wild-type CD43 gene promoter.

If the CD43 gene is not mutated in ovarian cancer then the defects which cause abnormal CD43 expression may lie in trans to the CD43 gene. In order to induce transcription of the CD43 gene, ultimately these trans-acting defects must alter the nature of transcription factor interaction with the CD43 gene. The region of the CD43 gene where these altered interactions occur is mapped by cloning progressively smaller fragments of the wild-type CD43 promoter into pATLuc. The resulting constructs is methylated in vitro to mimic any cancer specific methylation patterns detected by Southern blotting (Kudo et al., (1995) J. Biol. Chem., 270: 13298-13302) and transfected into ovarian cell lines and primary cultures. In the event that primary ovarian cultures cannot be transfected either by electroporation or other methods such as those employing liposomes, calcium phosphate and DEAE dextran, virus infection is employed (Kolanus et al., (1992) EMBO I., 11: 4861-4868;Aftringetal. (1994) Hepatology, 19: 1054-1056; Martiniello-Willcs et al., (1998) Hum. Gene Ther., 9: 1617-1626).

EMSA: The experiments outlined above define to a resolution of approximately 100 bp the regions responsible for CD43 gene expression in ovarian cancer. A series of overlapping single and double stranded oligonucleotides covering these regions are synthesized and used in EMSA analyses to characterize the transcription factors with which they interact. Using methods we have previously described, nuclear extracts are prepared from ovarian cancer cell lines and primary cultures as well as from immortalized and primary normal ovarian cultures (Shelley, et al., (1993) Proc. Natl. Acad. Sci. USA, 90: 5364-5368; Farokhzad et al., (1996) I. Immunol., 157: 5597-5605; Shelley et al., (1992) Leukocyte adhesion 337-351; Bottinger et al., (1994) Mol. Cell. Biol., 14: 2604-2615). These extracts are then incubated with the synthesized oligonucleotides radiolabeled at their 5′ ends. DNA-protein complexes are separated from unbound DNA by electrophoresis through native polyacrylamide gels and visualized by autoradiography. Binding reactions are performed in the presence of non-radioactive competitor oligonucleotides that bind known transcription factors. In this way we determine the sequence specificity of the binding activities detected and the likelihood they represent factors already cloned. The identity of such factors is confirmed by antibody Supershift analyses performed as we have previously described (Shelley et al., (1992) Leukocyte adhesion 337-351; Bottinger et al., (1994) Mol. Cell. Biol., 14: 2604-2615). DNA-protein complexes formed in nuclear extracts prepared from normal ovarian cells are compared to those formed in extracts of neoplastic ovarian cells to identify differences. Variations in the relative electrophoretic migration of complexes indicate qualitative differences while variations in their relative intensity, as assessed by densitometry, indicate quantitative differences. Once the CD43 gene is found to be mutated and/or abnormally methylated in ovarian cancers, the DNA-protein complexes which form on oligonucleotides containing these mutations and modifications are compared to those that form on oligonucleotides which do not.

UV Crosslinking: EMSA analyses can detect quantitative and gross qualitative differences in nuclear proteins, however, subtle qualitative differences are likely missed. We detect any such differences by determining the molecular weights of the nuclear proteins detected by EMSA (Farokhzad et al., (1996) I. Immunol., 157: 5597-5605; Pongubala et al., (1992) Mol. Cell. Biol., 12: 368-378). This we achieve using the technique of UV crosslinking as we have previously described (Farokhzad et al., (1996) I. Immunol., 157: 5597-5605). Single or double stranded oligonucleotides are synthesized with 5′-bromodeoxyuracil, radiolabeled and binding reactions performed as described for EMSA. After electrophoresis, protein-DNA complexes are irradiated in situ with UV light and visualized by autoradiography. Complexes excised from the gel are electroeluted, precipitated and subjected to SDS-PAGE in parallel with molecular weight markers. Autoradiography is performed again and the sizes of the DNA-binding proteins calculated by subtracting the weight of the oligonucleotide probes. The molecular weights of nuclear proteins expressed in normal and malignant ovarian cells is then compared for differences.

Cloning and Sequencing: The combination of transfection, EMSA, and UV crosslinking analyses described above characterize the cis-acting elements and trans-acting factors responsible for abnormal CD43 gene expression in ovarian cancer. These analyses also determine which trans-acting factors exhibit abnormalities. Such abnormalities are identified by cloning and sequencing.

Factors already known to bind the CD43 gene, such as Sp1, hnRNP-K, Purα; and MeCP2, identified by EMSA will be cloned by reverse transcriptase (RT) PCR using RNA isolated from ovarian cancer cell lines. RT-PCR is also sensitive enough to clone these factors from CD43-positive tumor cells isolated by LCM. The sequences of clones derived from malignant cells are compared to the published wild-type sequences for the identification of mutations.

There are a number of methods to clone unknown transcription factors (Singh, et al., (1988) Cell, 52: 415423; Wang et al, (1993) Nature (London), 364: 121-126; Hoey et al., (1993) Cell, 72: 247-260). However, the most reliable involves classic protein purification and limited sequencing followed by oligonucleotide screening (Hoey et al., (1993) Cell, 72: 247-260). Putative transcription factors are purified from malignant and normal ovarian cell cultures using several chromatographic steps that include ion exchange chromatography, fractionation on heparin agarose and ssDNA-sepharose. Finally, affinity chromatography is performed on a sequence-specific DNA resin consisting of 5′ biotinylated binding-site multimers coupled to streptavidin agarose beads. Purification is followed throughout by EMSA and SDS-PAGE. If multiple bands are seen, each is electroeluted from the gel, renatured (Hager et al., (1980) Anal. Biochem., 109: 76-86) and the gel retardation pattern, produced by each band alone or in combination, determined. Competitions using wild-type and mutant oligonucleotides are used to determine specificity in each case. The appropriate band is then cleaved with trypsin and the amino acid sequences of the resulting peptides determined as we have previously described using a LCQ Decca XP Plus electrospray mass spectroscope (ThermoFinnigan Corp., San Jose, Calif.) set at MS/MS tandem mode (Da Silva et al., (2002) Blood, 100: 3536-3544). Degenerate oligonucleotides is then synthesized based on peptide sequences and used to screen appropriate cDNA libraries. Full-length cDNA inserts are cloned into plasmid vectors which direct in vitro transcription and translation. The sizes of the translated products are then estimated by SDS-PAGE and compared to those of the endogenous proteins as determined by UV crosslinking. The ability of the translated products to form complexes when mixed with wild-type or mutant binding-site oligonucleotides is assessed using EMSA. The nucleotide sequences of clones derived from normal and malignant ovarian cells is compared to identify mutations. Next, factors containing any such mutations are transfected into cultures of normal ovarian cells to determine whether they are capable of activating the CD43 gene.

1. A method for characterizing an ovarian cell, the method comprising:
determining the presence or absence of a CD43 molecule in an ovarian cell of a subject to characterize the ovarian cell.

2. The method of claim 1, wherein the method is diagnostic and wherein the presence of a CD43 molecule in the ovarian cell indicates that the subject has an ovarian tumor.

3. The method of claim 1, wherein the method is prognostic.

4-7. (canceled)

8. The method of claim 1, wherein determining the presence or absence of a CD43 molecule is done in the presence of a CD43 binding molecule.

9-14. (canceled)

15. A method of treating a subject having or at risk of having a tumor, comprising:
administering to a subject in need of such treatment one or more CD43 inhibitors in an effective amount to treat the tumor.

16. The method of claim 15, wherein the CD43 inhibitor is a CD43 nucleic acid binding molecule.

17. The method of claim 16, wherein the CD43 nucleic acid binding molecule is an hnRNP-K molecule.

18-19. (canceled)

20. The method of claim 15, wherein the CD43 inhibitor is transcription factor.

21-39. (canceled)

40. The method of claim 15, wherein the tumor is a solid tumor selected from the group consisting of: biliary tract cancer, brain cancer (including glioblastomas and medulloblastomas), breast cancer, cervical cancer, choriocarcinoma, colon cancer, endometrial cancer, esophageal cancer, gastric cancer, intraepithelial neoplasms, including Bowen's disease and Paget's disease, liver cancer, lung cancer, lymphomas, including Hodgkin's disease and lymphocytic lymphomas, neuroblastomas, oral cancer, including squamous cell carcinoma, ovarian cancer, including those arising from epithelial cells, stromal cells, germ cells and mesenchymal cells, pancreatic cancer, prostate cancer, rectal cancer, renal cancer including adenocarcinoma and Wilms tumor, sarcomas, including leiomyosarcoma, rhabdomyosarcoma, liposarcoma, fibrosarcoma and osteosarcoma, skin cancer, including melanoma, Kaposi's sarcoma, basocellular cancer and squamous cell cancer, testicular cancer, including germinal tumors (seminomas, and non-seminomas such as teratomas and choriocarcinomas), stromal tumors and germ cell tumors, and thyroid cancer, including thyroid adenocarcinoma and medullary carcinoma.

41. The method of claim 15, wherein the tumor is a non-solid tumor selected from the group of hematological neoplasms including: acute or chronic lymphocytic and myelogenous leukemia, multiple myeloma, AIDS associated leukemias and adult T-cell lymphoma/leukemia.

42. The method of claim 15, wherein the tumor is ovarian cancer.

43. The method of claim 15, further comprising administering one or more anti-tumor therapy.

44-61. (canceled)

62. A method for assessing the regression or progression of an ovarian tumor in a subject with comprising the steps of:
measuring in a first ovarian cell obtained from the subject, the presence of a CD43 molecule,
measuring in a second ovarian cell obtained from the subject, the presence of a CD43 molecule,
comparing the presence of the CD43 molecule in the first ovarian cell and the second ovarian cell,
wherein a decrease in the presence of the CD43 molecule in the second ovarian cell compared to the first ovarian cell indicates regression of the ovarian tumor, and
wherein an increase in the presence of the CD43 molecule in the second ovarian cell compared to the first ovarian cell indicates progression of the ovarian tumor.

63-66. (canceled)

67. The method of claim 62, wherein measuring the presence of a CD43 molecule is done in the presence of a CD43 binding molecule.

68-71. (canceled)

72. A kit for diagnosing an ovarian tumor, comprising:
one or more CD43 binding molecules,
one or more control agents, and
instructions for the use of the CD43 binding molecules, and the control agents in the diagnosis of an ovarian tumor.

73-81. (canceled)

82. A method of reducing the number of white blood cells in a subject comprising administering to a subject in need of a reduction in the number of white blood cells, a CD43 inhibitor in an effective amount to reduce the number of white blood cells in said subject.

83. The method of claim 82, wherein the CD43 inhibitor is a hnRNP-K molecule.

84-96. (canceled)

97. The method of claim 82, further comprising administering a second therapy to reduce the number of white blood cells.

98. A method of inhibiting a CD promoter comprising contacting the CD promoter with a CD43 inhibitor in an amount effective to inhibit the CD promoter.

99. The method of claim 98, wherein the CD43 inhibitor is a hnRNP-K molecule.

100-114. (canceled)
